(19) United States
(12) Patent Application Publication
(10) Pub. No.: US 2023/0293966 A1
(43) Pub. Date: Sep. 21, 2023

(54) ORAL DEVICE AND METHODS TO PROTECT THE MOUTH OF A USER

(71) Applicant: NeuroVice, L.L.C., Wake Forest, NC (US)

(72) Inventors: Ashlyn SANDERS, Wake Forest, NC (US); Amanda Hutchenson SCHAFFERS, New Hill, NC (US); David ORENSTEIN, Fuquay-Varina, NC (US); Jay Colton ZIGNEGO, Bahama, NC (US); Jared Andrew DEMORAT, Mountain View, CA (US); Darci Taylor MORELLO, La Jolla, CA (US)

(73) Assignee: NeuroVice, L.L.C., Wake Forest, NC (US)

(21) Appl. No.: 18/298,871

(22) Filed: Apr. 11, 2023

Related U.S. Application Data
(63) Continuation of application No. PCT/US2021/054584, filed on Oct. 12, 2021.
(60) Provisional application No. 63/144,447, filed on Feb. 1, 2021.
(60) Provisional application No. 63/090,255, filed on Oct. 11, 2020.

(51) Int. Cl. A63B 71/08

(52) U.S. Cl. CPC A63B 71/085; A63B 2071/086

(57) ABSTRACT

In some embodiments, an oral device can include a curved frame, a first bite block, and a second bite block. The first bite block can be coupled to a first end of the curved frame and the second bite block can be coupled to a second end of the curved frame. A first wing coupled to the first bite block and a second wing coupled to the second bite block can be configured to corral a tongue of a user such that the tongue of the user is restrained from contacting a tooth engaging surface of the left bite block or the right bite block when the oral device is disposed in a mouth of the user. The oral device can be symmetrical about a plane including a portion of the frame, a portion of the first bite block, and a portion of the second bite block.

### CROSS-REFERENCE TO RELATED APPLICATIONS

This application is a continuation of International Application No. PCT/US2021/054584, filed Oct. 12, 2021, entitled “Oral Device and Methods to Protect the Mouth of a User,” which claims priority to and the benefit of U.S. Provisional Application No. 63/090,255, filed Oct. 11, 2020, entitled “Oral Device and Methods to Protect the Mouth of a User,” and U.S. Provisional Application No. 63/144,447, filed Feb. 1, 2021, entitled “Oral Device and Methods to Protect the Mouth of a User,” the entire contents of each of which are hereby expressly incorporated by reference for all purposes.

### BACKGROUND

Embodiments described herein relate to devices and methods for preventing trauma and injury to the mouth (e.g., tongue, lips, teeth and/or cheeks) of a user experiencing a seizure or other neurologic event characterized by violent contractions, jerking, and/or uncontrolled movements, while minimizing excessive salivation by the user, allowing fluid to drain (e.g., naturally) from the mouth of the user, permitting suctioning of the mouth of the user, and/or permitting fluid (e.g., medication (also referred to as medicament, medicine, pharmaceutical drug, medicinal drug, and/or drug)) delivery to the mouth of the user, the use of which introduces no new risks and poses no hazard to the user.

Seizures can be caused by disorders affecting the brain, including, but not limited to, epilepsy, traumatic brain injury (TBI), brain tumor (e.g., a cancerous brain tumor), stroke, Alzheimer’s disease, eclampsia, and multiple sclerosis. Epilepsy is the most common seizure disorder. Epileptic seizures result from sudden, uncontrolled electrical disturbances in the brain which can cause changes in behavior, body movements, feelings (e.g., sensory perception), and consciousness. Seizures are highly unpredictable and can be variable in symptom presentation, however sufferers can often notice subtle physical, psychological, behavioral or sensory changes, such as an aura, that alert the user to an impending seizure. An electrical disturbance causing a seizure can be limited to one area in the brain or can encompass the entire brain. Seizures are disabling and significantly impact quality of life for sufferers and their caregivers and loved ones. Duration of seizures can range from seconds to several minutes. During a seizure, muscles of the body can alternate between a relaxed state and a rigid state.

Jerking caused by seizures or tremors, for example, can lead to severe tooth, buccal (cheek), lip, and/or tongue injuries. For example, injury to the oral cavity may include painful and irreversible lacerations, soft tissue damage (e.g., tears), loss of teeth, and/or drawing of blood as a consequence of the abnormal, uncontrolled body movements that accompany neurological movement disorders. The uncontrolled body movements can include lip smacking, chewing, automatisms, jerking, and other motor components. Open wounds and lacerations also are associated with infection risks and recurrent injury resulting from use of the tongue and mouth, which can lead to prolonged healing times and potentially a need for costly additional care and treatment.

Disorders causing uncontrolled movements of the mouth can arise due to disturbances to the central nervous system (e.g., Parkinson’s disease) or to brain function (e.g., epilepsy) (as described above) and may arise due to genetics or following injury, autoimmune diseases, infections, and/or as a pharmacological effect. Further, excessive salivation, pooling of saliva, and drooling (i.e., sialorrhea) usually manifests during these episodes of uncontrolled movement. In particular, tonic-clonic seizures (the most common form of seizures) can cause hypersalivation and can induce jerking movements that pose a high risk of trauma and injury to the oral cavity, including the tongue and cheeks. These seizures tighten muscles in the jaw and face, causing the person to bite down incessantly in the form of a chewing motion, regardless of tongue or cheek position.

Although the current practice is to avoid placing objects in the mouth of a patient seizing, sufferers, bystanders, and caregivers have resorted to using hazardous objects such as spoons, towels, pencils, and socks to provide a barrier between the tongue and other areas of the mouth to prevent tooth injury. Additionally, sufferers, bystanders, and caregivers have resorted to repurposing dental guards, mouthguards, and bite blocks to prevent injury to their tongue and cheeks. These devices are medically and financially unsuitable to prevent injury to patients with seizure and/or movement disorders, as the repurposed devices are designed with the primary functionality of addressing corrective or aesthetic dental needs. As such, the application of such devices only applies when the body and oral muscles are relaxed, rather than a situation in which the body is moving violently, contracting, and/or jerking uncontrollably. Additionally, such devices often require an expensive fitting to configure the device. Furthermore, dental appliances made for dental applications can be bulky and uncomfortable, and are commonly made of materials or polymers that are not conducive to the compressive forces between the jaws during a seizure and thus these repurposed devices become easily deformed and torn, increasing the risk of a choking or swallowing hazard. These devices also fail to prevent oral fluids (i.e., saliva and/or vomit) from collecting in the mouth, causing a choking and breathing hazard. Furthermore, such devices can cause choking, cause or fail to prevent cracking of teeth, and can block the user’s airway. Currently, seizure safety guidelines discourage placing any objects in the mouth of a person experiencing a seizure.

As one of the most sensitive organs of the body, trauma or injury to the oral cavity significantly impacts quality of life, causing severe pain and discomfort to patients, impeding their ability to perform basic tasks such as talking and eating. Oral injuries can also create additional anxiety and feelings of embarrassment for patients with epilepsy and other seizure/movement disorders. Thus, a significant unmet need exists for a medical device that safely and effectively prevents oral injury and/or allows oral fluids to be suctioned or drained during seizures, tremors, and other movement disorders.

### SUMMARY

Apparatus and methods are described herein for preventing trauma and injury to the oral cavity of a user experiencing a seizure or other neurologic event characterized by excessive and/or uncontrolled movements, and preventing the collection of oral fluids (e.g., saliva and/or vomit) in the mouth of the user, while introducing no hazards to the user. In some embodiments, an oral device can include a curved frame, a first bite block, and a second bite block. The first bite block can be coupled to a first end of the curved frame and the second bite block can be coupled to a second end of the curved frame. A first wing coupled to the first bite block and a second wing coupled to the second bite block can be configured to corral a tongue of a user such that the tongue of the user is restrained from contacting a tooth engaging surface of the left bite block or the right bite block when the oral device is disposed in a mouth of the user. The oral device can be symmetrical about a plane including a portion of the frame, a portion of the first bite block, and a portion of the second bite block.

### BRIEF DESCRIPTION OF THE DRAWINGS

FIG. 1 is a schematic illustration of an oral device, according to an embodiment.

FIG. 2 is a schematic illustration of the oral device of FIG. 1 as part of a kit, according to an embodiment.

FIG. 3 is a flow chart illustrating a method, according to an embodiment.

FIG. 4 is a schematic illustration of the teeth of a typical mouth, according to an embodiment.

FIGS. 5-11 are various views of an oral device, according to an embodiment.

FIGS. 12-13 are top views of the oral device of FIG. 5 in a first configuration and a second configuration, respectively.

FIGS. 14-20 are various views of an oral device, according to an embodiment.

FIGS. 21-27 are various views of an oral device, according to an embodiment.

FIGS. 28-34 are various views of an oral device, according to an embodiment.

FIGS. 35-41 are various views of an oral device, according to an embodiment.

FIGS. 42-48 are various views of an oral device, according to an embodiment.

FIGS. 49 and 50 are front perspective views of a kit, according to an embodiment.

FIG. 51 is a front perspective view of an oral device, according to an embodiment.

FIG. 52 is a front perspective view of an oral device, according to an embodiment.

FIG. 53 is a front perspective view of an oral device, according to an embodiment.

FIG. 54 is a side view of an oral device, according to an embodiment.

FIG. 55 is a front view of an oral device, according to an embodiment.

FIGS. 56 to 59 are side and front views, respectively, of an oral device, according to an embodiment.

FIGS. 60 and 61 are front perspective views of an oral device, according to an embodiment.

FIG. 62 is a front perspective view of an oral device, according to an embodiment.

FIGS. 63 and 64 are a front open view and a front closed perspective view, respectively, of an oral device, according to an embodiment.

FIGS. 65 and 66 are front perspective views of an oral device, according to an embodiment.

FIGS. 67 and 68 are front perspective and side views, respectively, of an oral device, according to an embodiment.

FIG. 69 is a front perspective view of an oral device, according to an embodiment.

FIG. 70 is a front perspective view of an oral device, according to an embodiment.

FIG. 71 is a front perspective view of an oral device, according to an embodiment.

FIGS. 72 and 73 are front perspective views of an oral device, according to an embodiment.

FIG. 74 is a front perspective view of an oral device, according to an embodiment.

FIGS. 75 and 76 are a front perspective view of an oral device in a first configuration and a second configuration, respectively, according to an embodiment.

FIGS. 77A and 77B are front views of an oral device in a first configuration and a second configuration, respectively, according to an embodiment.

FIGS. 78-80 are various views of an oral device, according to an embodiment.

FIGS. 81-82 are front perspective and front cross-sectional views, respectively, of an oral device, according to an embodiment.

FIG. 83 is a posterior schematic view of an oral device, according to an embodiment.

FIG. 84 is a front perspective view of an oral device, according to an embodiment.

FIG. 85 is a front view of an oral device, according to an embodiment.

FIG. 86 is a perspective view of an oral device, according to an embodiment.

FIG. 87 is a front perspective view of an oral device, according to an embodiment.

FIGS. 88A and 88B are various views of an oral device, according to an embodiment.

FIGS. 89A and 89B are various views of an oral device, according to an embodiment.

FIGS. 90A and 90B are various views of an oral device, according to an embodiment.

FIG. 91 is a perspective view of an oral device, according to an embodiment.

FIGS. 92A and 92B are various views of an oral device, according to an embodiment.

FIG. 93A is a front perspective view of an oral device, according to an embodiment.

FIG. 93B is a rear perspective view of an oral device, according to an embodiment.

FIG. 94 is a front perspective view of an oral device, according to an embodiment.

FIG. 95A is a front perspective view of an oral device disposed within a storage container, according to an embodiment.

FIG. 95B is a front perspective view of an oral device, according to an embodiment.

FIGS. 96A and 96B are various views of an oral device, according to an embodiment.

FIGS. 97-100B are various views of an oral device, according to an embodiment.

FIGS. 101-102B are front perspective, top, and bottom views, respectively, of an oral device, according to an embodiment.

FIGS. 103A-104B are various views of the oral device of FIGS. 101-102B.

FIG. 105 is a flow chart illustrating a method of using an oral device, according to an embodiment.

### DETAILED DESCRIPTION

An oral device and method of use are disclosed that provide both a structural and functional response for the problems of tongue biting, cheek biting, and/or hazardous teeth clamping (during seizures and other conditions in which the body experiences spasmodic attacks, jerking, teeth clenching, uncontrollable movements that increase the likelihood of biting the tongue), and collection of saliva in the mouth due to hypersalivation. The device is safe and effective for outpatient and clinical use, is easily insertable into the mouth by a user or assistant, has a simple, intuitive structure, and is cost-effective. In some embodiments, the device is single-use and disposable. In some embodiments, the device can be custom fitted to a user’s mouth and reusable. In some embodiments, the device can function as a clinical and/or household aid in preventing trauma and/or injury to the tongue and in preventing the collection of the saliva due to hypersalivation during seizures.

In some embodiments, an oral device includes a curved frame, a first bite block, and a second bite block. The first bite block can be coupled to a first end of the curved frame and the second bite block can be coupled to a second end of the curved frame. A first wing coupled to the first bite block and a second wing coupled to the second bite block can be configured to corral a tongue of a user such that the tongue of the user is restrained from contacting a tooth engaging surface of the left bite block or the right bite block when the oral device is disposed in a mouth of the user. The oral device is symmetrical about a plane including a portion of the frame, a portion of the first bite block, and a portion of the second bite block. The oral device includes a handle coupled to and extending distally from a distal surface of the curved frame a sufficient distance such that, when the oral device is disposed on the user’s mouth, a distal portion of the handle extends from the user’s mouth and can be grasped by the user.

In some embodiments, an oral device includes a curved frame, a first bite block, and a second bite block. The first bite block can be coupled to a first end of the curved frame and the second bite block can be coupled to a second end of the curved frame. The oral device also includes a handle coupled to and extending distally from the curved frame. The handle has a first handle portion and a second handle portion, the first handle portion configured to be urged toward the second handle portion to transition the curved frame from a first configuration in which the first end and the second end of the curved frame are a first distance apart to a second configuration in which the first end and the second end of the curved frame are a second distance apart greater than the first distance.

In some embodiments, a method includes identifying a subject experiencing a seizure or at least one warning sign of a seizure and inserting an oral device into engagement with a tongue and teeth of the subject. The oral device includes a curved frame having a first end and a second end, a first bite block coupled to the first end of the curved frame, and a second bite block coupled to the second end of the curved frame. The oral device also includes a first wing coupled to the first bite block and a second wing coupled to the second bite block. The first wing and the second wing are configured to corral a tongue of the subject such that the tongue of the subject is restrained from contacting a tooth engaging surface of the left bite block or the right bite block when the oral device is disposed in a mouth of the subject, and a handle extending distally from the curved frame a sufficient distance such that a distal portion of the handle can be grasped between an index finger and a thumb when the oral device is disposed in the subject’s mouth.

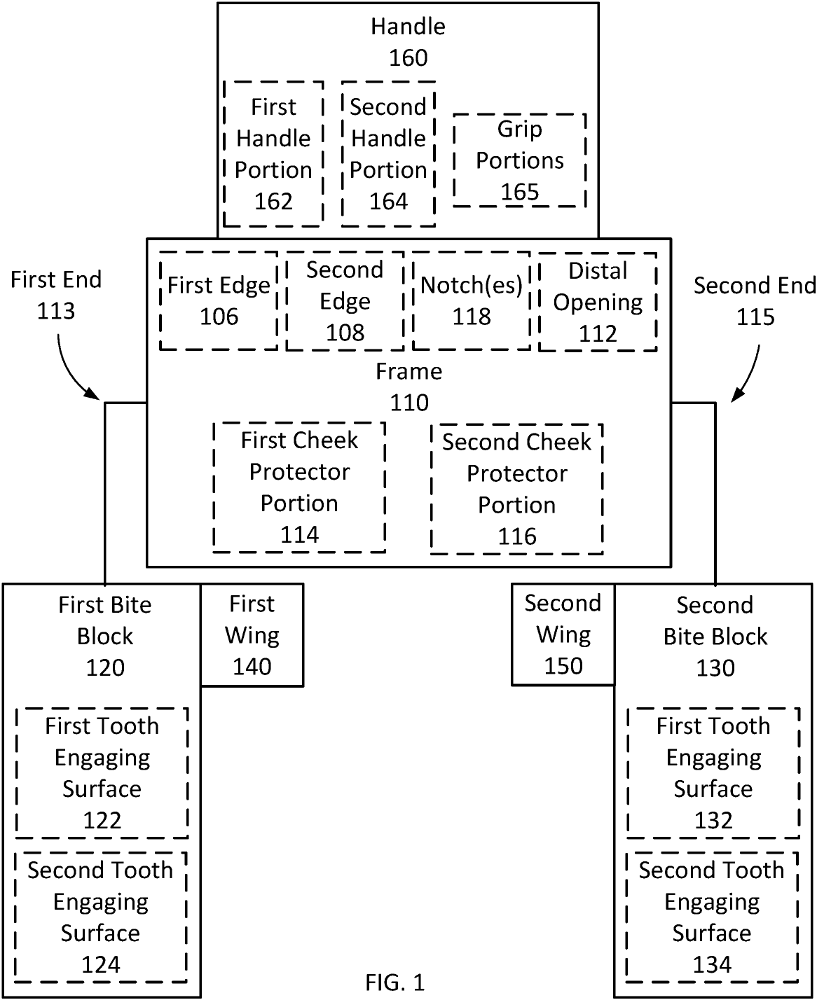

FIG. 1 is a schematic illustration of an oral device 100. The oral device 100 includes a curved frame 110, a first bite block 120, and a second bite block 130. The curved frame 110 has a first end 113 and a second end 115. The first bite block 120 is coupled to the first end 113 of the curved frame 110 and the second bite block 130 is coupled to the second end 115 of the curved frame 110. In some embodiments, the first bite block 120 can be coupled to the second bite block 130 only via the curved frame 110. The oral device 100 includes a first wing 140 coupled to the first bite block 120 and a second wing 150 coupled to the second bite block 130. The oral device 100 can also include a handle 160. In some embodiments, the frame 110 can optionally define a distal opening 112.

The handle 160 can be gripped by the administrator of the oral device 100 (e.g., a user or a caregiver) for safe insertion, positioning, and removal of the oral device 100 within the mouth of a user (also referred to herein as a “subject”) without having to dispose fingers of the administrator in a bite path of the user. The caregiver can include, for example, a doctor, nurse, emergency medical technician (EMT), home health aide, lay family member, lay friend, or lay community member (e.g., a “Good Samaritan” stranger). The handle 160 can extend distally from the curved frame 110. For example, the handle 160 can extend a sufficient distance from the curved frame 110 such that the handle 160 protrudes from the user’s mouth when the oral device 100 is disposed within the mouth of the user. The handle 160 is configured to be gripped (e.g., by a user or caregiver) during insertion and positioning of the oral device 100 within the mouth of the user and removal of the oral device 100 from the mouth of the user. In some embodiments, the oral device 100 (e.g., the handle 160) does not include any portion and/or is not coupled to any component configured to contact a surface of the user’s face (e.g., a non-lip surface or portion) when the oral device 100 is disposed in the user’s mouth.

The handle 160 can define a recess or opening aligned with a distal opening 112 of the frame 110. For example, in some embodiments, the handle 160 can project distally from the frame 110 and partially or fully surround the distal opening 112. The handle 160 can define an opening or recess that is U-shaped or semi-circular in an upper and/or lower surface of the handle 160. In some embodiments, the handle 160 can define a gap or space between portions of the handle such that an upper and lower surface of the distal opening 112 is defined by only the frame 110 and not by the handle 160.

The handle 160 can optionally include a first handle portion 162 and a second handle portion 164. In some embodiments, the first handle portion 162 and the second handle portion 164 can each be formed as an extension, prong, or tab extending distally away from the frame 110. In some embodiments, a gap or space is defined between the first handle portion 162 and the second handle portion 162. In some embodiments, the first handle portion 162 and the second handle portion 164 can be included in a continuous extension portion (e.g., a substantially circular extension portion) extending distally from the frame 110. In some embodiments, the first handle portion 162 and the second handle portion 164 include portions extending partially around a distal-facing surface of the frame 110 (e.g., laterally away from the distal opening 112). In some embodiments, the handle 160 is not detachable from the curved frame 110 (e.g., the handle 160 is monolithically formed with the curved frame 110 or non-releasably engaged). In some embodiments, the handle 160 is removable from the curved frame 110 (e.g., via a tearaway portion including perforations). In some embodiments, the handle 160 can be configured to maintain a separation between the user’s lips, thus allowing for oral fluids to be drawn or delivered and for breathing to be unobstructed through the distal opening 112. In some embodiments, the handle 160 can include a tubular central portion aligned with the distal opening 112 and a tab portion extending from each side of the tubular central portion. The tubular central portion can define a U-shaped recess in a top and bottom surface of the tubular central portion such that the minimum distance from the proximal or inside surface of the frame 110 to the distal edge of the handle 160 defining an opening aligned with the distal opening 112 is minimized.

The handle 160 can extend away from the distal surface of the frame 110 a sufficient distance such that a distal portion of the handle 160 can be grasped by fingers of the user or caregiver (e.g., an index finger and a thumb) when the oral device 100 is disposed in the user’s mouth and the lips of the user are contacting or near proximal portions of the handle 160. In some embodiments, the handle 160 can protrude from the user’s mouth no more than a distance sufficient to grasp the handle 160 between fingers (e.g., two fingertips) (e.g., with the upper and lower lips of the user closed against or contacting a proximal portion of the handle 160), which may prevent the user or caregiver from inserting the oral device 100 too far into the user’s mouth such that a distal portion of the frame 110 is disposed proximal of the user’s front teeth, and can also reduce the risk of inadvertent, external forces being applied to the handle 160 (e.g., during seizure) that might dislodge the oral device 100 relative to the user’s mouth. In some embodiments, the handle 160 can extend away from the distal surface of the frame 110 a distance of about 15.7 mm. In some embodiments, the handle 160 can extend away from the distal surface of the frame 110 a distance of between about 1 cm and about 2 cm, a distance of between 17 mm and about 20 mm, a distance of about 20 mm and 25 mm, or any other suitable distance.

In some embodiments, the frame 110 can include a first cheek protector portion 114 and a second cheek protector portion 116. The first cheek protector portion 114 can extend from a distal center portion of the frame 110 to a proximal edge of the first bite block 120 and the second cheek protector 116 can extend from a distal center portion of the frame 110 to a proximal edge of the second bite block 130. In some embodiments, the first cheek protector portion 114 and the second cheek protector portion 116 can each include a portion that tapers proximally to facilitate insertion of the oral device 100 into the user’s mouth and/or to correspond to the shape of the user’s mouth cavity. In some embodiments, the frame 110 can define one or more notches 118. For example, a first notch 118 can be defined along a first edge 106 of a center portion of the frame 110 and a second notch 118 can be defined along a second edge 108 of a center portion of the frame 110 opposite the first edge 106 (e.g., each aligned vertically with a central axis of the distal opening 112). In some embodiments, the notches 118 can be configured to receive the upper and lower labial frenulum of the user. In some embodiments, the notches 118 can aid in encouraging the frame 110 to bend about an axis through the notches 118. In use, the frame 110 can be disposed between an outer surface of the subject’s teeth and an inner surface of the user’s lips and/or cheeks to prevent contact between the user’s teeth and the inner surface of the user’s lips and/or cheeks.

The distal opening 112 can be configured such that, when the oral device 100 is disposed within the user’s mouth, the distal opening 112 aligns with a gap defined between the upper and lower teeth of the user due to the upper and lower teeth being urged apart by the oral device 100. In some embodiments, the distal opening 112 is defined between the first handle portion 162 and the second handle portion 164. In some embodiments, the distal opening 112 can be round (e.g., ovular or circular), rectangular, or any other suitable shape. In some embodiments, the distal opening 112 can be sufficiently large to provide access to the mouth of a user through the distal opening 112 when the oral device 100 is disposed in the mouth of the user. For example, the distal opening 112 can be configured (e.g., shaped and sized) to allow for the natural flow of air, saliva, and vomit out of the mouth during the seizure. In some embodiments, the distal opening 112 can be sufficiently large and the handle 160 can define a sufficiently large gap, recess, or space in communication with (e.g., aligned with or adjacent to) the distal opening 112 such that the inside of the user’s mouth can be sufficiently visualized by a caregiver to determine if the mouth or an airway of the user is obstructed and/or if suction is required. The distal opening 112 can be configured to receive a portion of a catheter such that fluid can be transferred relative to the mouth of the user via the catheter (e.g., saliva can be suctioned from the mouth and/or medication can be delivered to the mouth). In some embodiments, the distal opening 112 can be at least 6 mm wide at its smallest dimension. In some embodiments, the distal opening 112 can be sufficiently large to accommodate an 18 French catheter. In some embodiments, the distal opening 112 can be sufficiently large to accommodate a catheter of any suitable size, such as a catheter that is equal to, greater than, or less than 22 French, 20 French, 19 French, 17 French, 16 French, 15 French, 14 French, 13 French.

In some embodiments, a combination of the shape and size of the distal opening 112, the shape and size of the handle 160, and the diameter of the catheter allows for a particular lateral and vertical range of motion of the catheter relative to the distal opening to access various portions of the mouth. For example, in some embodiments, for example due to the recess, opening, space, or gap defined by the handle 160 above and below the distal opening 112, a catheter (e.g., an 18 French catheter) can have a vertical range of motion (e.g., in a vertical plane including only the frame 110 and optionally the handle 160) of about 60°, about 65°, about 70°, about 75°, about 80°, about 85°, about 90°, about 95°, or about 100°. In some embodiments, a catheter (e.g., an 18 French catheter) can have a horizontal or lateral range of motion (e.g., in a horizontal plane perpendicular to the vertical plane and including the handle 160, the frame 110, the first bite block 120, the second bite block 130, the first wing 140, and the second wing 150) of about 60°, about 65°, about 70°, about 75°, about 80°, about 85°, about 90°, about 95°, or about 100°. In some embodiments, the device 100 allows for a larger vertical range of motion than a horizontal range of motion within the distal opening 112 (e.g., due to the portions of the handle 160 projecting from the curved frame 110 on either side of the distal opening 112). In some embodiments, a combination of the shape and size of the distal opening 112, the shape and size of the handle 160, and the diameter of the catheter allows for sufficient visualization by a caregiver beyond the handle 160 and through the distal opening 112 for the caregiver to be able to determine if the mouth or an airway of the user is obstructed and/or if suction is required when the device 100 is disposed in the user’s mouth.

The first bite block 120 includes a first tooth engaging surface 122 and a second tooth engaging surface 124 opposite the first tooth engaging surface 122 and the second bite block 130 includes a first tooth engaging surface 132 and a second tooth engaging surface 134 opposite the first tooth engaging surface 132. In some embodiments, the first tooth engaging surface 122 of the first bite block 120 can be parallel to the second tooth engaging surface 124 of the first bite block 120 (e.g., the first tooth engaging surface 122 can be disposed in a plane lying parallel to a plane in which the second tooth engaging surface 124 is disposed). In some embodiments, the first tooth engaging surface 132 of the second bite block 130 can be parallel to the second tooth engaging surface 134 of the second bite block 130 (e.g., the first tooth engaging surface 132 can be disposed in a plane lying parallel to a plane in which the second tooth engaging surface 134 is disposed). In some embodiments, the first tooth engaging surface 122 can be disposed at a non-zero angle relative to the second tooth engaging surface 124 and the first tooth engaging surface 132 can be disposed at a non-zero angle relative to the second tooth engaging surface 134 such that a distal end of each of the first bite block 120 and the second bite block 130 has a greater height than a proximal end of each of the first bite block 120 and the second bite block 130.

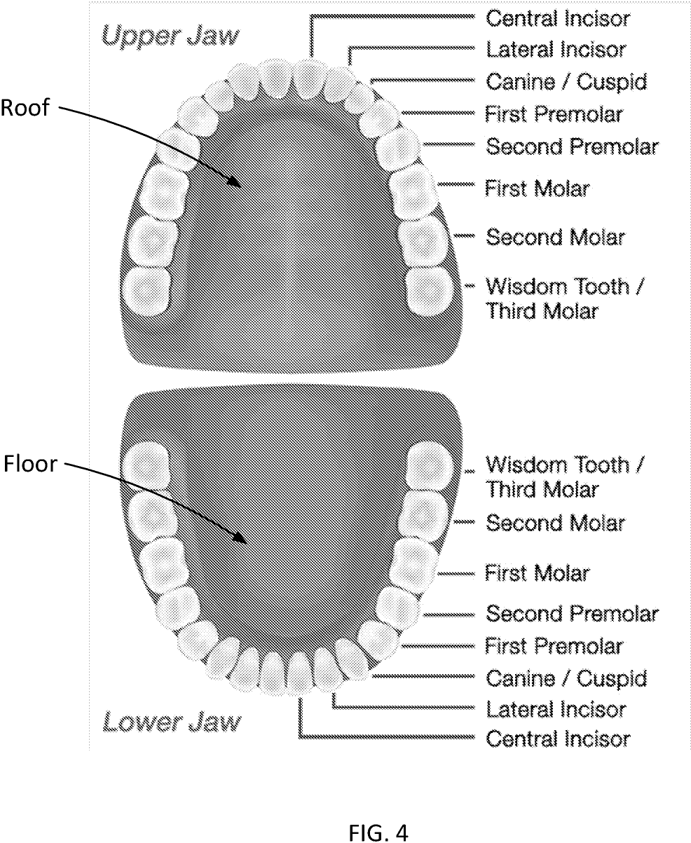

The first bite block 120 and the second bite block 130 can be configured (e.g. shaped and sized) to be contacted by any suitable tooth or range of teeth. For example, in some embodiments, the first bite block 120 and the second bite block 130 can be shaped and sized such that the first bite block 120 and the second bite block 130 can be disposed between and contacted by the left and right upper and lower molars (e.g., the first and second premolars and first and second molars as shown in FIG. 4) and canines of the user, respectively. Said another way, the first bite block 120 and the second bite block 130 can be disposed between and in contact with teeth on the upper and lower left and right side of a user’s mouth, respectively, leaving the user’s incisors non-contacted. In some embodiments, the first bite block 120 and the second bite block 130 can be shaped to be contacted by the user’s range of teeth from the upper and lower canine to the upper and lower third molar (if the user has a lower third molar) on each side of the user’s mouth. In some embodiments, the first bite block 120 and the second bite block 130 can be shaped and sized to be disposed in contact with only the upper and lower molars or the upper and lower canines. In some embodiments, the first bite block 120 and the second bite block 130 can be shaped and sized to be disposed in contact with only the upper and lower molars and none, one, or both of the upper and lower premolars on each side. The first bite block 120 and the second bite block 130 can have any suitable cross-sectional shape (e.g., rectangular, square, circular, triangular, ovular).

In some embodiments, the first bite block 120 and the second bite block 130 can be formed of a material with a high compressive strength to resist compression by the teeth during a biting or chewing movement. In some embodiments, the first bite block 120 and the second bite block 130 can be formed of an elastomeric mesh material. In some embodiments, the first bite block 120, the second bite block 130, and optionally the remaining portions of the device 100 can be formed, for example, of a thermoplastic elastomer such as Versalloy™. In some embodiments, the first bite block 120 and the second bite block 130 can have a higher elasticity than the frame 110. In some embodiments, the first bite block 120 and the second bite block 130 can each define one or more perforations or apertures extending from the respective first tooth engaging surface (i.e., surfaces 122 or 132) to the respective second tooth engaging surface (i.e., surfaces 124 or 134). In some embodiments, the apertures or perforations can be configured to function as breathing vents such that air can flow through the apertures or perforations. Fluid can flow through the apertures to reduce the buildup of saliva in a user’s mouth which may occur as a result of excessive salivation (e.g., during seizures). In some embodiments, rather than defining apertures or perforations extending entirely through the first bite block 120 and the second bite block 130, each of the first bite block 120 and the second bite block 130 can define an array of cavities extending partially through the first bite block 120 and the second bite block 130, respectively. Each of the cavities can be configured to receive and contain liquid (e.g., saliva and/or medicament). One or more cavities defined in a first side of a bite block (e.g., surfaces 122 or 132) can be spaced from a cavity defined in a second side of the bite block (e.g., surfaces 124 or 134) (e.g., the opposing cavities can be coaxial and distinct from each other).

The first wing 140 and the second wing 150 can be coupled to and/or extend from an inside edge of the first bite block 120 and the second bite block 130, respectively. In some embodiments, the first wing 140 and/or the second wing 150 can be referred to separately or collectively as a cradle, coraller, cradle portion, or coraller portion. The first wing 140 and the second wing 150 are configured to corral a tongue of a user such that the tongue of the user is restrained from contacting a tooth engaging surface of the first bite block 120 (i.e., surfaces 122 or 124) or the second bite block 130 (i.e., surfaces 132 or 134) when the oral device 100 is disposed in a mouth of the user and the user moves his or her jaw (and, thus, teeth) relative to (e.g., separates the teeth from) the first bite block 120 and/or the second bite block 130 (e.g., during a biting, chewing, talking and/or clamping motion). Thus, in some embodiments, even if the user opens his or her mouth to a maximum natural extent, the tongue of the user can be prevented from entering a space between the teeth of the user and the first bite block 120 and/or the second bite block 130 by the first wing 140 and/or the second wing 150. The first wing 140 can be curved away from the first bite block 120 (e.g., toward the second wing 150) and the second wing 150 can be curved away from the second bite block 130 (e.g., toward the first wing 140). Thus, the first wing 140 and the second wing 150 can each have a convex inner surface defining a curved receiving area to receive a respective side of a user’s tongue and can each form a boundary of movement of the tongue with respect to the first bite block 120 and the second bite block 130 such that the tongue cannot move laterally beyond the first wing 140 and the second wing 150 to contact a surface of the first bite block 120 or the second bite block 130. In some embodiments, the first wing 140 and the second wing 150 can apply pressure to opposing sides of the tongue of the user to immobilize or reduce a range of motion (e.g., a lateral range of motion) of the tongue or at least a back portion of the tongue. In some embodiments, the user’s tongue can contact the roof and/or floor of the user’s mouth when braced between the first wing 140 and the second wing 150. In some embodiments, the first wing 140 can extend from a distal edge to a proximal edge of the first bite block 120 (e.g., such that the first wing 140 does not extend proximally or distally of the first bite block 120) and the second wing 150 can extend from a distal edge to a proximal edge of the second bite block 130 (e.g., such that the second wing 150 does not extend proximally or distally of the second bite block 130).

In some embodiments, each of the first wing 140 and the second wing 150 can have a distal edge extending vertically upward and downward in a plane including a distal edge of the first bite block 120 and the second bite block 130, respectively. In some embodiments, each of the first wing 140 and the second wing 150 can have an upper edge, a lower edge, an upper tapering portion, and a lower tapering portion. The upper and lower tapering portions can extend from the upper edge and the lower edge of the first wing 140 and the second wing 150 to a proximal edge of the first bite block 120 and the second bite block 130, respectively.

In some embodiments, each of the first wing 140 and the second wing 150 can have a sufficient height (i.e., a sufficient vertical extent relative to the first bite block 120 and the second bite block 130), such that, when the device 100 is disposed in the user’s mouth, the first wing 140 and the second wing 150 each contact the roof (also referred to as the palate) and/or the floor of the user’s mouth. For example, an upper edge of each of the first wing 140 and the second wing 150 contact the roof of the user’s mouth and a lower edge of each of the first wing 140 and the second wing 150 contact the floor of the user’s mouth. In some embodiments, the first wing 140 and/or the second wing 150 can have a height that is about equal to or greater than two times the height of the first bite block 120 and the second bite block 130, respectively. In some embodiments, the first wing 140 and/or the second wing 150 can have a sufficient height such that the first wing 140 and/or the second wing 150 extend at least to a midpoint of the upper teeth and/or a midpoint of the lower teeth. In some embodiments, the first wing 140 and/or the second wing 150 can have a sufficient height such that the first wing 140 and/or the second wing 150 extend at least to the upper gumline and/or the lower gumline of the user. In some embodiments, the first wing 140 and/or the second wing 150 can have a height that is about equal to or greater than the height (e.g., the maximum or average height) of the curved frame 110.

In some embodiments, two portions of the handle 160 (e.g., the first handle portion 162 and the second handle portion 164) are configured to be urged toward each other to cause the frame 110 to contract from a first configuration in which the first end 113 and the second end 115 of the frame 110 are a first distance apart to a second configuration in which the first end 113 and the second end 115 of the frame 110 are a second distance apart smaller than the first distance. In some embodiments, the two portions of the handle 160 can be rotated toward each other (e.g., by pressing proximal portions of the handle portions toward one another) such that a portion of the curved frame and/or a portion of the handle 160 between the first and second portion of the handle 160 (e.g., a tubular central portion disposed between, for example, two tab-shaped handle portions) functions as a hinge. In some embodiments, the two portions of the handle 160 can be translated toward each other such that a portion of the curved frame and/or a portion of the handle 160 between the first and second portion of the handle 160 (e.g., a tubular central portion disposed between, for example, two tab-shaped handle portions) is compressed (e.g., laterally) and the first end 113 and the second end 115 of the frame 110 are moved closer together. In some embodiments, the oral device 100 can include or be formed of a shape memory material. In some embodiments, the oral device 100 and/or the frame 110 can be biased toward the first configuration. Thus, in some embodiments, the oral device 100 can be transitioned from the first configuration to the second configuration for insertion into the user’s mouth, and then allowed to transition from the second configuration to the first configuration when within the user’s mouth for engagement with the user’s teeth.

In some embodiments, two portions of the handle 160 (e.g., the first handle portion 162 and the second handle portion 164) are configured to be urged toward each other to cause the frame 110 to expand from a first configuration in which the first end 113 and the second end 115 of the frame 110 are a first distance apart to a second configuration in which the first end 113 and the second end 115 of the frame 110 are a second distance apart greater than the first distance. In some embodiments, the two portions of the handle 160 can be rotated toward each other (e.g., by pressing distal portions of the handle portions toward one another) such that a portion of the curved frame and/or a portion of the handle 160 between the first and second portion of the handle 160 (e.g., a tubular central portion disposed between, for example, two tab-shaped handle portions) functions as a hinge. In some embodiments, the oral device 100 can include or be formed of a shape memory material. In some embodiments, the oral device 100 and/or the frame 110 can be biased toward the first configuration. Thus, in some embodiments, the oral device 100 can be transitioned from the first configuration to the second configuration for insertion into the user’s mouth, and then allowed to transition from the second configuration to the first configuration when within the user’s mouth for engagement with the user’s teeth.

In some embodiments, the distal portions of the first handle portion 162 and the second handle portion 164 can be urged toward each other to cause the ends of the frame 110 to rotate away from each other and the frame 110 to expand from the first configuration to the second configuration in which the distance between the ends of the frame 110 is larger than in the first configuration. In some embodiments, the two portions of the handle 160 can be rotated toward each other (e.g., by pressing distal portions of the handle portions toward one another) such that a portion of the curved frame and/or a portion of the handle 160 between the first and second portion of the handle 160 (e.g., a tubular central portion disposed between, for example, two tab-shaped handle portions) functions as a hinge. The proximal portions of the first handle portion 162 and the second handle portion 164 can be urged toward each other to urge the ends of the frame 110 toward each other and the frame 110 to contract from the first configuration to a third configuration in which the distance between the ends of the frame 110 is smaller than in the first configuration. In some embodiments, the two portions of the handle 160 can be rotated toward each other (e.g., by pressing proximal portions of the handle portions toward one another) such that a portion of the curved frame and/or a portion of the handle 160 between the first and second portion of the handle 160 (e.g., a tubular central portion disposed between, for example, two tab-shaped handle portions) functions as a hinge. The frame 110 can be transitioned to the second configuration or the third configuration for insertion into the user’s mouth, and then transitioned to one of the other of the first, second, or third configurations within the user’s mouth. In some embodiments, a force can be applied to an inner surface of the first handle portion 162 and/or the second handle portion 164 away from the other of the first handle portion 162 or the second handle portion 164 to cause the frame 110 to contract to the third configuration. In some embodiments, the two portions of the handle 160 can be translated toward each other such that a portion of the curved frame and/or a portion of the handle 160 between the first and second portion of the handle 160 (e.g., a tubular central portion disposed between, for example, two tab-shaped handle portions) is compressed (e.g., laterally) and the first end 113 and the second end 115 of the frame 110 are moved closer together (e.g., into a fourth configuration which may be similar to or different from the third configuration).

In some embodiments, the handle 160 can include a plurality of grip portions (e.g., raised portions or ridges) disposed along the handle 160 (e.g., along the first handle portion 162 and the second handle portion 164) to assist the user in gripping the handle 160. In some embodiments, the grip portions can be formed as lettering or words (e.g., instructions) identifying the device 100 or instructions for use of the device 100 (e.g., “DO NOT REUSE”). In embodiments in which the frame 110 includes notches 118 in a center portion of the frame 110, the notches 118 can assist the frame 110 in transitioning between the first configuration to the second configuration due to the frame 110 contracting and/or expanding (e.g., hinging) about an axis extending through the first notch 118 and the second notch 118. In some embodiments, the oral device 100 can be transitioned between a first and second configuration before, during, and/or after placement of the oral device 100 in the user’s mouth.

In some embodiments, the first wing 140, the first bite block 120, and a first end 113 portion of the frame 110 can collectively form a first channel and a second channel configured to receive a first set of upper teeth and a first set of lower teeth of the user. The second wing 150, the second bite block 130, and a second end 115 portion of the frame 110 can collectively form a third channel and a fourth channel configured to receive a second set of upper teeth and a second set of lower teeth of the user. As a result of the first set of upper teeth, the first set of lower teeth, the second set of upper teeth, and the second set of lower teeth being disposed in the first channel, the second channel, the third channel, and the fourth channel, respectively, the device 100 can be secured and maintained in the mouth of the user and/or can have limited movement relative to the teeth of the user (e.g., due to the user applying a compressive force to the first bite block 120 and the second bite block 130 with the user’s teeth).

In some embodiments, the oral device 100 can be symmetrical about a plane (e.g., a horizontal plane) including a portion of the frame 110, a portion of the first bite block 120, and a portion of the second bite block 130. In some embodiments, the oral device 100 can be symmetrical about a plane (e.g., a horizontal plane) including a portion of the frame 110, a portion of the first bite block 120, a portion of the second bite block 130, a portion of the first wing 140, a portion of the second wing 150, and/or a portion of the handle 160. Thus, the oral device 100 can be inserted into the mouth of the user in one of two orientations and function the same way in either orientation. For example, the oral device 100 can be inserted such that the first bite block 120 is configured to be engaged by the left teeth of the user and the second bite block 130 is configured to be engaged by the right teeth of the user, and the oral device can be inserted such that the first bite block 120 is configured to be engaged by the right teeth of the user and the second bite block 130 is configured to be engaged by the left teeth of the user, eliminating the need for a user or a caregiver to determine the proper orientation of the oral device 100 before insertion into the user’s mouth. In some embodiments, the oral device 100 may not be symmetrical about a horizontal plane and instead may have a top surface intended for engagement with a user’s upper teeth and a bottom surface intended for engagement with a user’s bottom teeth.

In use, the oral device 100 can prevent the damage from teeth to teeth, teeth to cheek, teeth to lip, and/or teeth to tongue impact that can occur during biting or chewing (e.g., during a seizure). For example, during a seizure, damage to the teeth and/or lateral lacerations and/or other injuries to the cheek or tongue can be caused by the molars and/or canines. Due to the surface area ratio, the lower molars (i.e., molars and premolars) and canines can experience the most impact during the chewing motion. Additionally, lacerations and/or other injuries to the tip of the tongue can be caused by the incisors. The oral device 100 can prevent damage and injury to the teeth and tongue via the dissipation and mitigation of the force of impact between, for example, the top and bottom molars and canines and between the teeth and the tongue. In use, the oral device 100 can allow the user to swallow (e.g., swallow saliva) when the oral device 100 is disposed within the user’s mouth. The user can inhale and exhale when the oral device is disposed within the user’s mouth (e.g., via the distal opening 112). The user can bite the oral device 100 and relax the user’s jaw repeatedly without the oral device 100 dislodging or becoming loose within the user’s mouth.

The oral device 100 can be configured to not generate particulate that poses a choking hazard during typical biting patterns. For example, when placed under a force of 148 N or 774 N (e.g., by a simulated jaw or a user’s jaw) cycled at a rate of 1.5 Hz for 150 s (such as during a tonic-clonic seizure), the oral device 100 does not generate particulate that poses a choking hazard. As another example, when placed under a force of 148 N or 774 N (e.g., by a simulated jaw or a user’s jaw) applied constantly for 150 s (such as during a tonic seizure), the oral device 100 does not generate particulate that poses a choking hazard. The oral device 100 also does not generate particulate when soaked in saliva or preconditioned (e.g., for two hours by being submerged in distilled water at 37 +/- 2° C.) prior to the application of the bite force. The oral device 100 can be formed without latex and such that the oral device 100 is compliant with ISO 10993-1 so as not to illicit a biological reaction from the user.

In some embodiments, the first bite block 120 and the second bite block 130 can each be sufficiently thick (e.g., have sufficient height) to absorb the force of the top teeth impacting the lower teeth such that the top teeth and the lower teeth are not damaged. In some embodiments, the first bite block 120 and the second bite block 130 can be shaped and sized such that the tongue (e.g., the tip of the tongue) is prevented from being disposed in the space between and simultaneously contacted by the upper teeth and the lower teeth with enough force to injure or damage the tongue. For example, the first bite block 120 can be sufficiently thick (e.g., have sufficient height) between the first tooth engaging surface 122 of the first bite block 120 and the second tooth engaging surface 124 of the first bite block 120 and the second bite block 130 can be sufficiently thick (e.g., have sufficient height) between the first tooth engaging surface 132 of the second bite block 130 and the second tooth engaging surface 134 of the second bite block 130 such that a user cannot bite the tongue of the user when the oral device 100 is disposed in the mouth of the user due to the upper teeth and the lower teeth being prevented from simultaneously contacting the tongue or simultaneously contacting the tongue with enough force to injure the tongue. In some embodiments, the first bite block 120 is sufficiently thick between the first tooth engaging surface 122 of the first bite block 120 and the second tooth engaging surface 124 of the first bite block 120 and the second bite block 130 is sufficiently thick between the first tooth engaging surface 132 of the second bite block 130 and the second tooth engaging surface 134 of the second bite block 130 such that, when the oral device 100 is disposed in the mouth of the user, the user is prevented from simultaneously contacting and/or applying a force (e.g., a biting force) to the tongue of the user with an upper front tooth and a lower front tooth sufficient to damage the tongue.

In some embodiments, the dimensions of the oral device 100 can be selected such that the oral device 100 can conform to the mouth size of an adult (e.g., a typical adult mouth size). In some embodiments, the dimensions of the oral device 100 can be selected such that the oral device 100 can conform to the mouth size of a child (e.g., a typical child mouth size). In some embodiments, the dimensions of the oral device 100 can be selected such that the oral device 100 can conform to the mouth size of an adolescent (e.g., a typical adolescent mouth size). In some embodiments, the dimensions of the oral device 100 can be configured as needed based on the mouth size of a user (i.e., custom fitted to the user). In some embodiments, the overall length of the oral device can be at least about 31.75 mm (1.25″) in at least two particular planes to prevent choking. In some embodiments, the overall length of the oral device 100 can be greater than or equal to about 30.85 mm and less than or equal to about 47.14 mm from the inside surface of the frame 110 to the proximal edge of the device 100. In some embodiments, the oral device 100 can include flavoring materials. In some embodiments, the oral device 100 can be available in a variety of colors or combinations of colors. In some embodiments, the oral device 100 can include embossed or engraved designs. In some embodiments, the oral device 100 can be formed of an absorbent material (e.g., an absorbent polymer such as a food grade super absorbent polymer) such that saliva can be absorbed by the oral device 100 during use.

In some embodiments, the oral device 100 can be configured to be disposed within the mouth of a user for a single use period. For example, the oral device 100 can be configured to be disposed within the mouth of the user during a single event of use, such as during an episode of movement disorder (e.g., seizures) or during a period of sleep. The oral device 100 can be configured to be discarded (i.e., thrown away) after a single-use period. For example, the oral device 100 can be constructed from a material that is susceptible to bacterial infection, and thus should be discarded after the single event of use. In some implementations, the oral device 100 can be configured to transition from an un-activated configuration (e.g., a pre-use configuration) to an activated configuration (e.g., a use configuration) but configured not to transition from the activated configuration to the un-activated configuration after the initial transition to the activated configuration. For example, the oral device 100 can include a seal within the oral device 100 (e.g., between components of the oral device 100) in the activated configuration that must be broken to transition the oral device 100 from the un-activated to the activated configuration such that the oral device is disposed in a suitable shape to be disposed within the mouth of the user. In some embodiments, the shelf life of the oral device 100 can be greater than one year.

The oral device 100 can be worn for any suitable duration of time. In some circumstances, patients who have been diagnosed with a neurological condition that causes seizures can consult with their physician and identify possible triggers or warning sign factors before seizure onset. Such warning signs can include, for example, a rising feeling in the stomach, temporary confusion, an unresponsiveness or staring spell, uncontrollable jerking or twitching movements or stiffness of the arms and legs, loss of consciousness or awareness, cognitive or emotional symptoms (e.g., a sudden intense feeling of joy, fear, anxiety, or déjà vu), a strange feeling like a wave going through the head, headache, trouble sleeping, nausea, unusual smells, tastes, sounds, or sensations, visual disturbances such as colored or flashing lights or hallucinations, a sensation of an arm or leg feeling bigger than its true size, numbness or tingling sensations in certain parts of the body, and/or weakness. Such people, when experiencing a trigger or an aura, can insert the oral device 100 into his/her mouth before a seizure begins. For users who do not experience warning signs prior to seizure onset, or users with conditions resulting in continuous uncontrollable movements that may result in tongue injury, the device can be worn at all times and removed only for limited periods, such as, for example, for eating. In some embodiments, the oral device 100 can be worn while sleeping (e.g., to prevent injury that may occur during nocturnal seizures). Due to the structure of the oral device 100, the user can position the oral device 100 quickly and easily without requiring a fitting by a specialist or teeth impressions taken in advance. Said another way, the oral device 100 can be an off-the-shelf device that can be configured to engage with a number of potential user’s mouths. In some embodiments, the oral device 100 can be retailed over the counter. In some embodiments, the oral device 100 can be prescribed by and/or administered by a physician or other healthcare provider (e.g., a doctor or nurse). In some embodiments, the oral device 100 can be available to and administered by first responders, paramedics, and EMT staff. For example, the oral device 100 can be included in first aid kits and/or emergency response kits. The oral device 100 can be an emergency medical intervention for such personnel to administer to seizure patients during a response to a seizure-related and/or head injury-related emergency call or incident.

In some embodiments, the handle 160, the frame 110, and/or a portion of the handle 160 and/or frame 110 defining the distal opening 112 can be shaped (e.g., sufficiently wide) to prevent the lips of the user from contacting each other when the oral device 100 is disposed in the user’s mouth, allowing for drainage of saliva from the user’s mouth and/or safe insertion of a catheter or medication into the user’s mouth (e.g., even if the user’s jaw is clenched). The oral device 100 can be available to healthcare providers, such as physicians or nurses, in hospitals, and/or in clinics for use in patients during an electroencephalogram (EEG) test of the patient (e.g., the oral device 100 can be placed in the patient’s mouth prior to the start of the test) which may induce a seizure, during a sleep study of the patient to evaluate a sleep-related seizure disorder (e.g., the oral device 100 can be placed in the patient’s mouth prior to the start of the sleep study), and/or who are designated for “seizure precautions” (e.g., in a seizure toolkit in the emergency department or hospital admission) for use in a patient who is experiencing an ongoing seizure and/or while a patient who had a seizure is being evaluated further. In some embodiments, a person other than the user can insert the oral device 100 into the user’s mouth before or during a seizure. For example, a caregiver or a person made aware of the user’s need for the oral device 100 based on the user’s symptoms and/or a signifier of the user (e.g., an instruction bracelet, necklace, or wallet card describing the user’s medical condition and/or need for use of the oral device 100) may insert the oral device into the user’s mouth. In some embodiments, the oral device 100 can be administered to stroke patients or patients requiring oral motor therapy or speech therapy to supplant tongue rehabilitation and improve tongue strength lost due to tongue paresthesia or numbness.

In some embodiments, one or more of the wings 140, 150 and/or bite blocks 120, 130 can include an electromechanical component (not shown). The electromechanical component can cause, for example, vibration of the wings and/or bite blocks. The electromechanical component can also provide mobility and support for the wings 140, 150 (and the user’s tongue when engaged with the tongue wings 140, 150). Thus, users who have tongue paresthesia or tongue weakness, such as epileptic or stroke patients, can use the oral device 100 to rehabilitate tongue strength.

In some embodiments, the electromechanical component can include a motor controlled by a printed circuit board (PCB) and powered by a power source. A vibrating element, such as, for example, a small weight, can be attached to the motor. The electromechanical component can also include a housing in which the motor, power source, vibrating element, and/or PCB can be disposed. The power source can be, for example, a small, rechargeable battery. The battery can be selected to optimize space efficiency and power. The PCB can control the motor to provide various patterns and multiple speeds of vibration. In some embodiments, a single button can be used to perform multiple functions (e.g., turn the motor on/off and select one of the various patterns and multiple speeds of vibration). In some embodiments, the PCB can include two or more controls to induce specific changes to the length and resistance of the wires and/or circuit of the PCB to adjust the pattern and speed of vibration of the vibrating element. The housing can be relatively thin and can be formed of, for example, plastic or another rigid material.

In some embodiments, the electromechanical component, or a portion of the electromechanical component, can be encapsulated by the material (e.g., polymer) of one of the bite blocks 120, 130 and/or the wings 140, 150. Although only one electromechanical component has been described, in some embodiments each of the first bite block 120 and first wing 140 and the second bite block 130 and second wing 150 can be associated with a separate electromechanical component.

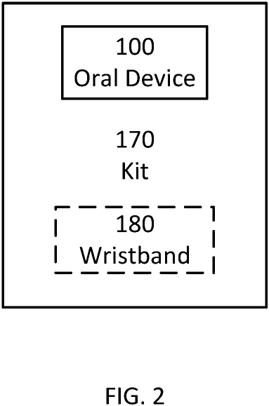

As shown in FIG. 2, in some embodiments the oral device 100 can be disposed inside a kit 170. The kit 170 can include a case or container that can be used to protect the oral device 100 when the oral device 100 is not being used. In some embodiments, the case or container can be shaped and sized to fit within a bag, purse, or pocket. In some embodiments, the case or container can be worn around a neck of a user for quick accessibility. The kit 170 can optionally include a wristband 180. The wristband 180 can include a label that informs a reader that the user/wearer has the oral device 100 and/or that instructs the reader to insert the oral device 100 into the mouth of the user in the event of a particular event occurring (e.g., a seizure). Thus, other people and/or healthcare professionals can be made aware that the user has the oral device 100 in the event that the user feels that a seizure will occur or the user starts to have a seizure. In some embodiments, the kit 170 can include more than one oral device 100. Each oral device 100 in the kit 170 can be individually packaged to maintain the sterility of each oral device 100 until needed for use. In some embodiments, the case or container can be attached to the wristband 180. In some embodiments, the case or container can include an antenna and/or a transmitter or transceiver such that a location of the case or container can be monitored via GPS monitoring.

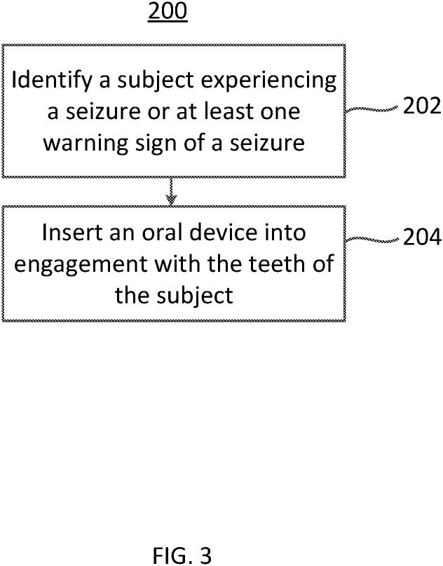

FIG. 3 is a flow chart illustrating a method 200. As shown in FIG. 3, the method 200 includes identifying 202 a subject (also referred to as a user) experiencing a seizure or at least one warning sign of a seizure. In some embodiments, the subject experiencing a seizure or at least one warning sign of a seizure can be identified based on the subject exhibiting (in a manner noticeable by a person other than the subject, such as a caregiver), or based on the subject himself or herself recognizing, one or more symptoms or signs of a seizure, such as any of the symptoms or signs described herein. In some embodiments, the subject experiencing a seizure or at least one warning sign of a seizure can be identified based on the subject communicating (e.g., verbally or by gesturing toward an identifier on, for example, a bracelet) that he or she is experiencing a seizure or at least one warning sign of a seizure, or based on the subject communicating (e.g., verbally or by gesturing toward an identifier on, for example, a bracelet) that he or she is experiencing one or more symptoms or signs of a seizure, such as any of the symptoms or signs described herein.

In some embodiments, the identifying can be performed by the subject. In some embodiments, the identifying can be performed by a person other than the subject (e.g., a caregiver such as a family member, a friend, a medical practitioner, or a stranger). An oral device can be inserted 204 into engagement with the teeth of the subject. In some embodiments, the oral device can be the same or similar in structure and/or function to any of the oral devices described herein, such as the oral device 100, the oral device 300, and/or the oral device 4500. For example, an oral device, such as any of the oral devices described herein, can be inserted into a subject’s mouth such that bite blocks of the oral device are disposed between a subject’s upper and lower teeth, and such that a curved frame of the oral device 100 is disposed between an outer surface of the subject’s teeth and an inner surface of the subject’s lips and/or cheeks. In some embodiments, the oral device can be inserted such that wings of the oral device are disposed between the sides of the subject’s tongue and the inner surface of the subject’s teeth. In some embodiments, the oral device can be inserted by grasping a handle coupled to and extending distally from a distal surface of the curved frame (e.g., via grasping the handle between an index finger and thumb using one or both hands) and inserting the oral device into the subject’s mouth. The handle can extend from the curved frame a sufficient distance such that, when the oral device is disposed in the subject’s mouth, a distal portion of the handle extends from the subject’s mouth and can be grasped by the subject or a caregiver such that the oral device can be inserted into the user’s mouth without the subject or caregiver’s fingers entering the subject’s mouth or being disposed in a bite path of the subject.

In some embodiments, the handle coupled to and extending distally from the curved frame can have a first handle portion and a second handle portion, and the subject or caregiver can urge the first handle portion toward the second handle portion to transition the curved frame from a first configuration in which the first end and the second end of the curved frame are a first distance apart to a second configuration in which the first end and the second end of the curved frame are a second distance apart greater than the first distance. In some embodiments, a catheter can be inserted through an opening in the curved frame. In some embodiments, fluid can be drawn from the mouth through the catheter. In some embodiments, medicament can be provided to the mouth through the catheter. In some embodiments, an end of the catheter can be manipulated laterally and vertically within the mouth relative to the opening in the curved frame. In some embodiments, the catheter has a larger range of motion vertically than laterally with respect to the opening in the curved frame. In some embodiments, the identifying includes matching an identifier associated with the subject (e.g., on a bracelet, necklace, or wallet card) of the subject to an identifier on the oral device (e.g., disposed on packaging of the oral device or on the oral device itself). In some embodiments, the oral device can be removed from engagement with the tongue and teeth of the subject via grasping the handle between an index finger and thumb and pulling the oral device from the subject’s mouth, and disposing of the oral device after one use of the oral device. In some embodiments, inserting the oral device includes grasping the handle between the index finger and the thumb and disposing the oral device in engagement with the tongue and the teeth of the subject without inserting the index finger or the thumb into the subject’s mouth. In some embodiments, inserting the oral device includes moving a first handle portion relative a second handle portion (as described above with respect to various configurations of the oral device 100) to transition the curved frame from a first configuration in which the first end and the second end of the curved frame are a first distance apart to a second configuration in which the first end and the second end of the curved frame are a second distance apart and inserting the oral device into the subject’s mouth. In some embodiments, the second distance is greater than the first distance. In some embodiments, the first distance is greater than the second distance. In some embodiments, an oral medicament can be administered to the mouth of the subject through an opening defined in the curved frame. The oral medicament can be the same or similar as any of the oral medicaments described herein, such as an oral medicament formed as a tablet, pill, or an oral medicament dispensable in liquid form through the opening.

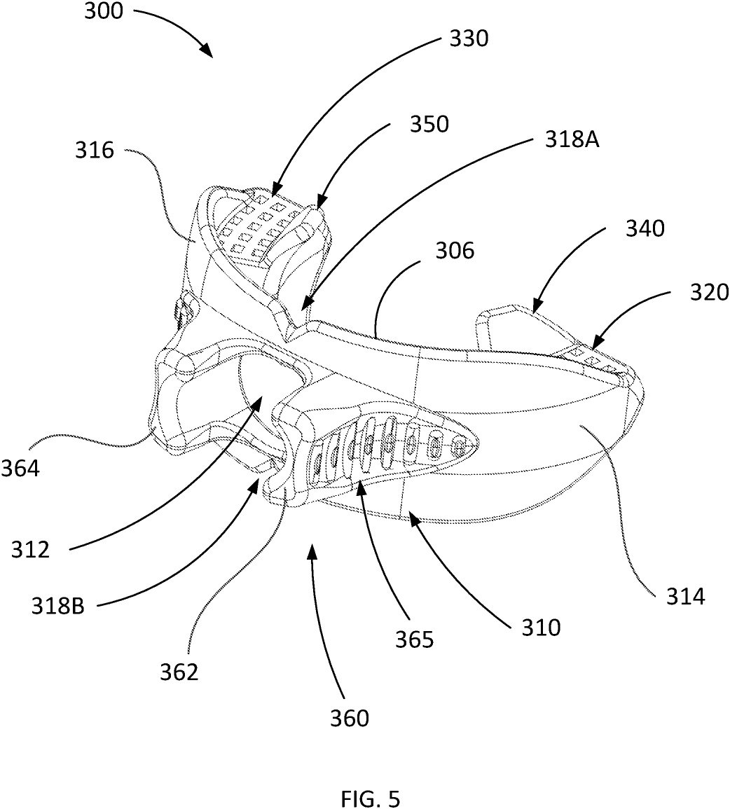

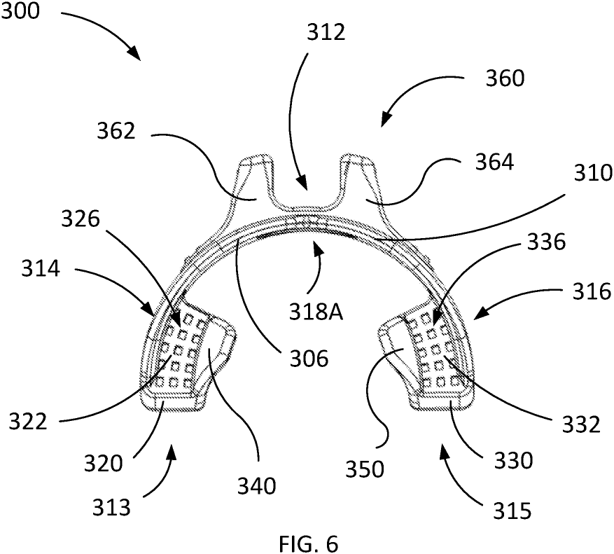

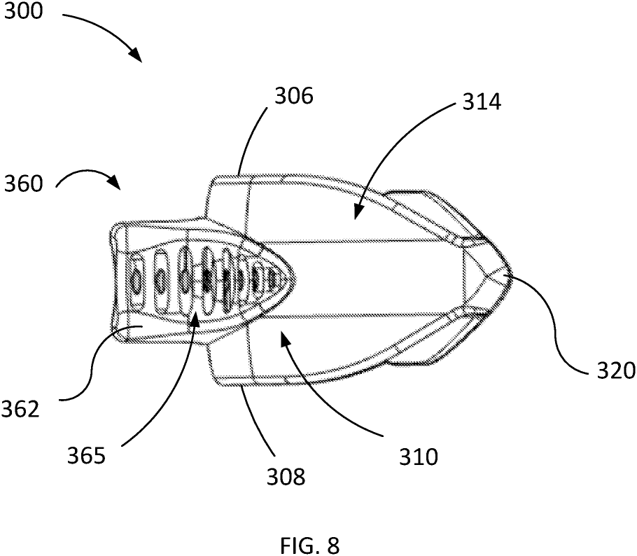

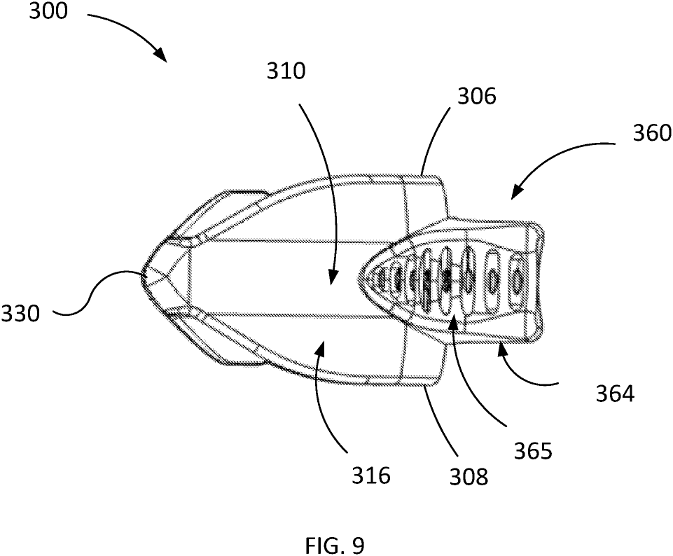

FIGS. 5-11 are front perspective, top, bottom, first side, second side, front, and rear views, respectively, of an oral device 300. The oral device 300 can be the same or similar in structure and/or function to any of the oral devices described herein, such as the oral device 100. For example, the oral device 300 includes a curved frame 310, a first bite block 320, and a second bite block 330 that can be the same or similar in structure and/or function to the curved frame 110, the first bite block 120, and the second bite block 130, respectively, described above with respect to FIG. 1. As shown in FIG. 6, the curved frame 310 has a first end 313 and a second end 315. The curved frame 310 is configured to be disposed between a user’s teeth and lips. The first bite block 320 is coupled to the first end 313 of the curved frame 310 and the second bite block 330 is coupled to the second end 315 of the curved frame 310. The oral device 300 also includes a first wing 340 and a second wing 150 that can be the same or similar in structure and/or function to the first wing 140 and the second wing 150 described above with respect to the oral device 100. For example, the first wing 340 can be coupled to the first bite block 320 and the second wing 350 can be coupled to the second bite block 330. In use, the oral device 300 can allow the user to swallow (e.g., swallow saliva) when the oral device 300 is disposed within the user’s mouth.

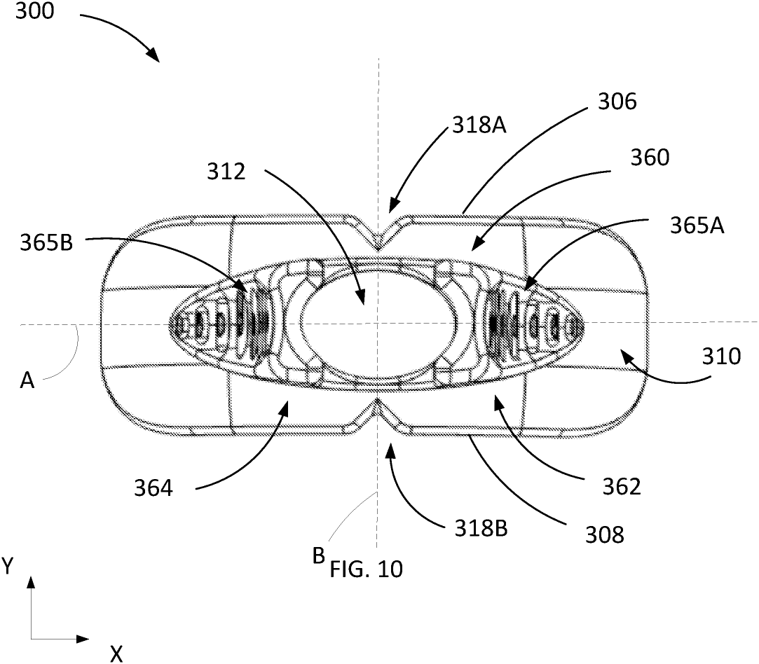

The oral device 300 also includes a handle 360 that may be the same or similar in structure and/or function to the handle 160 described above with respect to the oral device 100. In some embodiments, the frame 310 can optionally define a distal opening 312.The handle 360 can be gripped by the administrator of the oral device 300 (e.g., the user or a caregiver) for safe insertion, positioning, and removal of the oral device 300 without having to dispose fingers of the administrator in a bite path of the user. The handle 360 extends distally from the curved frame 310. The handle 360 can extend a sufficient distance from the curved frame 310 such that the handle 360 protrudes from the user’s mouth when the oral device 300 is disposed within the mouth of the user. The handle 360 is configured to be gripped (e.g., by a user or caregiver) during insertion, removal, and positioning of the oral device 300 within the mouth of the user. The handle 360 includes a first handle portion 362 and a second handle portion 364. As shown in FIG. 6, the first handle portion 362 and the second handle portion 364 each include an extension or prong extending distally away from the frame 310. As shown in FIG. 6, a gap or space is defined between the first handle portion 362 and the second handle portion 362 that aligns with the opening 312 (described below). As shown in FIG. 10, the first handle portion 362 and the second handle portion 364 include a first grip portion 365A and a second grip portion 365B, respectively. The first grip portion 365A extends from the extension portion of the first handle portion 362 partially toward the first end 313 of the curved frame 310 around a distal-facing surface of the frame 310. The second grip portion 365B extends from the extension portion of the second handle portion 364 partially toward the second end 315 of the curved frame 310 around a distal-facing surface of the frame 310. In some embodiments, the handle 360 is not detachable from the curved frame 310 (e.g., the handle 360 is monolithically formed with the curved frame 310 or non-releasably engaged). In some embodiments, the handle 360 is removable from the curved frame 310 (e.g., via a tearaway portion including perforations). The handle 360 can be configured to maintain a separation between the user’s lips, thus allowing for oral fluids to drain, be drawn (e.g., suctioned), and for breathing to be unobstructed through the distal opening 312.

The frame 310 can include a first cheek protector portion 314 and a second cheek protector portion 316. The first cheek protector portion 314 can extend from a distal center portion of the frame 310 to a proximal edge of the first bite block 320 and the second cheek protector 316 can extend from a distal center portion of the frame 310 to a proximal edge of the second bite block 330. The first cheek protector portion 314 and the second cheek protector portion 316 can each include a portion that tapers proximally to facilitate insertion of the oral device 300 into the user’s mouth and/or to correspond to the shape of the user’s mouth cavity. As shown in FIG. 5, the frame 310 can define a first notch 318A defined along a first edge 306 of a center portion of the frame 310 and a second notch 318B along a second edge 308 of a center portion of the frame 310 opposite the first edge 306. The first notch 318A and the second notch 318B can be configured to receive the upper and lower labial frenulum of the user.

As shown in FIG. 5, the distal opening 312 can be configured such that, when the oral device 300 is disposed within the user’s mouth, the distal opening 312 aligns with a gap defined between the upper and lower teeth of the user. The distal opening 312 is defined between the first handle portion 362 and the second handle portion 364. The distal opening 312 can be round (e.g., ovular or circular). The distal opening 312 is sufficiently large to provide access to the mouth of the user through the distal opening 312 when the oral device 300 is disposed in the mouth of the user. For example, the distal opening 112 can be configured to allow for the natural flow of air, saliva, and vomit out of the mouth during the seizure. The distal opening 312 can be configured to receive a portion of a catheter such that fluid can be transferred relative to the mouth of the user via the catheter (e.g., saliva can be suctioned from the mouth and/or medication can be delivered to the mouth).

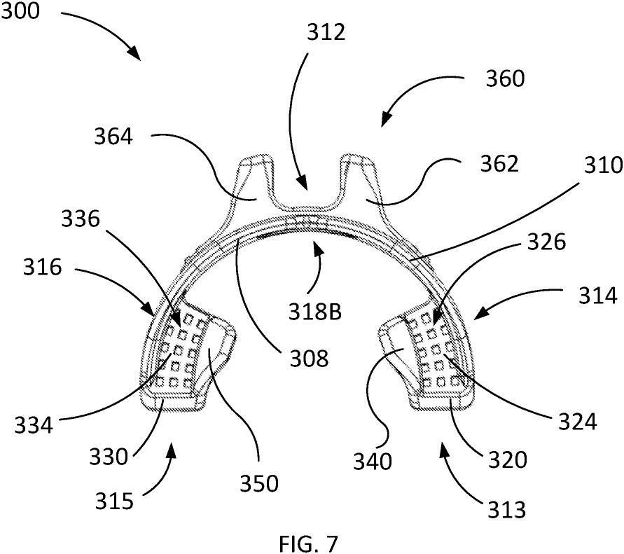

As shown in FIGS. 6-7, the first bite block 320 includes a first tooth engaging surface 322 and a second tooth engaging surface 324 opposite the first tooth engaging surface 322 and the second bite block 330 includes a first tooth engaging surface 332 and a second tooth engaging surface 334 opposite the first tooth engaging surface 332. The first tooth engaging surface 322 of the first bite block 320 is disposed parallel to the second tooth engaging surface 324 of the first bite block 320 (e.g., the first tooth engaging surface 322 can be disposed in a plane lying parallel to a plane in which the second tooth engaging surface 324 is disposed). The first tooth engaging surface 332 of the second bite block 330 can be parallel to the second tooth engaging surface 334 of the second bite block 330 (e.g., the first tooth engaging surface 332 can be disposed in a plane lying parallel to a plane in which the second tooth engaging surface 334 is disposed).

The first bite block 320 and the second bite block 330 can be configured (e.g. shaped and sized) to be contacted by any suitable tooth or range of teeth. For example, in some embodiments, the first bite block 320 and the second bite block 330 can be shaped and sized such that the first bite block 320 and the second bite block 330 can be disposed between and contacted by the left and right upper and lower molars (e.g., the first and second premolars and first and second molars as shown in FIG. 4) and canines of the user, respectively. Said another way, the first bite block 320 and the second bite block 330 can be disposed between and in contact with teeth on the upper and lower left and right side of a user’s mouth, respectively, leaving the user’s incisors non-contacted. In some embodiments, the first bite block 320 and the second bite block 330 can be shaped to be contacted by the user’s range of teeth from the upper and lower canine to the upper and lower third molar (if the user has a lower third molar) on each side of the user’s mouth. In some embodiments, the first bite block 320 and the second bite block 330 can be shaped and sized to be disposed in contact with only the upper and lower molars or the upper and lower canines.

In some embodiments, the first bite block 320 and the second bite block 330 can be formed of a material with a high compressive strength to resist compression by the teeth during a biting or chewing movement. In some embodiments, the first bite block 320 and the second bite block 330 can be formed of an elastomeric mesh material. In some embodiments, the first bite block 320 and the second bite block 330 can have a higher elasticity than the frame 310. In some embodiments, the first bite block 320 and the second bite block 330 can each define one or more perforations or apertures 326, 336 extending from the respective first tooth engaging surfaces 322, 332 to the respective second tooth engaging surfaces 324, 334. The apertures or perforations can be configured to function as breathing vents such that air can flow through the apertures or perforations. Fluid can flow through the apertures 326, 336 to reduce the buildup of saliva in a user’s mouth which may occur as a result of excessive salivation (e.g., during seizures).

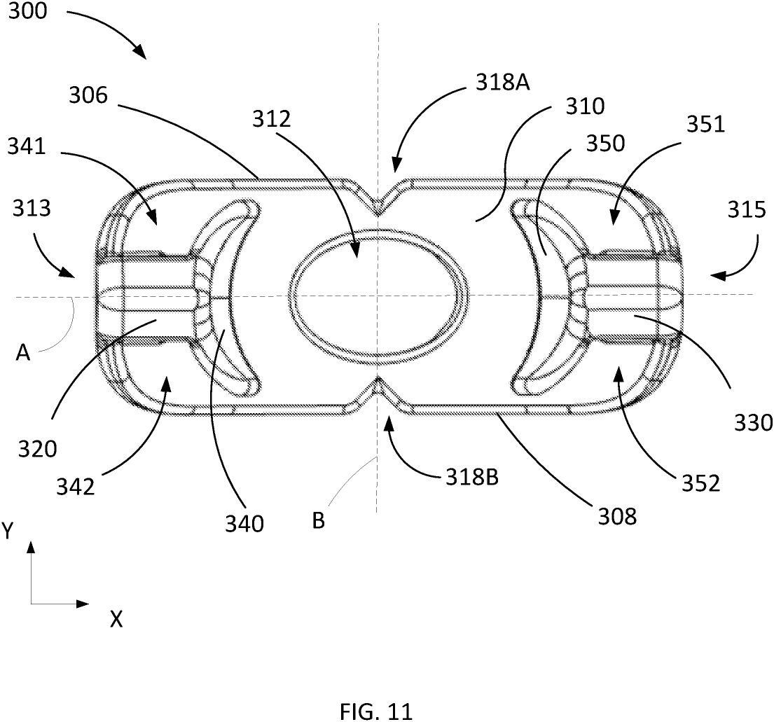

The first wing 340 and the second wing 350 are configured to corral a tongue of a user such that the tongue of the user is restrained from contacting the first tooth engaging surface 322 and the second tooth engaging surface 324 of the first bite block 320 or the first tooth engaging surface 332 and the second tooth engaging surface 334 of the second bite block 330 when the oral device 300 is disposed in a mouth of the user. As shown, for example, in FIG. 11, the first wing 340 is curved away from the first bite block 320 and the second wing 350 can be curved away from the second bite block 330. Thus, the first wing 340 and the second wing 350 can each define a curved receiving area to receive a respective side of a user’s tongue and can each form a boundary of movement of the tongue with respect to the first bite block 320 and the second bite block 330. As shown, for example, in FIG. 6, the first wing 340 can extend from a distal edge to a proximal edge of the first bite block 320 (e.g., such that the first wing 340 does not extend proximally or distally of the first bite block 320) and the second wing 350 can extend from a distal edge to a proximal edge of the second bite block 330 (e.g., such that the second wing 350 does not extend proximally or distally of the second bite block 330).

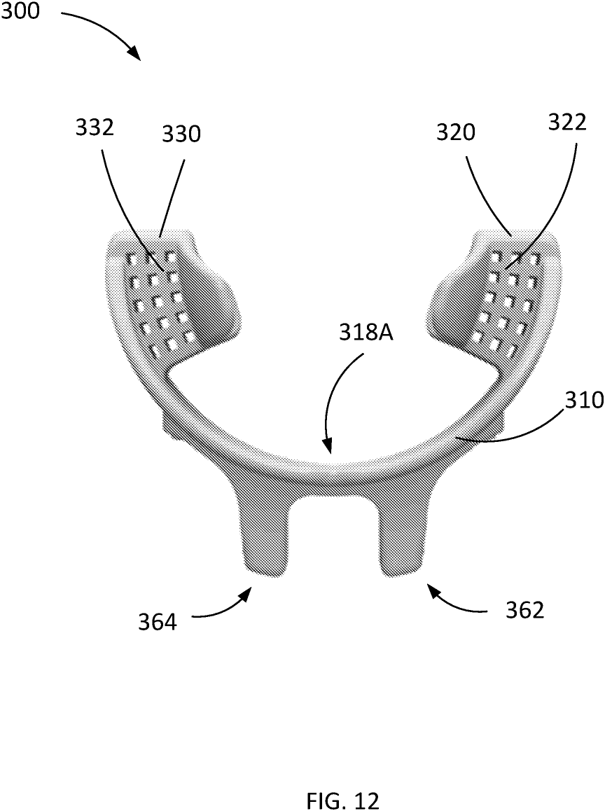

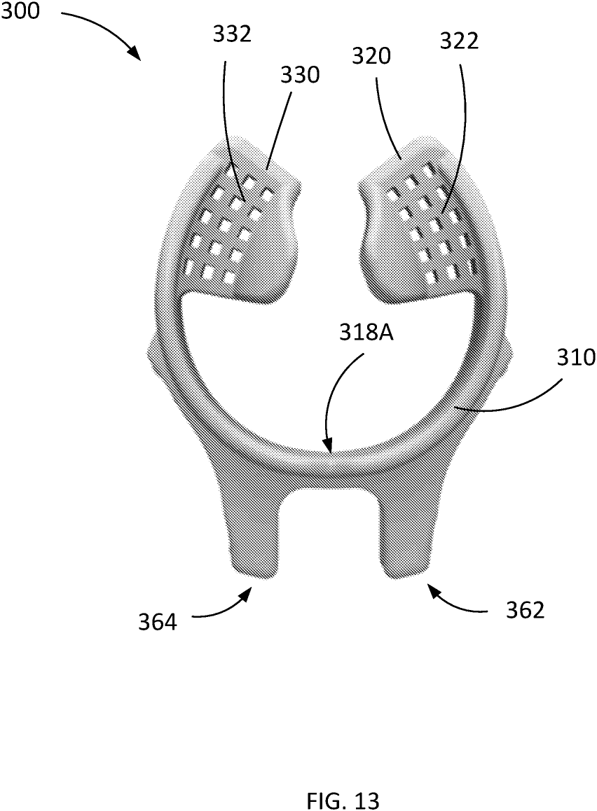

FIGS. 12-13 are top views of an oral device 300 in a first and second configuration, respectively. In some embodiments, the first handle portion 362 and the second handle portion 364 are configured to be urged toward each other to cause the frame 310 to contract from a first configuration in which the first end 313 and the second end 315 of the frame 310 are a first distance apart to a second configuration in which the first end 313 and the second end 315 of the frame 310 are a second distance apart smaller than the first distance. Thus, the oral device 300 can be transitioned from the first configuration (as shown in FIG. 12) to the second configuration (as shown in FIG. 13) for insertion into the user’s mouth, and then allowed to transition from the second configuration to the first configuration when within the user’s mouth for engagement with the user’s teeth. As shown in FIG. 10, the first handle portion 362 and the second handle portion 364 can each include a plurality of grip portions (e.g., raised portions or ridges) disposed along the first handle portion 362 and the second handle portion 364 including the first grip portion 365A and the second grip portion 365B to assist the user in gripping the handle 360. The first notch 318A and the second notch 318B can assist the frame 310 in transitioning from the first configuration to the second configuration due to the frame 310 contracting (e.g., hinging) about an axis extending through the first notch 318A and the second notch 318B. In some embodiments, rather than the frame 310 contracting from the first configuration to the second configuration when the first handle portion 362 is urged toward the second handle portion 364, the frame 310 can expand from the second configuration to the first configuration when the first handle portion 362 is urged toward the second handle portion 364. In some embodiments, the frame 310 can be biased toward the first configuration. In some embodiments, the frame 310 can be biased toward the second configuration.

In some embodiments, the first wing 340, the first bite block 320, and a first end 313 portion of the frame 310 can collectively form a first channel 341 and a second channel 342 configured to receive a first set of upper teeth and a first set of lower teeth of the user, as shown in FIG. 11. The second wing 350, the second bite block 330, and a second end 315 portion of the frame 310 can collectively form a third channel 351 and a fourth channel 352 configured to receive a second set of upper teeth and a second set of lower teeth of the user, as shown in FIG. 11. As a result of the first set of upper teeth, the first set of lower teeth, the second set of upper teeth, and the second set of lower teeth being disposed in the first channel 341, the second channel 342, the third channel 351, and the fourth channel 352, respectively, the device 300 can be secured and maintained in the mouth of the user and/or can have limited movement relative to the teeth of the user (e.g., due to the user applying a compressive force to the first bite block 320 and the second bite block 330 with the user’s teeth).

As shown in FIGS. 10 and 11, the oral device 300 is symmetrical about a plane A including a portion of the frame 310, a portion of the first bite block 320, a portion of the second bite block 330, a portion of the first wing 340, a portion of the second wing 350, and a portion of the handle 360. The plane A can be an X-Z plane, as shown in FIGS. 10 and 11 (the Z-axis extending into and out of the page). The oral device 300 is also symmetrical about a plane B, which can be a Y-Z plane as shown in FIGS. 10 and 11. Thus, the oral device 300 can be inserted into the mouth of the user in one of two orientations. For example, the oral device 300 can be inserted such that the first bite block 320 is configured to be engaged by the left teeth of the user and the second bite block 330 is configured to be engaged by the right teeth of the user, and the oral device can be inserted such that the first bite block 320 is configured to be engaged by the right teeth of the user and the second bite block 330 is configured to be engaged by the left teeth of the user, eliminating the need for a user or a caregiver to determine the proper orientation of the oral device 300 before insertion into the user’s mouth.

In use, the oral device 300 can prevent the damage from teeth to teeth, teeth to cheek, teeth to lip, and/or teeth to tongue impact that can occur during biting or chewing (e.g., during a seizure). For example, during a seizure, damage to the teeth and/or lateral lacerations and/or other injuries to the cheek or tongue can be caused by the molars and/or canines. Due to the surface area ratio, the lower molars (i.e. molars and premolars) and canines can experience the most impact during the chewing motion. Additionally, lacerations and/or other injuries to the tip of the tongue can be caused by the incisors. The oral device 300 can prevent damage and injury to the teeth and tongue via the dissipation and mitigation of the force of impact between, for example, the top and bottom molars and canines and between the teeth and the tongue.

In some embodiments, the first bite block 320 and the second bite block 330 can each be sufficiently thick to absorb the force of the top teeth impacting the lower teeth such that the top teeth and the lower teeth are not damaged. In some embodiments, the first bite block 320 and the second bite block 330 can be shaped and sized such that the tongue (e.g., the tip of the tongue) is prevented from being disposed in the space between and simultaneously contacted by the upper teeth and the lower teeth with enough force to injure the tongue. For example, the first bite block 320 can be sufficiently thick between the first tooth engaging surface 322 of the first bite block 320 and the second tooth engaging surface 323 of the first bite block 320 and the second bite block 330 can be sufficiently thick between the first tooth engaging surface 332 of the second bite block 330 and the second tooth engaging surface 334 of the second bite block 330 such that a user cannot bite the tongue of the user when the oral device 300 is disposed in the mouth of the user due to the upper teeth and the lower teeth being prevented from simultaneously contacting the tongue or simultaneously contacting the tongue with enough force to injure the tongue.

In some embodiments, the oral device 300 can include flavoring materials. In some embodiments, the oral device 300 can be available in a variety of colors or combinations of colors. In some embodiments, the oral device 300 can include embossed or engraved designs.

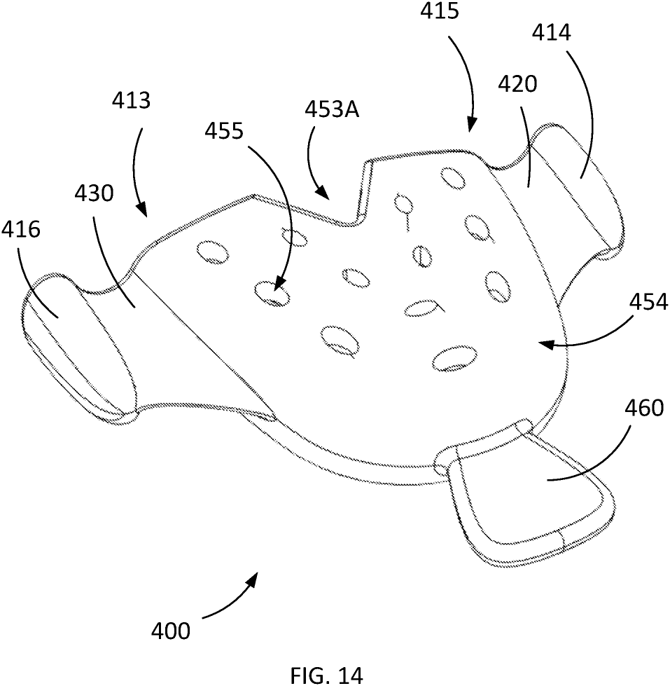

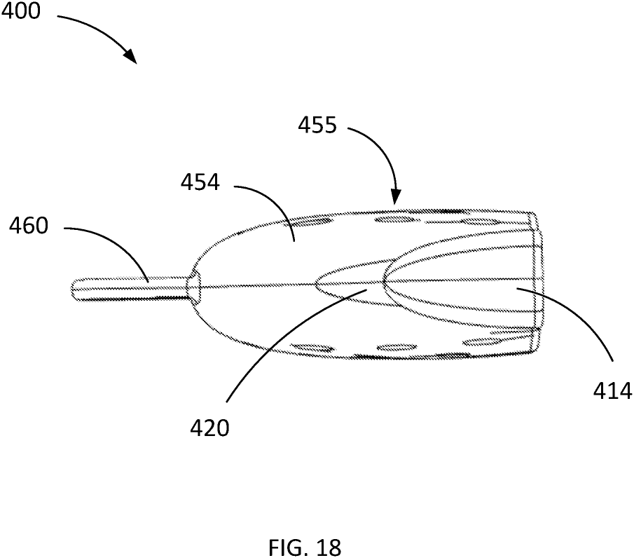

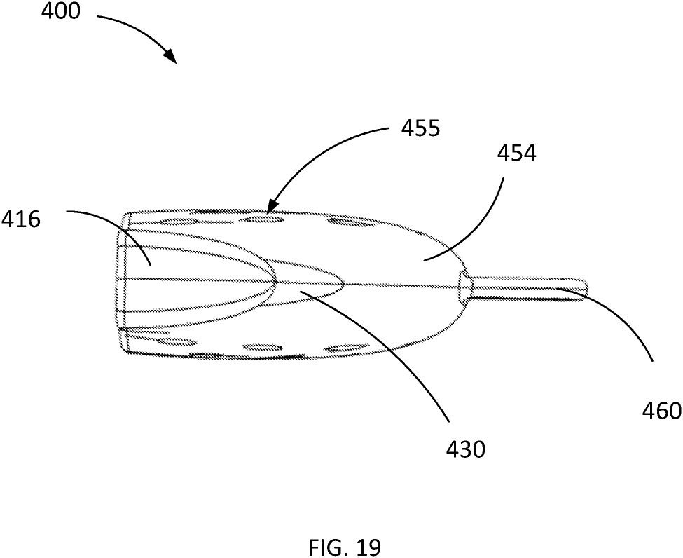

FIGS. 14-20 are front perspective, rear perspective, top, bottom, first side, second side, and rear views, respectively, of an oral device 400. The oral device 400 can be the same or similar in structure and/or function to any of the oral devices described herein, such as the oral device 100 and/or the oral device 300. For example, the oral device 400 includes a first bite block 420 and a second bite block 430 that can be the same or similar in structure and/or function to the first bite block 120, and the second bite block 130, respectively, described above with respect to FIG. 1. As shown in FIG. 14, the oral device includes a lingual frame 454 (also referred to as a “tongue protector” or a “tongue receptacle”) having a first end 413 and a second end 415 along a rear portion of the lingual frame 454. The first bite block 420 is coupled to the first end 413 of the lingual frame 454 and the second bite block 430 is coupled to the second end 415 of the lingual frame 454. In use, the oral device 400 can allow the user to swallow (e.g., swallow saliva) when the oral device 400 is disposed within the user’s mouth. The oral device 400 also includes a handle 460 that may be the same or similar in structure and/or function to the handle 160 described above with respect to the oral device 100.

The handle 460 extends distally from the lingual frame 454. The handle 460 can extend a sufficient distance from the lingual frame 454 such that the handle 460 protrudes from the user’s mouth when the oral device 400 is disposed within the mouth of the user. The handle 460 can be shaped as a tab. The handle 460 is configured to be gripped (e.g., by a user or caregiver) during insertion and positioning of the oral device 400 within the mouth of the user and/or during removal of the oral device 400 from the mouth of the user. In some embodiments, the handle 460 is not detachable from the curved frame 410 (e.g., the handle 460 is monolithically formed with the curved frame 410 or non-releasably engaged). In some embodiments, the handle 460 is removable from the curved frame 410 (e.g., via a tearaway portion including perforations). The handle 460 can be configured to maintain a separation between the user’s lips, thus allowing for oral fluids to be drawings and for breathing to be unobstructed through the distal opening 412.

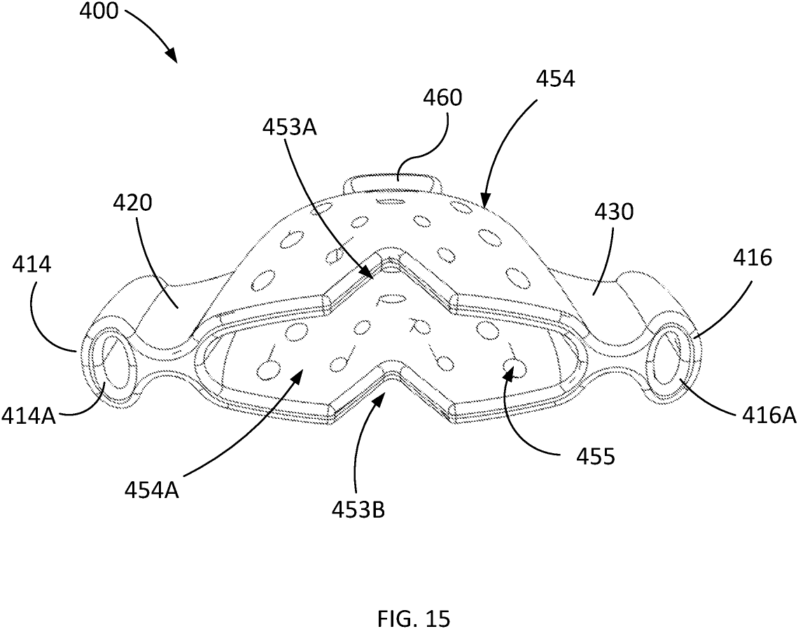

A first cheek protector 414 can be coupled to the first bite block 420 and a second cheek protector 416 can be coupled to the second bite block 430. The first cheek protector 414 can extend within or beyond the proximal and distal edges of the first bite block 430. The second cheek protector 416 can extend within or beyond the proximal and distal edges of the first bite block 430. As shown in FIG. 15, the first cheek protector 414 can define an interior space configured to receive a first support component 414A and the second cheek protector 416 can define an interior space configured to receive a second support component 416A. Each of the first support component 414A and the second support component 416A can be substantially cylindrical, elongated, biased toward a tubular shape, and/or elastomeric.

The first cheek protector 414 and the second cheek protector 416 can each include a portion that tapers proximally to facilitate insertion of the oral device 400 into the user’s mouth and/or to correspond to the shape of the user’s mouth cavity. As shown in FIG. 15, for example, the lingual frame 454 can define a first notch 453A defined along a first rear edge of the lingual frame 454 and a second notch 453B defined along a second rear edge of the lingual frame 454 opposite the first rear edge. The each of the first notch 453A and the second notch 453B can be configured to receive the lingual frenulum of the user depending on the orientation of the oral device 400 within the mouth.

In some embodiments, as shown in FIG. 14, the lingual frame 454 can define one or more lingual apertures 455 that extend through the lingual frame 454. Fluid can flow through the lingual apertures to reduce the build-up of saliva in a user’s mouth which may occur as a result of excessive salivation. The lingual apertures 455 can be configured such that, when the oral device 400 is disposed within the user’s mouth, the lingual apertures allow fluid to flow from the tongue to the rest of the mouth cavity. The lingual apertures 455 can be round (e.g., ovular or circular). The lingual apertures 455 can be sufficiently large to provide access to a mouth of a user through the lingual apertures 455 when the oral device 400 is disposed in the mouth of the user. For example, the lingual apertures 455 can be configured to receive a portion of a catheter such that fluid can be transferred relative to the mouth of the user via the catheter (e.g., saliva can be suctioned from the mouth and/or medication can be delivered to the mouth). The lingual apertures 455 can be configured to function as breathing vents such that air can flow through the lingual apertures 455. The lingual apertures 455 (and/or portions of the lingual frame 454 forming the lingual apertures 455) can be configured to oscillate under the impact or force of teeth clenching caused by the user biting down on the oral device 400.

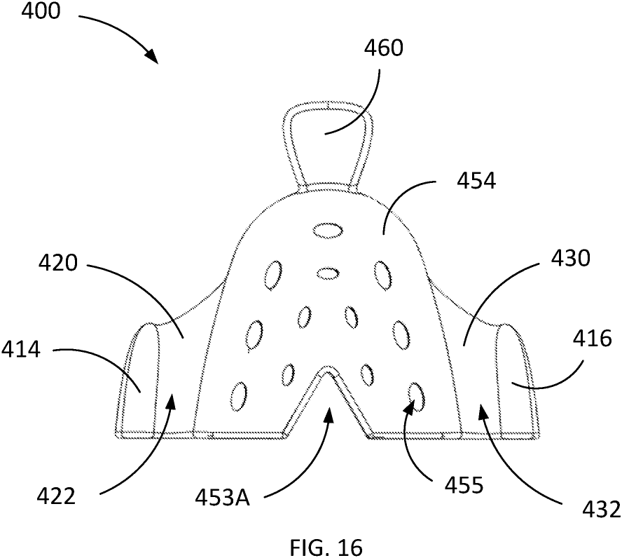

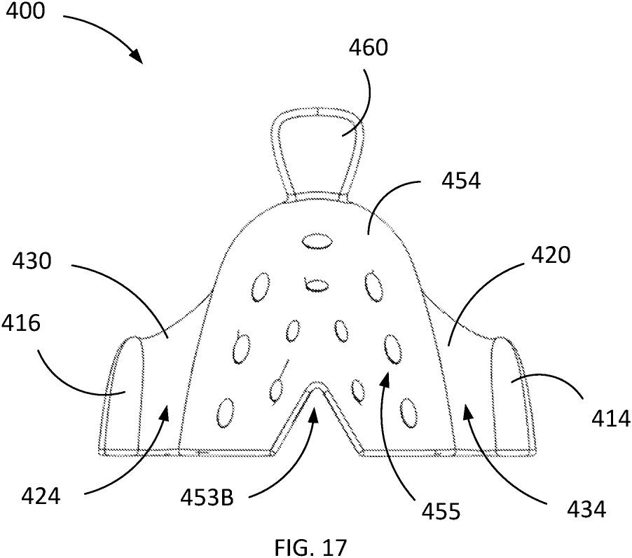

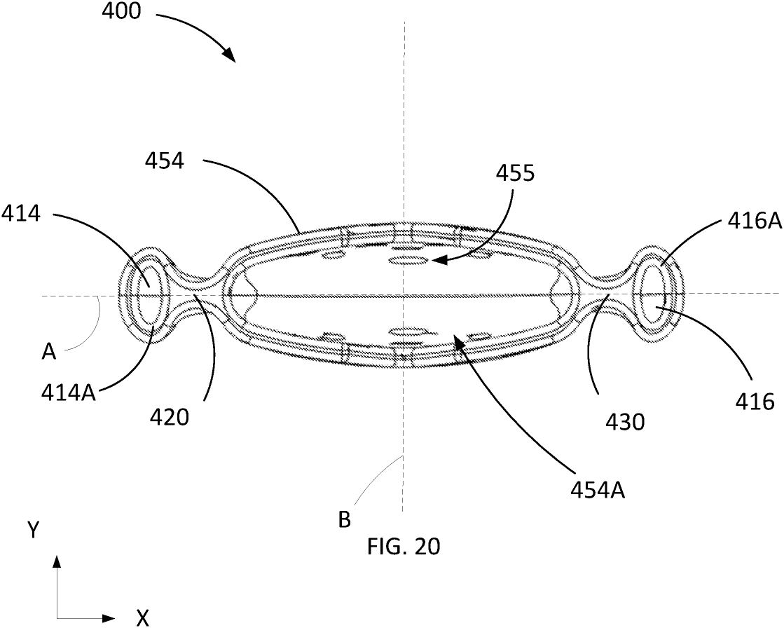

As shown in FIGS. 16-17, the first bite block 420 includes a first tooth engaging surface 422 and a second tooth engaging surface 424 opposite the first tooth engaging surface 422 and the second bite block 430 includes a first tooth engaging surface 432 and a second tooth engaging surface 434 opposite the first tooth engaging surface 432. The first tooth engaging surface 422 of the first bite block 420 can be disposed parallel or non-parallel to the second tooth engaging surface 424 of the first bite block 420. The first tooth engaging surface 432 of the second bite block 430 can be disposed parallel or non-parallel to the second tooth engaging surface 434 of the second bite block 430. As shown in FIGS. 15 and 20, for example, each of the first tooth engaging surface 422, the second tooth engaging surface 424, the first tooth engaging surface 432, and the second tooth engaging surface 434 can be curved such that a curved tooth receiving channel is defined by each of the first tooth engaging surface 422, the second tooth engaging surface 424, and the first tooth engaging surface 432. As shown in FIG. 16, the first bite block 420 and the second bite block 430 can extend distally of the first cheek protector portion 414 and the second cheek protector portion 416, respectively. For example, the first bite block 420 can extend from a distal end of the first cheek protector portion 414 to a first portion of a sidewall of the lingual frame 454 that is distal of the distal end of the first cheek protector portion 414. The second bite block 430 can extend from a distal end of the second cheek protector portion 416 to a second portion of a sidewall of the lingual frame (opposite the first portion) that is distal of the distal end of the second cheek protector portion 416.

The first bite block 420 and the second bite block 430 can be configured (e.g. shaped and sized) to be contacted by any suitable tooth or range of teeth. For example, in some embodiments, the first bite block 420 and the second bite block 430 can be shaped and sized such that the first bite block 420 and the second bite block 430 can be disposed between and contacted by the left and right upper and lower molars (e.g., the first and second premolars and first and second molars as shown in FIG. 4) and canines of the user, respectively. Said another way, the first bite block 420 and the second bite block 430 can be disposed between and in contact with teeth on the upper and lower left and right side of a user’s mouth, respectively, leaving the user’s incisors non-contacted. In some embodiments, the first bite block 420 and the second bite block 430 can be shaped to be contacted by the user’s range of teeth from the upper and lower canine to the upper and lower third molar (if the user has a lower third molar) on each side of the user’s mouth. In some embodiments, the first bite block 420 and the second bite block 430 can be shaped and sized to be disposed in contact with only the upper and lower molars or the upper and lower canines.

In some embodiments, the first bite block 420 and the second bite block 430 can be formed of a material with a high compressive strength to resist compression by the teeth during a biting or chewing movement. In some embodiments, the first bite block 420 and the second bite block 430 can be formed of an elastomeric mesh material. In some embodiments, the first bite block 420 and the second bite block 430 can have a higher elasticity than the frame 410.

The lingual frame 454 can be configured to corral the tongue of a user such that the tongue of the user is restrained from contacting the first tooth engaging surface 422 and the second tooth engaging surface 424 of the first bite block 420 or the first tooth engaging surface 432 and the second tooth engaging surface 434 of the second bite block 430 when the oral device 400 is disposed in a mouth of the user. As shown, for example, in FIG. 15, the lingual frame 454 is configured to define a concave receiving area 454A to receive the distal end of a user’s tongue and forms a boundary of movements of the tongue within the lingual frame 454.

As shown in FIG. 20, the oral device 400 is symmetrical about a plane A including a portion of the lingual frame 454, a portion of the first bite block 420, a portion of the second bite block 430, a portion of the first cheek protector 414, a portion of the second cheek protector 416. The plane A can be an X-Z plane (the Z-axis extending into and out of the page), as shown in FIG. 20. The oral device 400 is also symmetrical about a plane B, which can be a Y-Z plane as shown in FIG. 20. Thus, the oral device 400 can be inserted into the mouth of the user in one of two orientations. For example, the oral device 400 can be inserted such that the first bite block 420 is configured to be engaged by the left teeth of the user and the second bite block 430 is configured to be engaged by the right teeth of the user, and the oral device can be inserted such that the first bite block 420 is configured to be engaged by the right teeth of the user and the second bite block 430 is configured to be engaged by the left teeth of the user, eliminating the need for a user or a caregiver to determine the proper orientation of the oral device 400 before insertion into the user’s mouth.

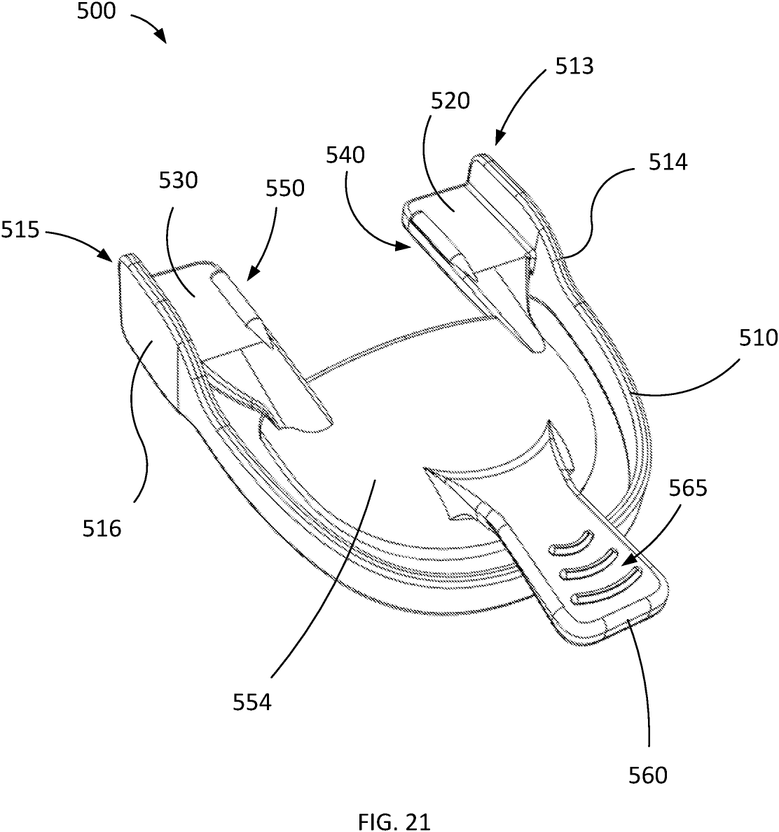

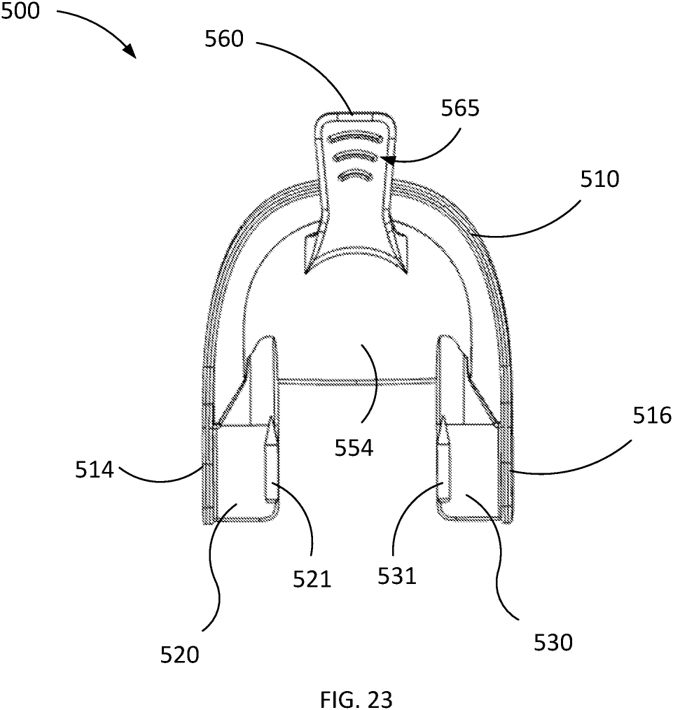

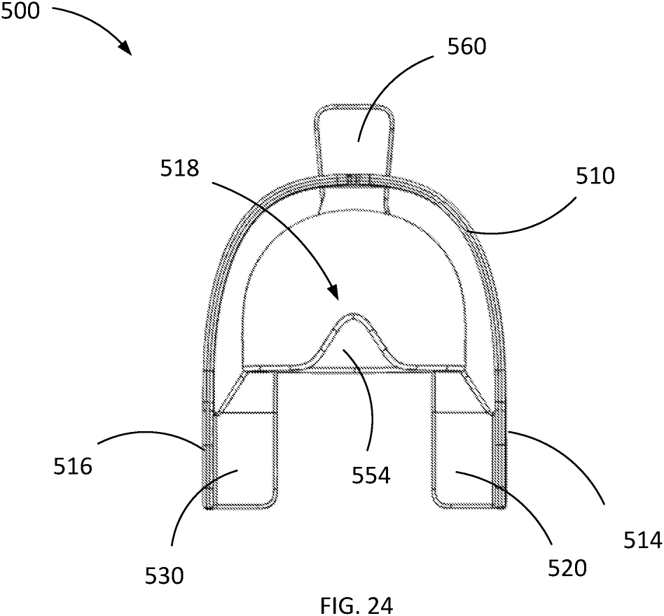

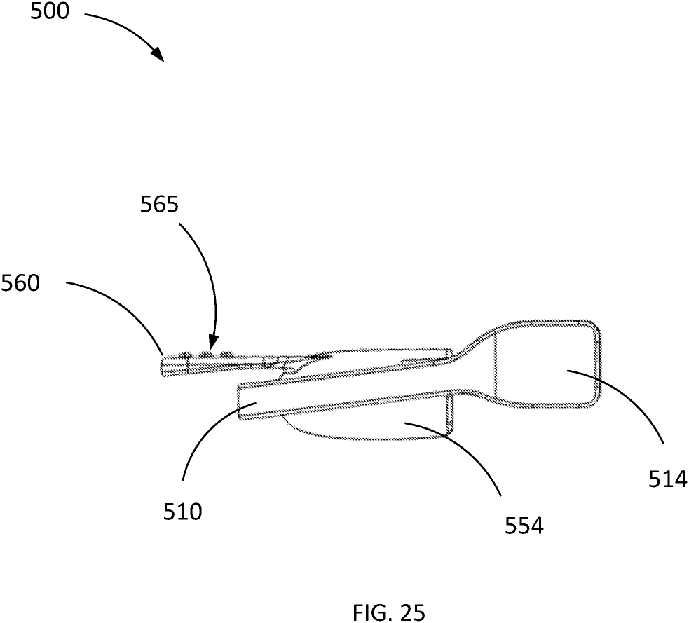

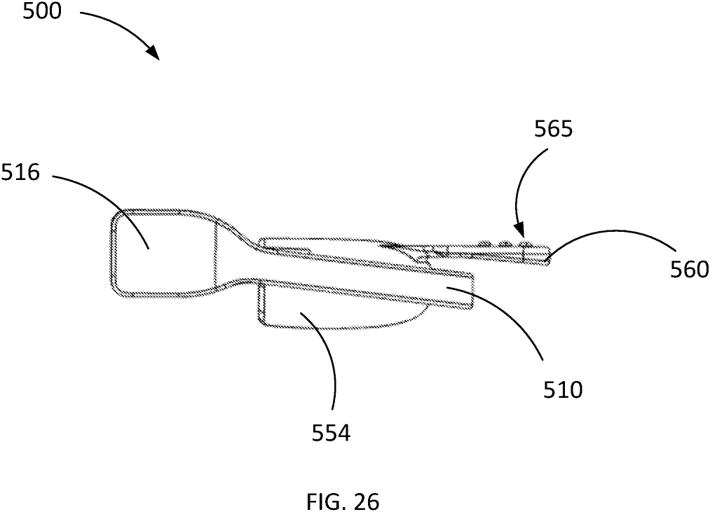

FIGS. 21-27 are front perspective, rear perspective, top, bottom, first side, second side, and rear views, respectively, of an oral device 500. The oral device 500 can be the same or similar in structure and/or function to any of the oral devices described herein, such as the oral device 100 and/or the oral device 400. For example, the oral device 500 includes a curved frame 510, a first bite block 520 and a second bite block 530 that can be the same or similar in structure and/or function to the curved frame 110, the first bite block 120, and the second bite block 130, respectively, described above with respect to FIG. 1. The first bite block 520 is coupled to the first end 513 of the curved frame 510 and the second bite block 530 is coupled to the second end 515 of the curved frame 510. The oral device 500 also includes a lingual frame 554 (also referred to as a “tongue protector” or a “tongue receptacle”) that can be the same or similar in structure and/or function to the lingual frame 454 described above with respect to FIG. 2. As shown in FIG. 23, the curved frame 110 is coupled to the lingual frame 554 via the first bite block 520 and the second bite block 530. In use, the oral device 500 can allow the user to swallow (e.g., swallow saliva) when the oral device 500 is disposed within the user’s mouth. The oral device 500 also includes a handle 560 that may be the same or similar in structure and/or function to the handle 160 described above with respect to the oral device 100 and/or the handle 460 described above with respect to the oral device 400.

The handle 560 extends distally from the lingual frame 554 and above the curved frame 510. The handle 560 can extend a sufficient distance from the lingual frame 554 such that the handle 560 protrudes from the user’s mouth when the oral device 500 is disposed within the mouth of the user. The handle 560 can be shaped as a tab and can include a number of protrusions or grips (e.g., two, three, four, or more) along a surface of the handle 560 to aid the user in gripping the handle 560 (e.g., to increase grip if the handle 560 has been contacted by saliva). The handle 560 is configured to be gripped (e.g., by a user or caregiver) during insertion and positioning of the oral device 500 within the mouth of the user and/or during removal of the oral device 500 from the mouth of the user.

The curved frame 510 can include a first cheek protector 514 disposed at a first end 513 of the curved frame 510 and a second cheek protector 516 disposed at a second end 515 of the curved frame 510. The first cheek protector 514 can be coupled to the first bite block 520 and the second cheek protector 516 can be coupled to the second bite block 530. The first cheek protector 514 can extend within or beyond the proximal and distal edges of the first bite block 530. The second cheek protector 516 can extend within or beyond the proximal and distal edges of the first bite block 530. As shown, for example, in FIG. 21, the first cheek protector 514 and the second cheek protector 516 can be wider (i.e., extend vertically higher and lower) than a remainder of the curved frame 510 and wider than the first bite block 520 and the second bite block 530, respectively. The first cheek protector 514 and the second cheek protector 516 can each include a portion that tapers distally toward a remainder of the curved frame 510. The curved frame 510 can form a barrier between the bottom teeth (e.g., lower jaw as shown in FIG. 4) and lower lip and extend along the outside arc of the bottom teeth (e.g., lower jaw as show in FIG. 4).

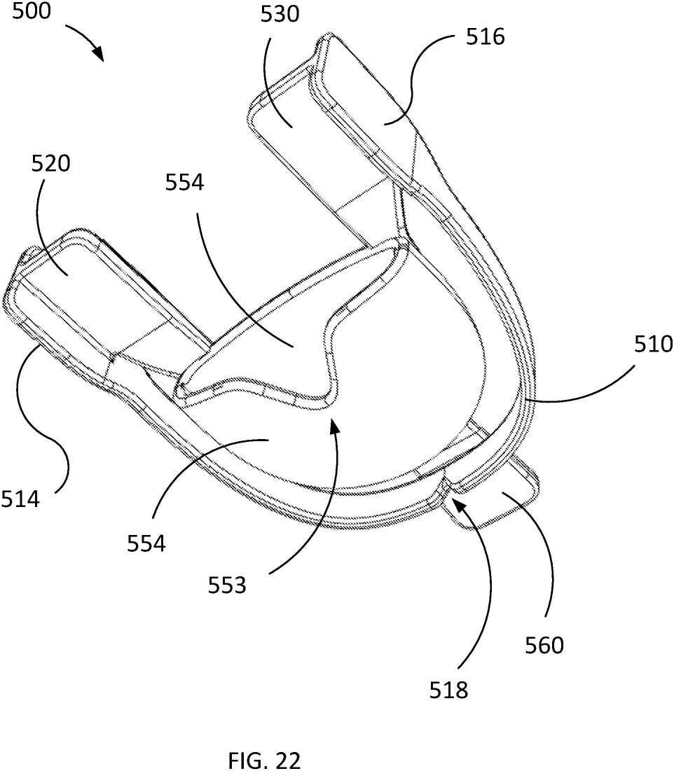

As shown in FIG. 22, for example, the lingual frame 554 can define a notch 453 defined along a lower rear edge of the lingual frame 554. The notch 553 can be configured to receive the lingual frenulum of the user. Although not shown, in some embodiments, the lingual frame 554 can define one or more lingual apertures 555 that extend through the lingual frame 554. Fluid can flow through the lingual apertures to reduce the build of saliva in a user’s mouth which may occur as a result of excessive salivation. The lingual apertures 555 can be configured such that, when the oral device 500 is disposed within the user’s mouth, the lingual apertures allow fluid to flow from the tongue to the rest of the mouth cavity. The lingual apertures 555 can be round (e.g., ovular or circular). The lingual apertures 555 can be configured to function as breathing vents such that air can flow through the lingual apertures 555. The lingual apertures 555 (and/or portions of the lingual frame 554 forming the lingual apertures 555) can be configured to oscillate under the impact or force of teeth clenching caused by the user biting down on the oral device 500.

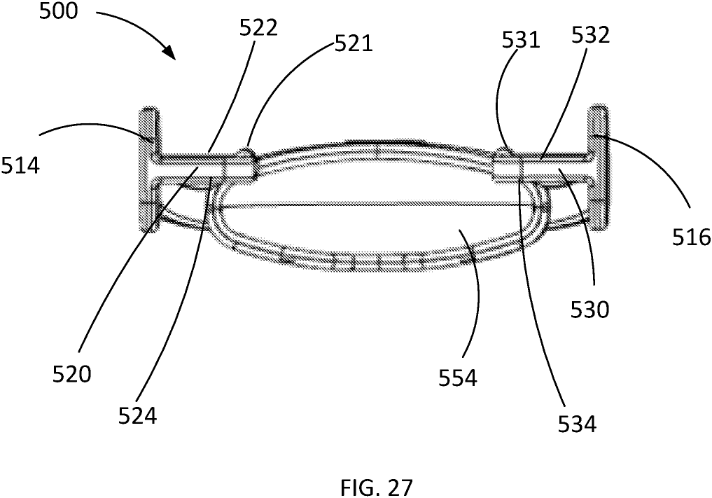

As shown in FIG. 27, the first bite block 520 includes a first tooth engaging surface 522 and a second tooth engaging surface 524 opposite the first tooth engaging surface 522 and the second bite block 530 includes a first tooth engaging surface 532 and a second tooth engaging surface 534 opposite the first tooth engaging surface 532. The first tooth engaging surface 522 of the first bite block 520 can be disposed parallel to the second tooth engaging surface 524 of the first bite block 520. The first tooth engaging surface 532 of the second bite block 530 can be disposed parallel to the second tooth engaging surface 534 of the second bite block 530. As shown in FIG. 23, the first bite block 520 can include or be coupled to a first elongated flange 521 and the second bite block 530 can include or be coupled to a second elongated flange 531. The first elongated flange 521 and the second elongated flange 531 can extend along an upper edge of the first bite block 520 and the second bite block 530, respectively. As shown in FIG. 23, each of the first elongated flange 521 and the second elongated flange 531 can include a tubular portion and a tapered or cone shaped portion that extends distally relative to the tubular portion. The first elongated flange 521 can define a boundary of a tooth-receiving channel defined by the first elongated flange 521, the first bite block 520, and the first cheek protector 514. The second elongated flange 531 can define a boundary of a tooth-receiving channel defined by the second elongated flange 531, the second bite block 530, and the second cheek protector 516. As shown in FIG. 21, for example, each of the first bite block 520 and the second bite block 530 can be coupled to the lingual frame 554 via extension portions such that the movement of the lingual frame 554 due to being coupled to the first bite block 520 and the second bite block 530.

The first bite block 520 and the second bite block 530 can be configured (e.g. shaped and sized) to be contacted by any suitable tooth or range of teeth. For example, in some embodiments, the first bite block 520 and the second bite block 530 can be shaped and sized such that the first bite block 520 and the second bite block 530 can be disposed between and contacted by the left and right upper and lower molars (e.g., the first and second premolars and first and second molars as shown in FIG. 4) and canines of the user, respectively. Said another way, the first bite block 520 and the second bite block 530 can be disposed between and in contact with teeth on the upper and lower left and right side of a user’s mouth, respectively, leaving the user’s incisors non-contacted. In some embodiments, the first bite block 520 and the second bite block 530 can be shaped to be contacted by the user’s range of teeth from the upper and lower canine to the upper and lower third molar (if the user has a lower third molar) on each side of the user’s mouth. In some embodiments, the first bite block 520 and the second bite block 530 can be shaped and sized to be disposed in contact with only the upper and lower molars or the upper and lower canines.

In some embodiments, the first bite block 520 and the second bite block 530 can be formed of a material with a high compressive strength to resist compression by the teeth during a biting or chewing movement. In some embodiments, the first bite block 520 and the second bite block 530 can be formed of an elastomeric mesh material. In some embodiments, the first bite block 520 and the second bite block 530 can have a higher elasticity than the frame 510.

The lingual frame 554 can be configured to corral the tongue of a user such that the tongue of the user is restrained from contacting the first tooth engaging surface 522 and the second tooth engaging surface 524 of the first bite block 520 or the first tooth engaging surface 532 and the second tooth engaging surface 534 of the second bite block 530 when the oral device 500 is disposed in a mouth of the user. As shown, for example, in FIG. 27, the lingual frame 554 is configured to define a concave receiving area 554A to receive the distal end of a user’s tongue and forms a boundary of movements of the tongue within the lingual frame 554.

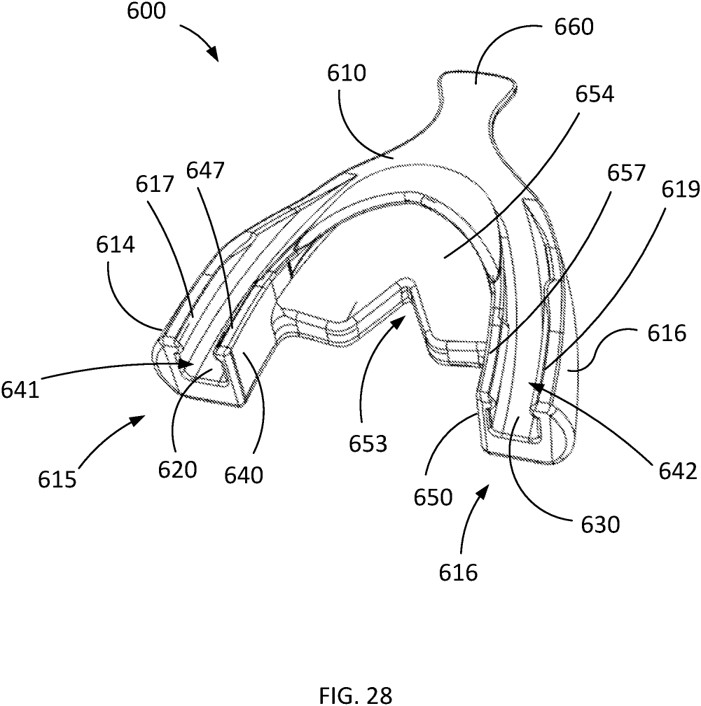

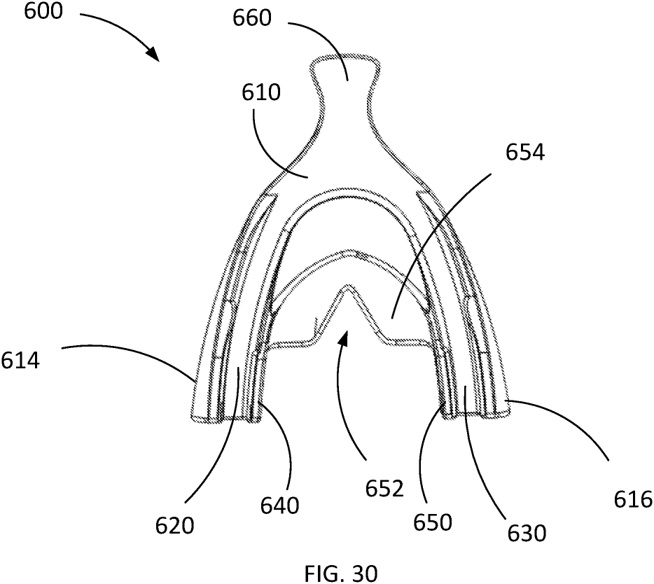

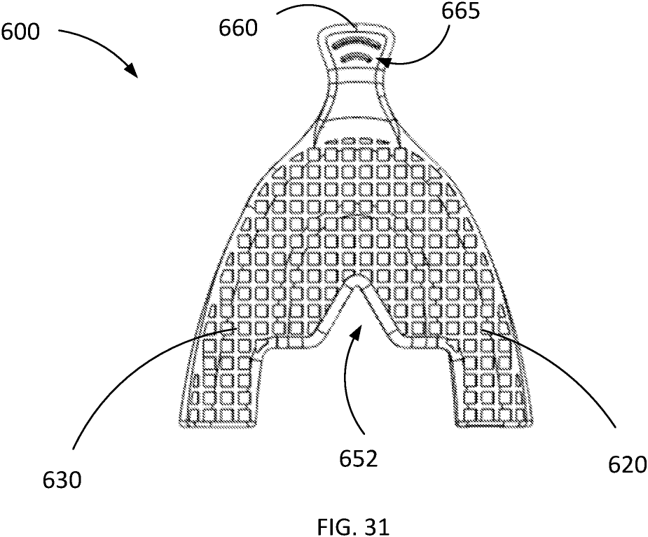

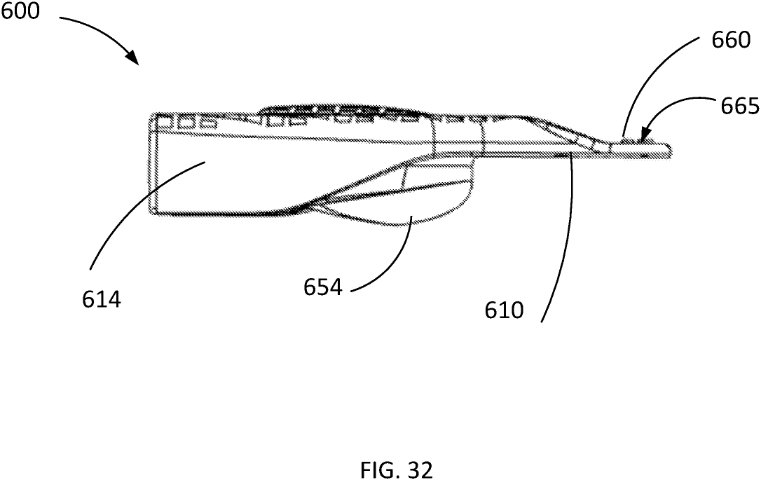

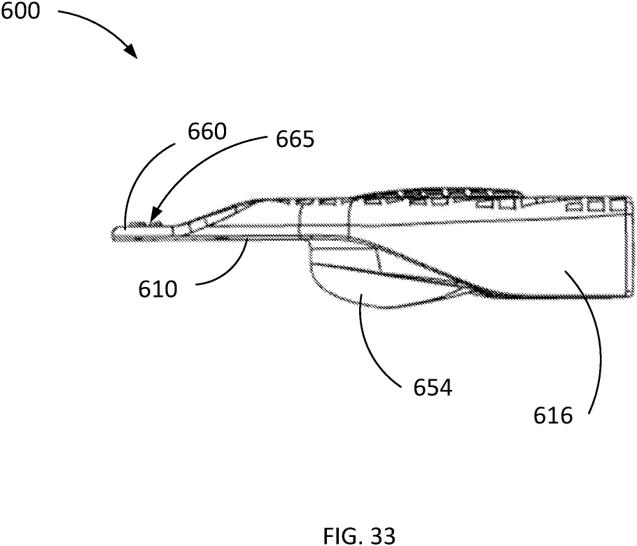

FIGS. 28-34 are front perspective, rear perspective, bottom, top, first side (i.e., right side), second side (i.e., left side), and rear views, respectively, of an oral device 600. The oral device 600 can be the same or similar in structure and/or function to any of the oral devices described herein, such as the oral device 100 and/or the oral device 400. For example, the oral device 600 includes a curved frame 610, a first bite block 620 and a second bite block 630 that can be the same or similar in structure and/or function to the curved frame 110, the first bite block 120, and the second bite block 130, respectively, described above with respect to FIG. 1. As shown in FIG. 28, for example, the curved frame 610, the first bite block 620, and the second bite block 630 can be monolithically formed as one continuous curved structure. The oral device 600 also includes a lingual frame 654 (also referred to as a “tongue protector” or a “tongue receptacle”) that can be the same or similar in structure and/or function to the lingual frame 454 described above with respect to FIG. 14. As shown in FIG. 28, the curved frame 610 is coupled directly to the lingual frame 654 such that the curved frame 610 forms a perimeter of an interior of the lingual frame 654. For example, the lingual frame 654 can include an upper portion and a lower portion coupled to an upper inner edge and a lower inner edge of the curved frame 610, respectively, such that the upper portion, lower portion, and curved frame 610 form an interior or pocket to receive a user’s tongue. In use, the oral device 600 can allow the user to swallow (e.g., swallow saliva) when the oral device 600 is disposed within the user’s mouth. The oral device 600 also includes a handle 660 that may be the same or similar in structure and/or function to the handle 160 described above with respect to the oral device 100 and/or the handle 460 described above with respect to the oral device 400.

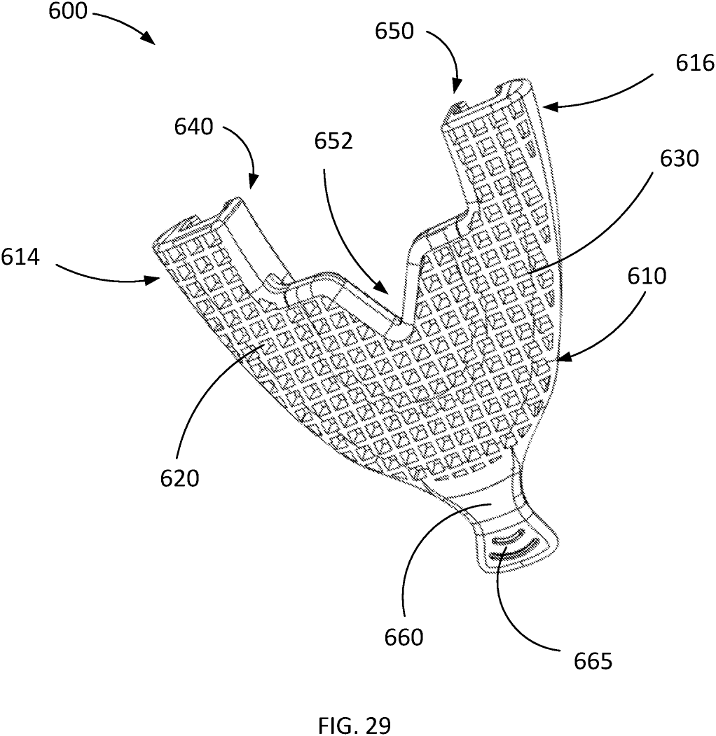

The handle 660 extends distally from the curved frame 610. The handle 660 can extend a sufficient distance from the curved frame 610 such that the handle 660 protrudes from the user’s mouth when the oral device 600 is disposed within the mouth of the user. The handle 660 can be shaped as a tab and can include a number of protrusions or grips 665 (e.g., two, three, four, or more) (shown, for example, in FIG. 29) along a surface of the handle 660 to aid the user in gripping the handle 660 (e.g., to increase grip if the handle 660 has been contacted by saliva). The handle 660 is configured to be gripped (e.g., by a user or caregiver) during insertion and positioning of the oral device 600 within the mouth of the user and/or during removal of the oral device 600 from the mouth of the user.

The curved frame 610 can include a first cheek protector 614 disposed at a first end 613 of the curved frame 610 and a second cheek protector 616 disposed at a second end 615 of the curved frame 610. The first cheek protector 614 can be coupled to the first bite block 620 and the second cheek protector 616 can be coupled to the second bite block 630. As shown, for example, in FIG. 28, the first cheek protector 614 and the second cheek protector 616 can be wider (i.e., extend vertically higher and lower) than a remainder of the curved frame 610 and wider than the first bite block 620 and the second bite block 630, respectively. The first cheek protector 614 and the second cheek protector 616 can each include a portion that tapers distally toward a remainder of the curved frame 610.

As shown in FIG. 28, for example, the lingual frame 654 can define a notch 653 defined along an upper rear edge of the lingual frame 654. As shown in FIG. 29, the upper surface of the oral device 600 can be formed of deformable mesh material. The deformable mesh material may include shape memory material. The deformable mesh material may be disposed over, for example, the upper surface of the lingual frame 654, the curved frame 610, the first bite block 620, and the second bite block 640. In other embodiments, the deformable mesh material may be disposed over, for example, the curved frame 610, the first bite block 620, and the second bite block 640.

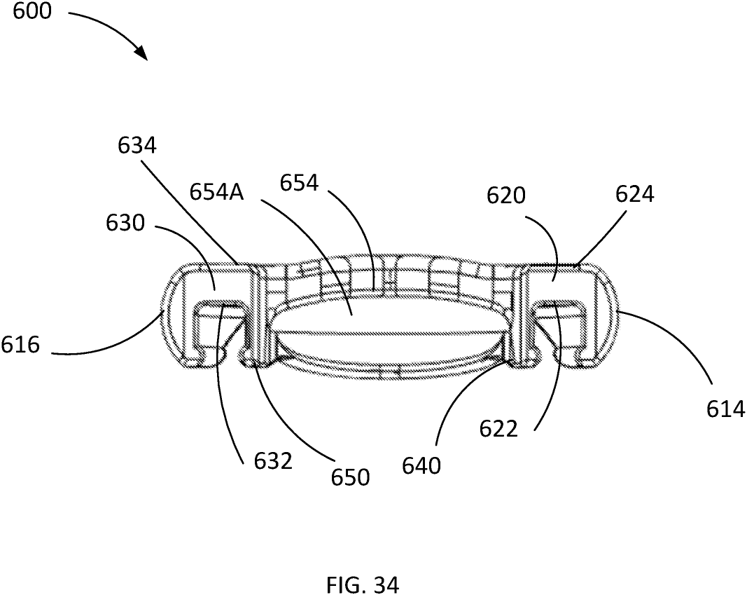

As shown in FIG. 34, the first bite block 620 includes a first tooth engaging surface 622 and a second tooth engaging surface 624 opposite the first tooth engaging surface 622 and the second bite block 630 includes a first tooth engaging surface 632 and a second tooth engaging surface 634 opposite the first tooth engaging surface 632. The first tooth engaging surface 622 of the first bite block 620 can be disposed parallel to the second tooth engaging surface 624 of the first bite block 620. The first tooth engaging surface 632 of the second bite block 630 can be disposed parallel to the second tooth engaging surface 634 of the second bite block 630.

The oral device 600 can include a first wing 640 and a second wing 650, which can be included in or formed monolithically with the curved frame 610. In some embodiments, The first wing 640 and the second wing 650 can form boundaries of the interior space of the lingual frame 654 and can taper distally. The first wing 640, the first bite block 620, and the first cheek protector 615 can collectively define a first channel 641 configured to receive a first set of lower teeth of the user. The second wing 650, the second bite block 630, and the second cheek protector 616 can collectively define a second channel 642 configured to receive a second set of lower teeth of the user.

As shown in FIG. 28, for example, the first cheek protector 614 can include a first elongated flange 617 and the first wing 640 can include a second elongated flange 647. The first elongated flange 617 and the second elongated flange 647 can be disposed parallel to each other. The second cheek protector 616 can include a second elongated flange 619 and the second wing 650 can include a second elongated flange 657. The second elongated flange 619 and the second elongated flange 657 can be disposed parallel to each other. The first elongated flange 617 and the second elongated flange 647 project into the channel 641 such that the first elongated flange 617 and the second elongated flange 647 can apply a frictional force to a set of teeth disposed in the first channel 641 to secure the oral device 600 relative to the set of teeth. The second elongated flange 619 and the second elongated flange 657 project into the channel 642 such that the first elongated flange 619 and the second elongated flange 657 can apply a frictional force to a set of teeth disposed in the second channel 642 to secure the oral device 600 relative to the set of teeth.

The first bite block 620 and the second bite block 630 can be configured (e.g. shaped and sized) to be contacted by any suitable tooth or range of teeth. For example, in some embodiments, the first bite block 620 and the second bite block 630 can be shaped and sized such that the first bite block 620 and the second bite block 630 can be disposed between and contacted by the left and right upper and lower molars (e.g., the first and second premolars and first and second molars as shown in FIG. 4) and canines of the user, respectively. Said another way, the first bite block 620 and the second bite block 630 can be disposed between and in contact with teeth on the upper and lower left and right side of a user’s mouth, respectively, leaving the user’s incisors non-contacted. In some embodiments, the first bite block 620 and the second bite block 630 can be shaped to be contacted by the user’s range of teeth from the upper and lower canine to the upper and lower third molar (if the user has a lower third molar) on each side of the user’s mouth. In some embodiments, the first bite block 520 and the second bite block 630 can be shaped and sized to be disposed in contact with only the upper and lower molars or the upper and lower canines.

In some embodiments, the first bite block 620 and the second bite block 630 can be formed of a material with a high compressive strength to resist compression by the teeth during a biting or chewing movement. In some embodiments, the first bite block 620 and the second bite block 630 can be formed of an elastomeric material. In some embodiments, the first bite block 620 and the second bite block 630 can have a higher elasticity than the frame 610.

The lingual frame 654 can be configured to corral a tongue of a user such that the tongue of the user is restrained within the interior of the lingual frame 654 from contacting the first tooth engaging surface 622 and the second tooth engaging surface 624 of the first bite block 630 or the first tooth engaging surface 632 and the second tooth engaging surface 634 of the second bite block 630 when the oral device 600 is disposed in a mouth of the user. As shown, for example, in FIG. 34, the lingual frame 654 is configured to define a concave receiving area 654A to receive the distal end of a user’s tongue and forms a boundary of movements of the tongue within the lingual frame 654.

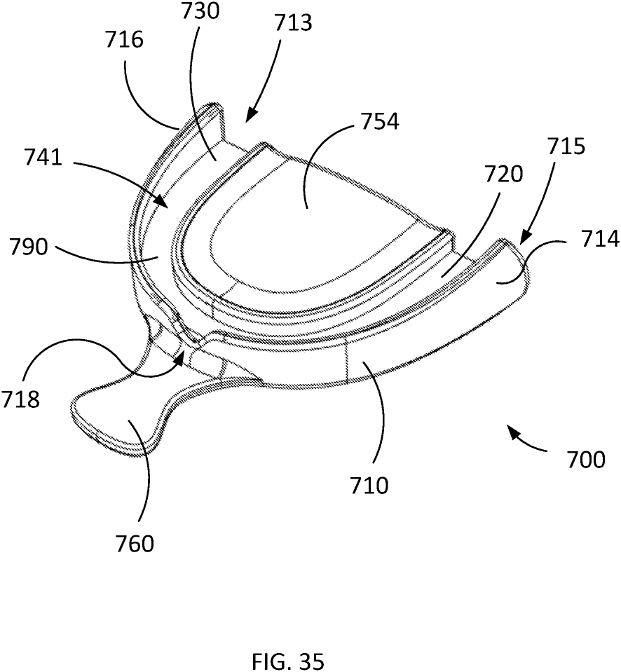

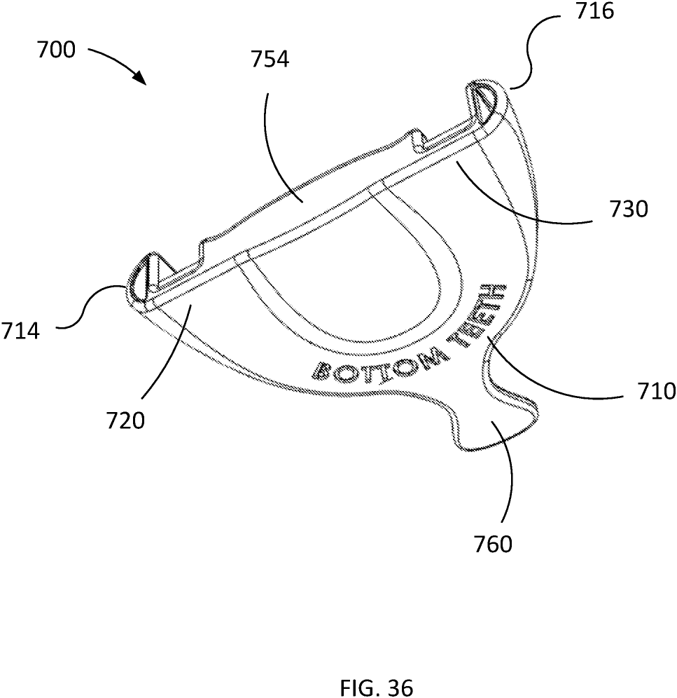

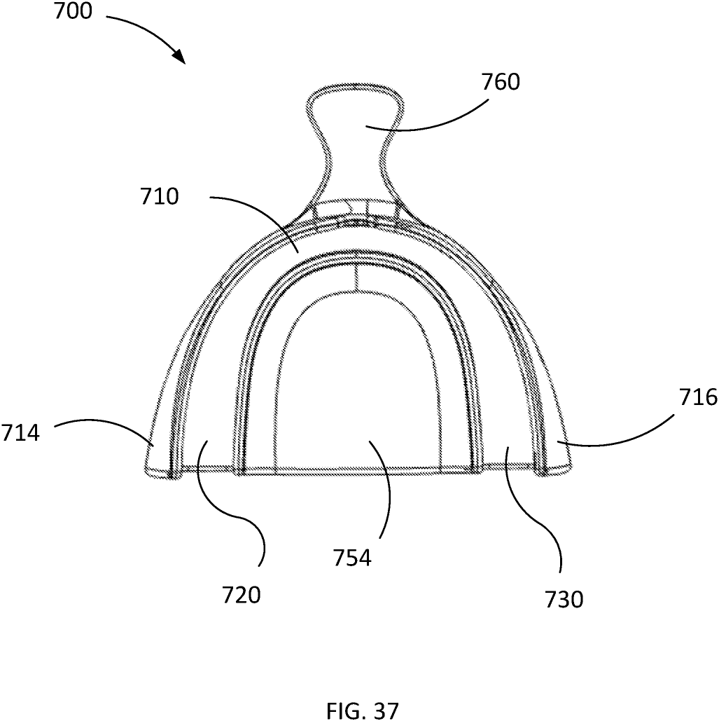

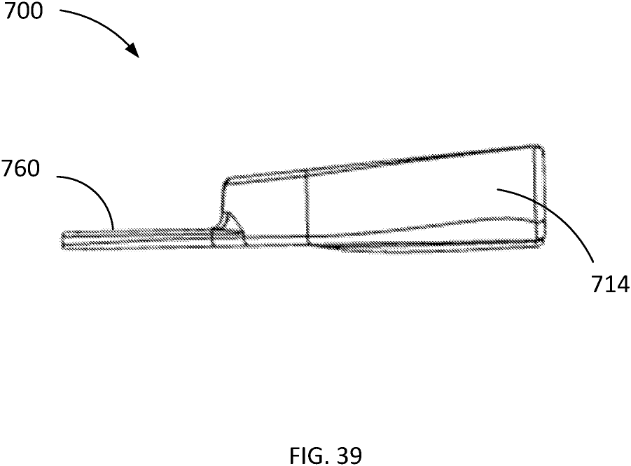

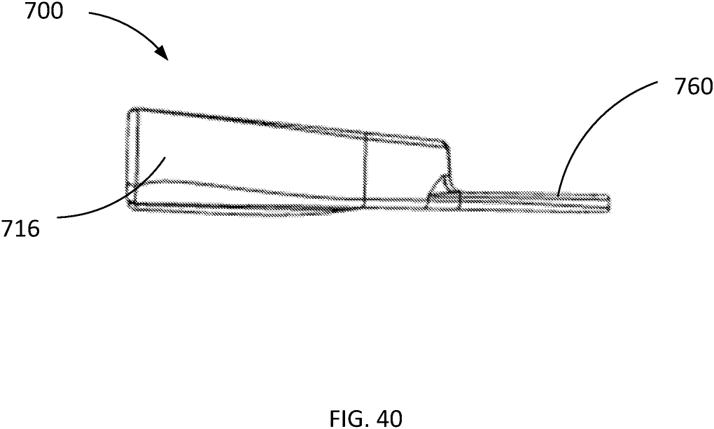

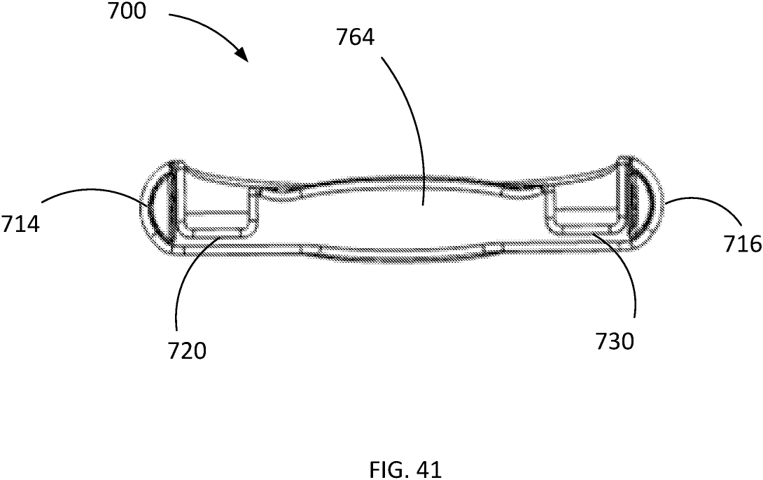

FIGS. 35-41 are front perspective, rear perspective, top, bottom, first side (i.e., left side), second side (i.e., right side), and rear views, respectively, of an oral device 700. The oral device 700 can be the same or similar in structure and/or function to any of the oral devices described herein, such as the oral device 100 and/or the oral device 600. For example, the oral device 700 includes a curved frame 710 that can be the same or similar in structure and/or function to the curved frame 110 respectively, described above with respect to FIG. 1. The oral device 700 can also include a first bite block 720 (also referred to as a first bite block portion or side) and a second bite block 730 (also referred to as a second bite block portion or side) opposite the first bite block portion. The first bite block 720 and the second bite block 730 can collectively form a continuous curved bite block configured to contact all of a user’s upper or lower teeth. As shown in FIG. 35, for example, the curved frame 710, the first bite block 720, and the second bite block 730 can be monolithically formed as one continuous curved structure. The oral device 700 also includes a lingual frame 754 (also referred to as a “tongue protector” or a “tongue receptacle”) that can be the same or similar in structure and/or function to any of the lingual frames described herein. As shown in FIG. 35, the curved frame 710 is coupled directly to the lingual frame 754 such that the curved frame 710 forms a perimeter of the lingual frame 754. For example, the lingual frame 754 can be formed as a substantially planar portion filling an interior space defined by the inner edge of the curved frame 710 such that a user’s tongue will be restricted to one side of the lingual frame 754 (e.g., a bottom side). The oral device 700 also includes a handle 760 that may be the same or similar in structure and/or function to the handle 160 described above with respect to the oral device 100 and/or the handle 460 described above with respect to the oral device 400.

The handle 760 extends distally from the curved frame 710. The handle 760 can extend a sufficient distance from the curved frame 710 such that the handle 760 protrudes from the user’s mouth when the oral device 700 is disposed within the mouth of the user. In use, the oral device 700 can allow the user to swallow (e.g., swallow saliva) when the oral device 700 is disposed within the user’s mouth. The handle 760 is configured to be gripped (e.g., by a user or caregiver) during insertion and positioning of the oral device 700 within the mouth of the user and/or during removal of the oral device 700 from the mouth of the user.

The curved frame 710 can include a first cheek protector 714 disposed at a first end 713 of the curved frame 710 and a second cheek protector 716 disposed at a second end 715 of the curved frame 710. The first cheek protector 714 can be coupled to the first bite block 720 and the second cheek protector 716 can be coupled to the second bite block 730. As shown, for example, in FIG. 35, the first cheek protector 714 and the second cheek protector 716 can be wider (i.e., extend vertically higher) than a remainder of the curved frame 710 and wider than the first bite block 720 and the second bite block 730, respectively. The first cheek protector 714 and the second cheek protector 716 can each include a portion that tapers distally toward a remainder of the curved frame 710. As shown in FIG. 35, the frame 710 can define a notch 718 defined along a first edge (e.g., an upper edge) of a center portion of the frame 710. The notch 718 can be configured to receive the upper frenulum of the user.

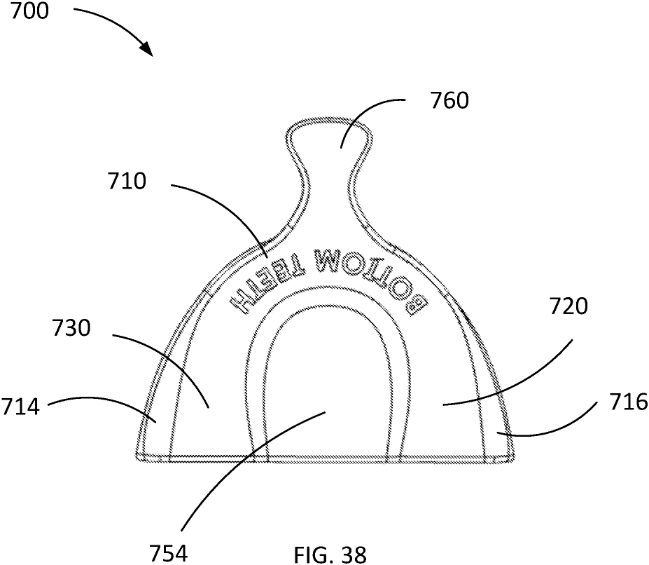

The curved frame 710 defines a curved channel 741 configured to receive the upper teeth of a user. In some embodiments, the curved frame 710 can include embossed or engraved wording, such as “BOTTOM TEETH” as shown in FIG. 38 to instruct the user on how to administer the device 700. The curved channel 741 can have a first open end adjacent the first end 713 of the curved frame 710 and a second open end adjacent the second end 715 of the curved frame 710. A retention material 790, such as a wax material, can be disposed within the curved channel 741. In some embodiments, the retention material 790 is hypoallergenic. The retention material 790 is configured to adhere to the upper teeth of the user to secure the oral device 700 in place relative to the user’s upper teeth. The oral device 700 can include a first molar receiving area 720 and a second molar receiving area 730 of the retention material 790 where the upper molars of the user are intended to be received by the channel 741 and the retention material 790.

The retention material 790 (and the channel 741) can be configured (e.g. shaped and sized) to be contacted by any suitable tooth or range of teeth. For example, in some embodiments, the retention material 790 can be configured to be contacted by all of the upper teeth of a user. In some embodiments, the retention material 790 can be configured to be contacted by all of the upper teeth of a user except for one or more rearmost teeth. In some embodiments, rather than being configured to receive and/or be coupled with the upper teeth of the user, the oral device 700 can be configured to receive and/or be coupled with the lower teeth (e.g., some or all of the bottom teeth) of the user (e.g., receive lower teeth in the curved channel 741 and depress the tongue of the user with the lingual frame 754).

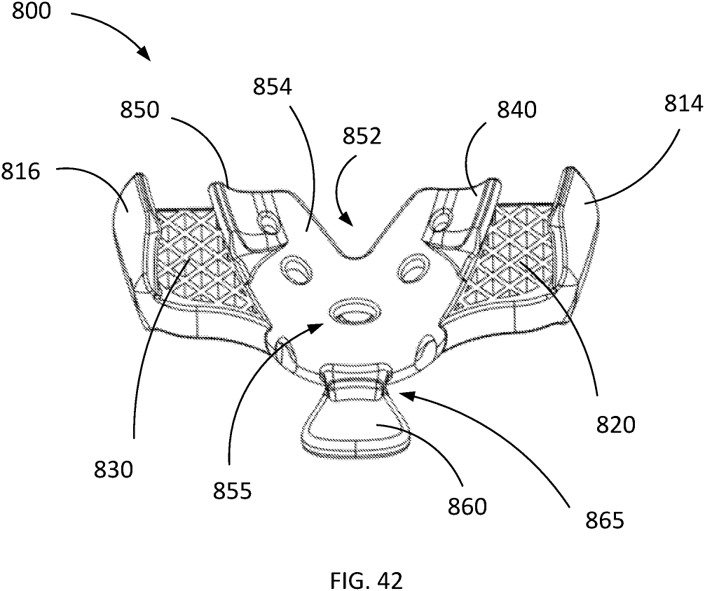

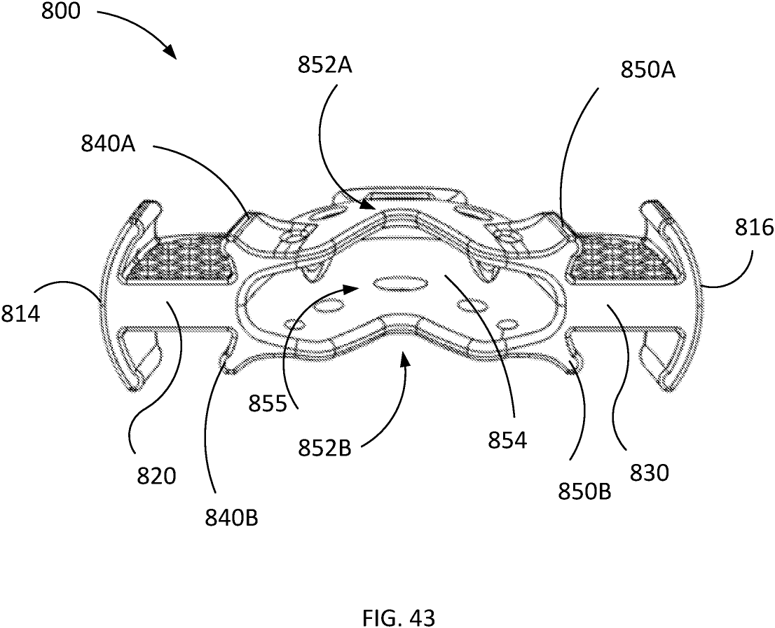

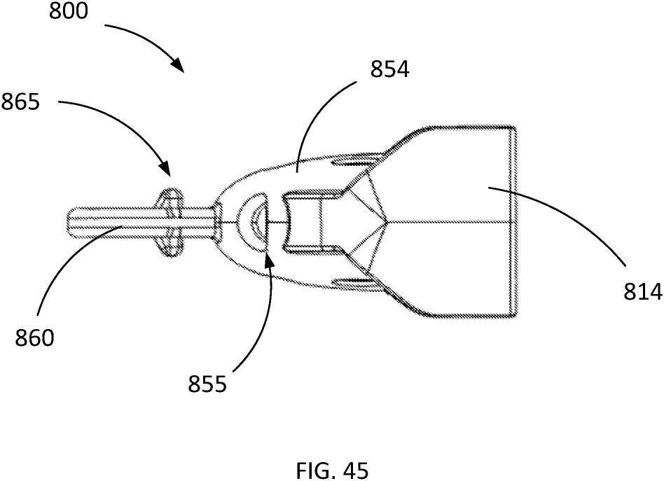

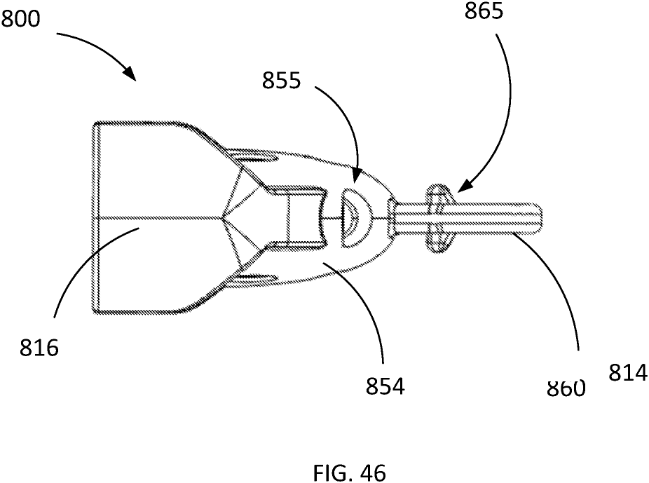

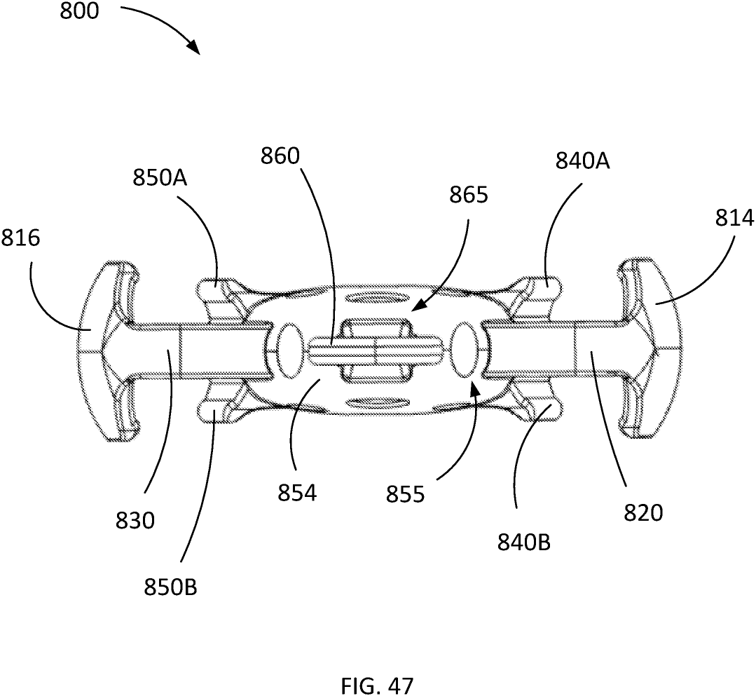

FIGS. 42-48 are front perspective, rear perspective, top, bottom, first side, second side, front, and rear views, respectively, of an oral device 800. The oral device 800 can be the same or similar in structure and/or function to any of the oral devices described herein, such as the oral device 100, the oral device 300, and/or the oral device 400. For example, the oral device 800 includes a first bite block 820 and a second bite block 830 that can be the same or similar in structure and/or function to the first bite block 120, and the second bite block 130, respectively, described above with respect to FIG. 1. As shown in FIG. 42, the oral device includes a lingual frame 854 (also referred to as a “tongue protector” or a “tongue receptacle”). The first bite block 820 is coupled to a first lateral side of the lingual frame 854 and the second bite block 830 is coupled to a second lateral side of the lingual frame 854. The oral device 800 also includes a handle 860 that may be the same or similar in structure and/or function to the handle 160 described above with respect to the oral device 100.

The handle 860 extends distally from the lingual frame 854. The handle 860 can extend a sufficient distance from the lingual frame 854 such that the handle 860 protrudes from the user’s mouth when the oral device 800 is disposed within the mouth of the user. The handle 860 can be shaped as a tab. The handle 860 is configured to be gripped (e.g., by a user or caregiver) during insertion and positioning of the oral device 800 within the mouth of the user and/or during removal of the oral device 800 from the mouth of the user. The handle 860 includes a stopper protrusion 865. The stopper protrusion 865 is configured be disposed against the outside of the lips of the user when the oral device 800 is disposed in the mouth of the user. A caregiver can user the stopper protrusion 865 to gauge how far to insert the oral device 800 into the user’s mouth.

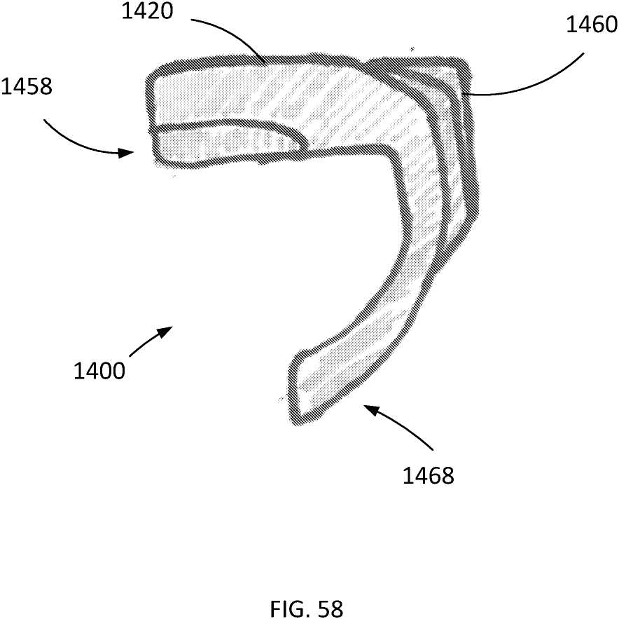

A first cheek protector 814 can be coupled to the first bite block 820 and a second cheek protector 816 can be coupled to the second bite block 830. The first cheek protector 814 can extend within the proximal and distal edges of the first bite block 830. The second cheek protector 816 can extend within the proximal and distal edges of the first bite block 830. As shown in FIG. 58, the first cheek protector 814 can be curved toward the first bite block 820 and the second cheek protector 816 can be curved toward the second bite block 830. The first cheek protector 814 and the second cheek protector 816 can each include a portion that tapers distally to correspond to the shape of the user’s mouth cavity.

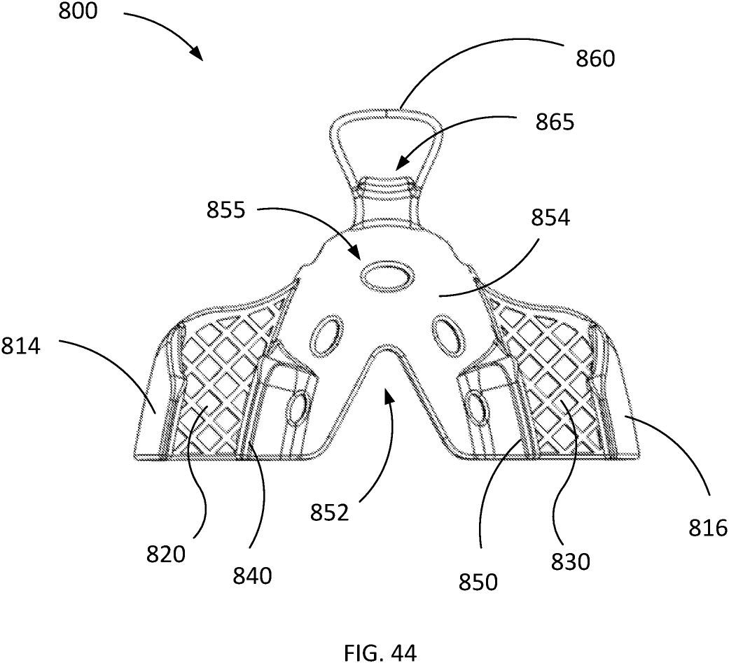

As shown in FIG. 44, the lingual frame 854 can define one or more lingual apertures 855 that extend through the lingual frame 854. Fluid can flow through the lingual apertures to reduce the build-up of saliva in a user’s mouth which may occur as a result of excessive salivation. The lingual apertures 855 can be configured such that, when the oral device 800 is disposed within the user’s mouth, the lingual apertures allow fluid to flow from the tongue to the rest of the mouth cavity. The lingual apertures 855 can be round (e.g., ovular or circular). The lingual apertures 855 can be configured to function as breathing vents such that air can flow through the lingual apertures 855. The lingual apertures 855 (and/or portions of the lingual frame 854 forming the lingual apertures 855) can be configured to oscillate under the impact or force of teeth clenching caused by the user biting down on the oral device 800. The oral device 800 can include more or fewer lingual apertures 855 than are depicted in FIGS. 42-48.

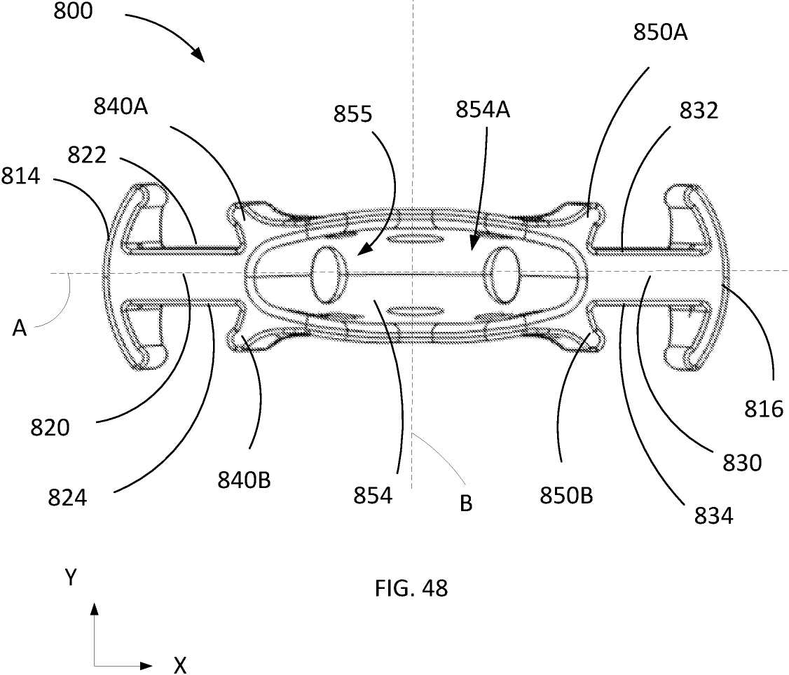

As shown in FIG. 48, the first bite block 820 includes a first tooth engaging surface 822 and a second tooth engaging surface 824 opposite the first tooth engaging surface 822 and the second bite block 830 includes a first tooth engaging surface 832 and a second tooth engaging surface 834 opposite the first tooth engaging surface 832. The first tooth engaging surface 822 of the first bite block 820 can be disposed parallel to the second tooth engaging surface 824 of the first bite block 820. The first tooth engaging surface 832 of the second bite block 830 can be disposed parallel to the second tooth engaging surface 834 of the second bite block 830.

The oral device 800 also includes a first wing 840 and a second wing 850 that can be the same or similar in structure and/or function any of the wings described herein. The first wing 840 is coupled to the first lateral side of the lingual frame 854 (e.g., along a top surface of the lingual frame 854 adjacent to a portion of the first bite block 820). The second wing 850 is coupled to the second lateral side of the lingual frame 854 (e.g., along a top surface of the lingual frame 854 adjacent to a portion of the second bite block 820). The first wing 840 can be curved toward the first bite block 820 and the first cheek protector 814 and the second wing 850 can be curved toward the second bite block 830 and the second cheek protector 816. In use, the oral device 800 can allow the user to swallow (e.g., swallow saliva) when the oral device 800 is disposed within the user’s mouth.

As shown in FIG. 44, the first bite block 820 and the second bite block 830 can extend distally of the first cheek protector portion 814 and the second cheek protector portion 816, respectively. For example, the first bite block 820 can extend from a distal end of the first cheek protector portion 814 to a first portion of a sidewall of the lingual frame 854 that is distal of the distal end of the first cheek protector portion 814. The second bite block 830 can extend from a distal end of the second cheek protector portion 816 to a second portion of a sidewall of the lingual frame (opposite the first portion) that is distal of the distal end of the second cheek protector portion 816.

The first bite block 820 and the second bite block 830 can be configured (e.g. shaped and sized) to be contacted by any suitable tooth or range of teeth. For example, in some embodiments, the first bite block 820 and the second bite block 830 can be shaped and sized such that the first bite block 820 and the second bite block 830 can be disposed between and contacted by the left and right upper and lower molars (e.g., the first and second premolars and first and second molars as shown in FIG. 4) and canines of the user, respectively. Said another way, the first bite block 820 and the second bite block 830 can be disposed between and in contact with teeth on the upper and lower left and right side of a user’s mouth, respectively, leaving the user’s incisors non-contacted. In some embodiments, the first bite block 820 and the second bite block 830 can be shaped to be contacted by the user’s range of teeth from the upper and lower canine to the upper and lower third molar (if the user has a lower third molar) on each side of the user’s mouth. In some embodiments, the first bite block 820 and the second bite block 830 can be shaped and sized to be disposed in contact with only the upper and lower molars or the upper and lower canines.

In some embodiments, the first bite block 820 and the second bite block 830 can be formed of a material with a high compressive strength to resist compression by the teeth during a biting or chewing movement. In some embodiments, the first bite block 820 and the second bite block 830 can be formed of an elastomeric mesh material. In some embodiments, the first bite block 820 and the second bite block 830 can have a higher elasticity than the frame 810. In some embodiments, the first bite block 820 and the second bite block 830 each define one or more perforations or apertures extending from the respective first tooth engaging surfaces 822, 832 to the respective second tooth engaging surfaces 824, 834. Fluid can flow through the apertures 826, 836 to reduce the buildup of saliva in a user’s mouth which may occur as a result of excessive salivation (e.g., during seizures). The apertures 826, 836 can be configured to function as breathing vents such that air can flow through the apertures 826, 836. The apertures 826, 836 (and/or portions of the first bite block 820 and the second bite block 830 forming the apertures 826, 836) can be configured to oscillate under the impact or force of teeth clenching caused by the user biting down on the oral device 800.

The lingual frame 854 can be configured to corral a tongue of a user such that the tongue of the user is restrained from contacting the first tooth engaging surface 822 and the second tooth engaging surface 824 of the first bite block 820 or the first tooth engaging surface 832 and the second tooth engaging surface 834 of the second bite block 830 when the oral device 800 is disposed in a mouth of the user. As shown, for example, in FIG. 48, the lingual frame 854 is configured to define a concave receiving area 854A to receive the distal end of a user’s tongue and forms a boundary of movements of the tongue within the lingual frame 854.

As shown in FIG. 48, the oral device 800 is symmetrical about a plane A including a portion of the lingual frame 854, a portion of the first bite block 820, a portion of the second bite block 830, a portion of the first cheek protector 814, a portion of the second cheek protector 816. The plane A can be an X-Z plane (the Z-axis extending into and out of the page), as shown in FIG. 48. The oral device 800 is also symmetrical about a plane B, which can be a Y-Z plane as shown in FIG. 48. Thus, the oral device 800 can be inserted into the mouth of the user in one of two orientations. For example, the oral device 800 can be inserted such that the first bite block 820 is configured to be engaged by the left teeth of the user and the second bite block 830 is configured to be engaged by the right teeth of the user, and the oral device can be inserted such that the first bite block 820 is configured to be engaged by the right teeth of the user and the second bite block 830 is configured to be engaged by the left teeth of the user, eliminating the need for a user or a caregiver to determine the proper orientation of the oral device 800 before insertion into the user’s mouth.

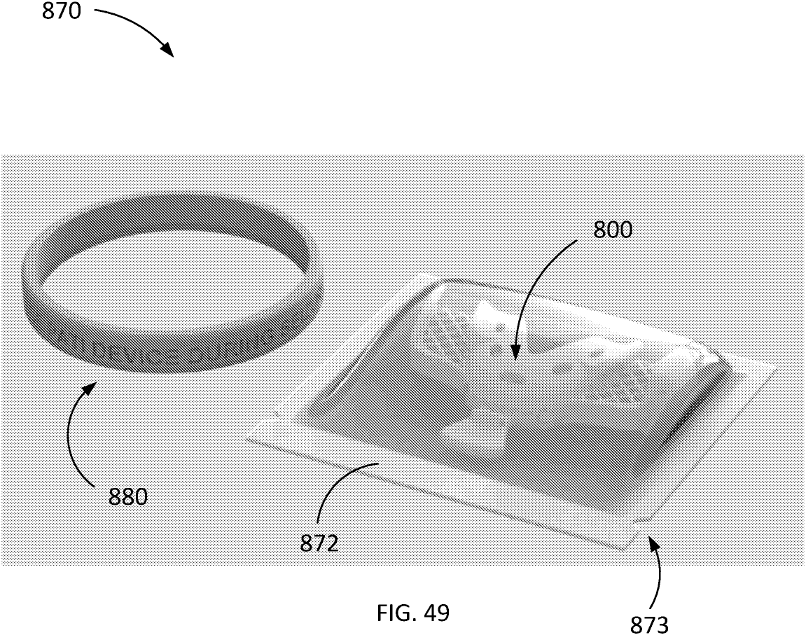

FIG. 49 is a front perspective view of a kit 870 including the oral device 800. As shown, the kit 870 can include a wristband 880 (also referred to as a bracelet) and a package 872 within which the oral device 800 can be disposed and maintained in a sterile environment. In some embodiments, the package 872 can be formed as a pouch. The package 872 defines notches 873 that can be used to facilitate tearing away a strip portion of the package 872 to access the interior of the package 872. In some embodiments, the package 872 may be transparent or partially transparent such that the oral device 800 is visible to a user and/or caregiver through the package 872. In some embodiments, the package 872 can be made of a sturdy foil material such that the oral device 800 is not visible through the packaging. In some embodiments, the package 872 can be labeled to include identification and/or instructions for use associated with the oral device 800. In some embodiments, the package 872 can include more than one oral device 800. In some embodiments, the package 872 can include Tyvek packaging, blister packaging, and/or any other suitable form of packaging. Even though the kit 870 is shown as including the oral device 800, in some embodiments the kit 870 can include any of the oral devices described herein. The wristband 880 can include a printed or molded message that can include a notification as to a medical status of the wearer and/or instructions regarding use of the oral device 800.

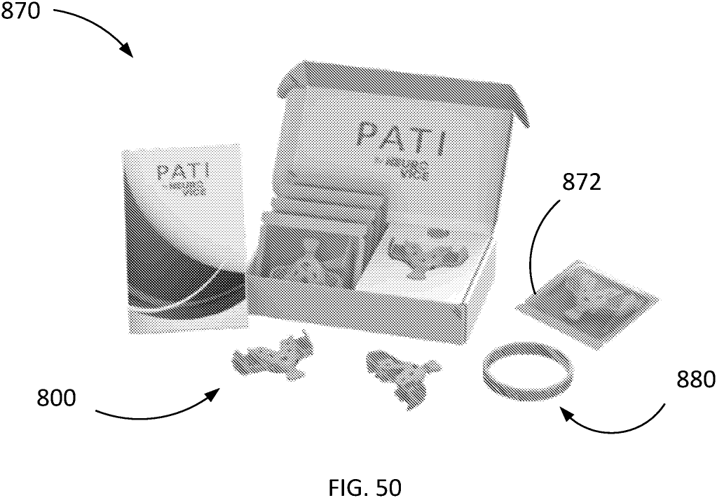

FIG. 50 is a front perspective view of the kit 870 with additional packages 872. As shown, a box package can include a plurality of kits 870, the bracelet 880, and/or an instruction manual. The kit 870 can be displayed as shown in FIG. 50 (e.g., in a clinic or hospital). In some embodiments, the kit 870 can be included as a prescription or prescription refill. In some embodiments, the box package can include a set of packages 872, each package 872 containing an oral device, such as any of the oral devices described herein. In some embodiments, the packages 872 can be stacked inside of a carton or box. In some embodiments, the kit 870 can be available as an over-the-counter or off-the-shelf product.

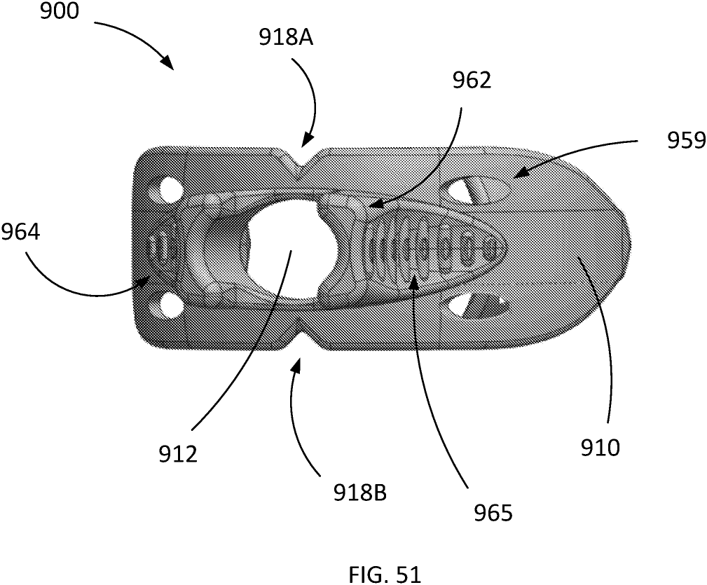

FIG. 51 is a front perspective view of an oral device 900. The oral device 900 can be the same or similar in structure and/or function to any of the oral devices described herein, such as the oral device 300. For example, the oral device 900 includes a curved frame 910, a handle having a first handle portion 962 and a second handle portion 964, a first notch 918A and a second notch 918B, and grip portions 965, which may be the same or similar in structure and/or function to similarly named components of the oral device 300. Additionally, the frame 910 defines a distal opening 912. As shown in FIG. 51, the oral device 900 also includes a number of openings 959 defined by the curved frame 910. Although FIG. 51 shows the oral device 900 shows four openings 959, the oral device 900 can define any suitable number of openings. In some embodiments, the openings 959 can allow for fluid (e.g., liquid and air) flow through the curved frame 910. In use, the oral device 900 can allow the user to swallow (e.g., swallow saliva) when the oral device 900 is disposed within the user’s mouth.

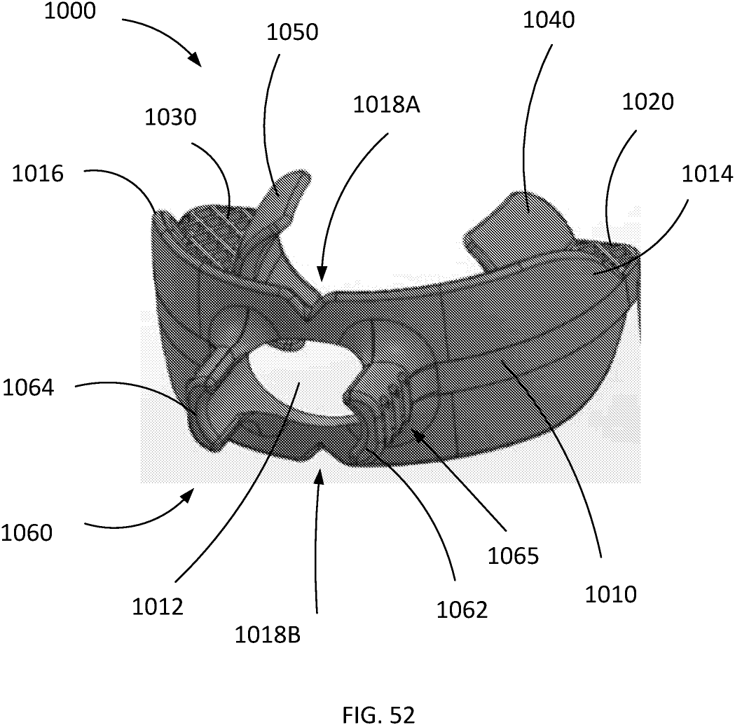

FIG. 52 is a front perspective view of an oral device 1000. The oral device 1000 can be the same or similar in structure and/or function to any of the oral devices described herein, such as the oral device 300. For example, the oral device 1000 includes a curved frame 1010, a handle 1060 having a first handle portion 1062 and a second handle portion 1064, a first notch 1018A and a second notch 1018B, and grip portions 1065, which may be the same or similar in structure and/or function to similarly named components of the oral device 300. Additionally, the frame 1012 defines a distal opening 1012. As shown in FIG. 52, the oral device 1000 also includes a first bite block 1020, a second bite block 1030, a first wing 1040, and a second wing 1050 that may be the same or similar in structure and/or function to similarly named components of the oral device 300. In use, the oral device 1000 can allow the user to swallow (e.g., swallow saliva) when the oral device 1000 is disposed within the user’s mouth. As shown in FIG. 52, the first handle portion 1062 and the second handle portion 1064 are discrete portions extending separately from the front surface of the curved frame 1010. Additionally, as shown in FIG. 52, the first bite block 1020 and the second bite block 1030 can extend farther proximally than cheek protector portions 1014, 1016 of the curved frame 1010, which taper toward the width of the first bite block 1020 and the second bite block 1030.

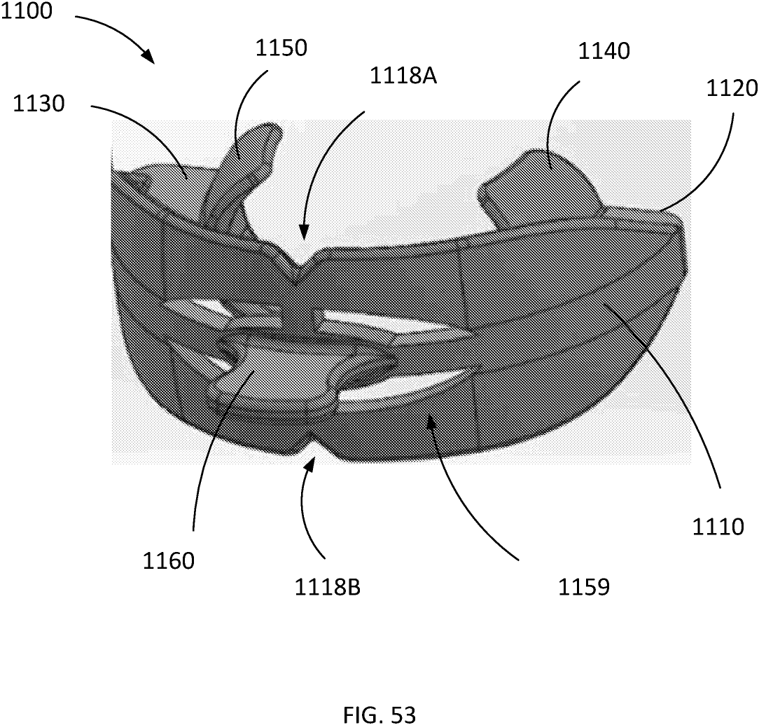

FIG. 53 is a front perspective view of an oral device 1100. The oral device 1100 can be the same or similar in structure and/or function to any of the oral devices described herein, such as the oral device 300 and the oral device 400. For example, the oral device 1100 includes a curved frame 1110, a handle 1160 having a tab portion extending distally from the curved frame 1110, a first notch 1118A, and a second notch 1118B, which may be the same or similar in structure and/or function to similarly named and/or described components of the oral device 300 and/or the oral device 400. Additionally, the oral device 1100 also includes a first bite block 1120, a second bite block 1130, a first wing 1140, and a second wing 1150 that may be the same or similar in structure and/or function to similarly named components of the oral device 300. As shown in FIG. 53, the oral device 1100 also includes a number of openings 1159 defined by the curved frame 1110 disposed near (e.g., partially surrounding) the handle 1160. The openings 1159 can allow for fluid (e.g., liquid and air) flow through the curved frame 1110. In some embodiments, the openings 1159 are sufficiently large such that a catheter can be inserted through the openings 1159 for fluid aspiration (e.g., suctioning) or delivery when the oral device 1100 is disposed in the mouth of the user. In use, the oral device 1100 can allow the user to swallow (e.g., swallow saliva) when the oral device 1100 is disposed within the user’s mouth.

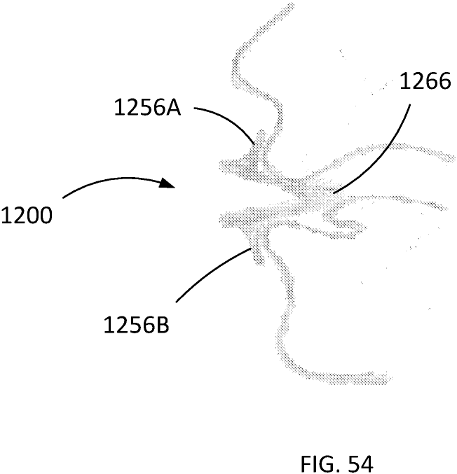

FIG. 54 is a side view of an oral device 1200. The oral device 1200 can be the same or similar in structure and/or function to any of the oral devices described herein, such as the oral device 100 and/or the oral device 300. In some embodiments, the oral device 1200 includes a hinge 1266. The hinge 1266 can be configured such that, when the oral device 1200 is disposed within the user’s mouth, the hinge 1266 allows for the oral device 1200 to open and close with the user’s mouth (i.e., allows the device to assume a closed configuration and open configuration). In some embodiments, the hinge 1266 includes sufficient stiffness and/or spring-like mechanisms to assist the oral device 1200 to return to an open configuration in the absence of sufficient force maintaining the oral device 1200 in a closed or partially closed configuration (i.e., the oral device 1200 can be biased toward the open configuration). In some embodiments, the oral device 1200 includes a first lip tab 1256A and/or a second lip tab 1256B. The first lip tab 1256A and/or second lip tab 1256B can be configured to receive and engage the upper and lower lips of the user. In use, the oral device 1200 can prevent accidental swallowing of the oral device 1200 due to the securement of the oral device 1200 to the mouth via the lip tabs 1256A, 1256B. The oral device 1200 can prevent accidental dislodging of the oral device 1200 due to the securement of the oral device 1200 to the teeth and mouth via the force applied by the hinge 1266. In use, the hinge 1266 allows the user to close their lips against the force of the hinge 1266 when the oral device 1200 is disposed within the user’s mouth, increasing comfort and decreasing excessive salivation. The oral device 1200 can be configured such that the tongue of the user is not engaged and/or constrained by the oral device 1200 when the oral device 1200 is disposed within the user’s mouth and in an open or closed configuration. In use, the oral device 1200 can allow ejection of matter through the oral cavity (e.g., vomit) and the ingestion of matter through the oral cavity (e.g., food and drink) (e.g., through one or more openings in the oral device 1200 and/or one or more openings collectively defined by an outer surface of the oral device and a portion of the user’s mouth). In use, the oral device 1200 can allow the user to swallow (e.g., swallow saliva) when the oral device 1200 is disposed within the user’s mouth.

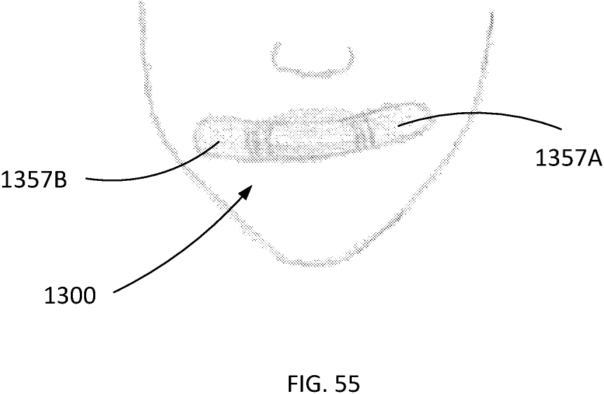

FIG. 55 is a front view of an oral device 1300. The oral device 1300 can be the same or similar in structure and/or function to any of the oral devices described herein, such as the oral device 100 and/or the oral device 100. In some embodiments, the oral device 1200 includes a first cheek tab 1357A and/or a second cheek tab 1357B. The first cheek tab 1357A and/or second cheek tab 1357B can be configured to engage the left and right cheeks of the user. In use, the oral device 1300 can prevent accidental swallowing of the oral device 1300 due to the securement of the oral device 1300 to the outside of the user’s face via the cheek tabs 1357A, 1357B. The oral device 1300 can be configured such that the tongue of user is not engaged and/or constrained by the oral device 1300 when the oral device 1300 is disposed within the user’s mouth and in an open or closed configuration. In use, the oral device 1300 can allow ejection of matter through the oral cavity (e.g., vomit) and the ingestion of matter through the oral cavity (e.g., food and drink) (e.g., through one or more openings in the oral device 1200 and/or one or more openings collectively defined by an outer surface of the oral device and a portion of the user’s mouth). In use, the oral device 1300 can allow the user to swallow (e.g., swallow saliva) when the oral device 1300 is disposed within the user’s mouth.

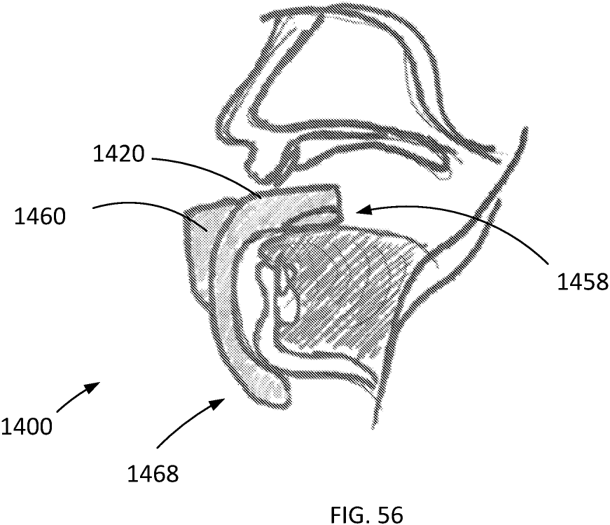

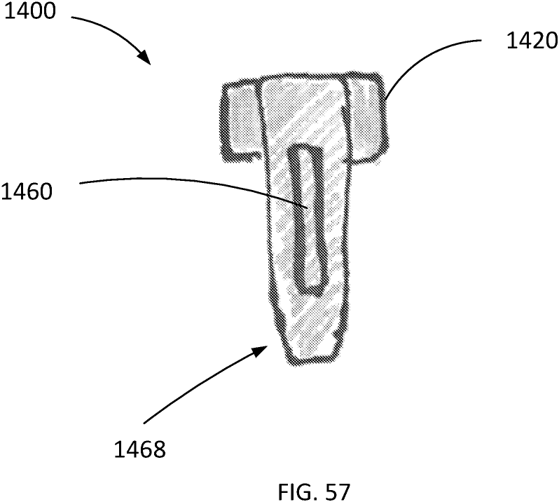

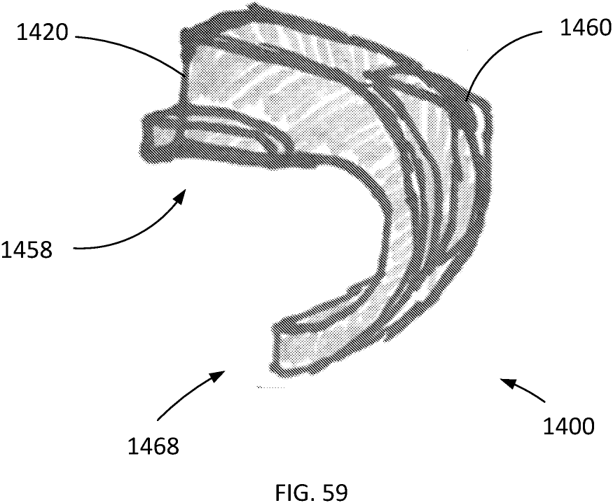

FIGS. 56 to 59 are side, front, opposite side, and perspective views, respectively, of an oral device 1400. The oral device 1400 can be the same or similar in structure and/or function to any of the oral devices described herein, such as the oral device 100 and/or the oral device 300. For example, the oral device can include a bite block 1420 configured to engage the surface of some or all of the teeth in a user’s upper and lower jaw when the oral device 1400 is disposed within the user’s mouth. Thus, the bite block 1420 can prevent teeth in the user’s lower jaw from contacting the teeth in the user’s upper jaw when the oral device 1400 is disposed within the user’s mouth. In some embodiments, the bite block 1420 is formed to engage the user’s incisors, canines, and/or premolars (i.e., the teeth disposed at the front of the mouth) when the oral device 1400 is disposed in the user’s mouth. In some embodiments, the oral device 1400 includes a handle 1460. A user or caregiver can insert the bite block 1420 into the user’s mouth and/or remove the bite block 1420 from the user’s mouth via grasping the handle 1460. In some embodiments, the oral device 1400 includes a tongue depressor 1458. The tongue depressor 1458 can be configured such that, when the oral device 1400 is disposed within the user’s mouth, the tongue depressor 1458 can apply pressure on the superior surface of a user’s tongue. In some embodiments, the tongue depressor 1458 is coupled to the bite block 1420. In some embodiments, the tongue depressor 1458 extends laterally and/or distally relative to an outer surface or perimeter of the bite block 1420. In some embodiments, the oral device 1400 includes a chin hook 1468. The chin hook 1468 can be configured to receive and engage the anterior and/or inferior sides of the chin of the user (e.g., during the insertion of the bite block 1420 into the user’s mouth). In use, the oral device 1400 can prevent accidental swallowing of the oral device 1400 due to the securement of the oral device 1400 to the outside of the user’s face (e.g., the user’s chin) via the chin hook 1468. The oral device 1400 can prevent accidental dislodging of the oral device 1400 due to the securement of the oral device 1400 to the mouth via the tongue depressor 1458 and/or via the chin hook 1468.

In some embodiments, the chin hook 1468 can be repositioned (e.g., shortened, extended from, and/or rotated) relative to the bite block 1420 to accommodate a user’s chin. In some embodiments, the chin hook 1468 includes an internal hinge mechanism that allows the oral device 1400 to fold together when the oral device 1400 is not disposed in the user’s mouth (e.g., for storage).

In some embodiments, the tongue depressor 1458 is formed as or includes a cantilever extending from the curved frame 1410, and can have a sufficient stiffness at the base of the cantilever such that the cantilever prevents the user’s tongue from deflecting the tongue depressor 2458 away from the user’s lower jaw and prevents the user’s tongue from moving between the upper and lower teeth of the user. In some embodiments, the tongue depressor 1458 can be disposed such that it applies a downward force to the tongue of the user to restrain the tongue in the bottom of the user’s mouth. In some embodiments, the tongue depressor 1458 is coupled to the bite block 1420 such that when the oral device 1400 is disposed within the mouth of a user the tongue depressor 1458 is disposed between the user’s tongue and the bite block 1420.

In use, a caregiver or the user can grip the handle 1460 and insert the bite block 1420 into the mouth of the user. The chin hook 1468 can be engaged with the user’s chin simultaneously or subsequently to the bite block 1420 being inserted into the user’s mouth. The oral device 1400 can be secured to the user’s lower jaw such that the user can open and close their mouth (i.e., teeth in the user’s lower jaw remain engaged with the bite block 1420 but teeth in the user’s upper jaw can come into and out of contact with the bite block 1420 as the user opens and closes their mouth) with the bite block 1420 within the user’s mouth. In use, the oral device 1400 can allow the user to swallow (e.g., swallow saliva) when the oral device 1400 is disposed within the user’s mouth.

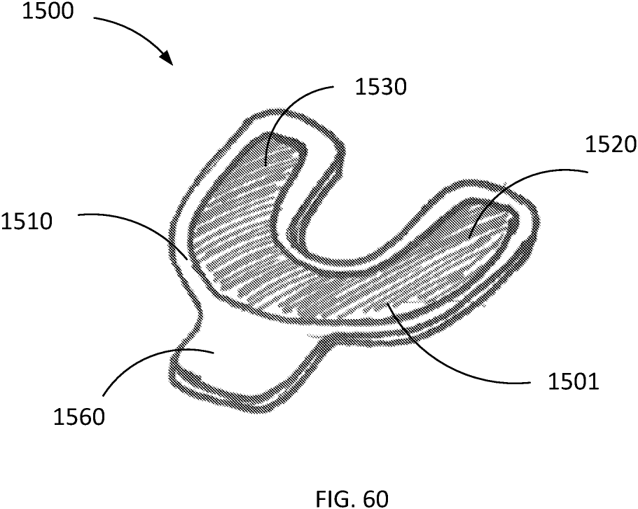

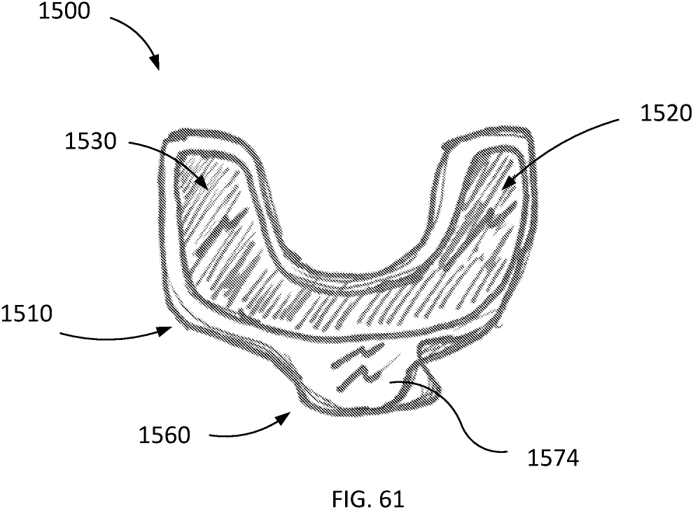

FIGS. 60 and 61 are front perspective views of an oral device 1500. The oral device 1500 can be the same or similar in structure and/or function to any of the oral devices described herein, such as the oral device 100 and/or the oral device 700. For example, the oral device 1500 includes a curved frame 1510 and a curved bite block 1501. The bite block 1501 includes a first bite block portion 1520 and second bite block portion 1530. The curved bite block 1501 is configured to contact all or all but one or more of the more proximal teeth of the upper and/or lower teeth of a user (e.g., molars). In some embodiments, the oral device 1500 includes a handle 1560 to facilitate insertion, removal, and positioning within the mouth of the user. In some embodiments, the curved frame 1510 is made of a rigid material and the curved bite block 1501 is made of a pliable retention material, such as a wax (e.g., a hypoallergenic wax such as dental wax) or a clay-like material. In some embodiments, the curved frame 1520 can be made of a rigid polymer (e.g., polypropylene, high density polyethylene (HDPE), and/or acrylic). In some embodiments, the oral device 1500 can be configured to be disposed within the mouth of the user for a single period of use, and then discarded after the single period of use. In some embodiments, the oral device 1500 includes a lid 1574 (e.g., a transparent lid or film) that is removable (e.g., from at least the retention material) prior to the oral device 1500 being disposed in the mouth of a user. The lid 1574 is made of a removable material that protects the pliable surfaces of the first and second bite block portions 1520, 1530 until the insertion of the oral device 1500 into the user’s mouth is required. The pliable retention material of the first and second bite blocks 1520, 1530 can be configured to receive the upper and/or lower teeth of the user. In some embodiments, the pliable retention material can be configured to be deformed by the user’s teeth when the oral device 1500 is disposed within the mouth of the user and the user bites the pliable retention material. As the user bites (e.g., down) into the pliable retention material, the pliable retention material can be deformed under the force of the user’s teeth to define depressions that complement the shape of the user’s teeth contacting the pliable retention material. The pliable retention material can be formed of any suitable material such that the pliable retention material can retain the user’s teeth within depressions in the pliable retention material when the oral device 1500 is disposed in the user’s mouth. In some embodiments, the pliable retention material can be formed on a first side (e.g., an upper side) of the curved frame 1510 such that the pliable retention material can engage the upper teeth of the user when the oral device is disposed in the user’s mouth. In some embodiments, in addition to or in the alternative to the pliable retention material being formed on the first side of the curved frame 1510, the pliable retention material can also be formed on a second side of the curved frame 1510 (e.g., a lower side) opposite the first side such that the pliable retention material can engage the lower teeth of the user when the oral device is disposed in the user’s mouth. In some embodiments, the oral device 1500 can be symmetrical about a plane extending through the curved frame such that the first side of the curved frame can engage either the upper or lower teeth of the user and the second side of the curved frame can engage either the lower or upper teeth of the user, respectively, when the oral device 1500 is disposed in the mouth of the user. In use, the oral device 1500 can allow the user to swallow (e.g., swallow saliva) when the oral device 1500 is disposed within the user’s mouth.

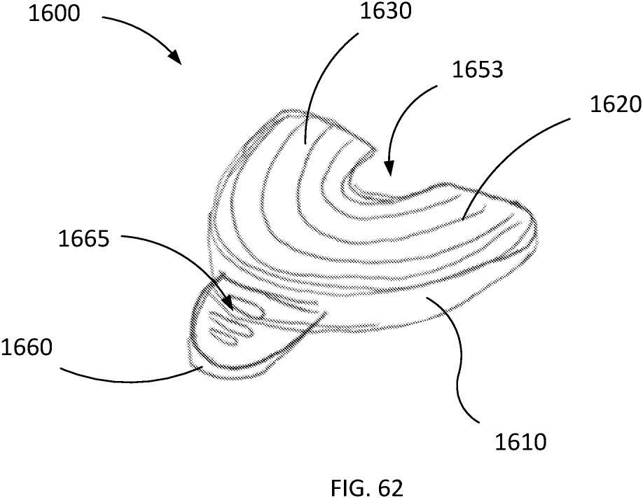

FIG. 62 is a front perspective view of an oral device 1600. The oral device 1600 can be the same or similar in structure and/or function to any of the oral devices described herein, such as the oral device 100 and/or the oral device 300. For example, the oral device 1500 includes a curved frame 1610 and a curved bite block 1601. In some embodiments, the curved frame 1610 and the curved bite block 1601 can be shaped as a wedge. The curved bite block 1601 includes a first bite block portion 1620 and second bite block portion 1630. In some embodiments, the oral device 1600 includes a handle 1660 which can include handle grip portions 1665 (e.g., ridges or bumps). In some embodiments, the oral device 1600 defines a notch 1653 (e.g., a curved notch) that can be configured to receive a portion of the user’s tongue such that the tip of the user’s tongue can be retained on a first side of the oral device 1600 (e.g., within the user’s lower jaw) but a proximal portion of the user’s tongue can be disposed within the notch 1653. In some embodiments, the curved bite block 1601 can include contoured regions that in use will aid the placement of the tongue of the user when the device 1600 is disposed in the user’s mouth. In some embodiments, portions of the curved frame 1610, first bite block 1620, and/or second bite block 1630 form an arc that corresponds to the shape of the user’s upper and/or lower jawline. In some embodiments, the device 1600 forms an arc having a lateral extent (e.g., a width at the widest portion of the device 1600) that is wider than the outer perimeter of the user’s teeth such that device 1600 applies pressure to the user’s buccal tissues (e.g., inner cheeks) to urge the buccal tissues away from the teeth biting surfaces. In some embodiments, the notch 1653 is sufficiently small (e.g., in diameter and/or width) such that a first or second surface of the device 1600 (e.g., a first or second surface of the bite block 1601) can extend inwardly relative to a user’s teeth and can depress the superior surface of the user’s tongue towards the user’s lower jaw and away from the user’s biting plane. In use, the oral device 1600 can allow the user to swallow (e.g., swallow saliva) when the oral device 1600 is disposed within the user’s mouth.

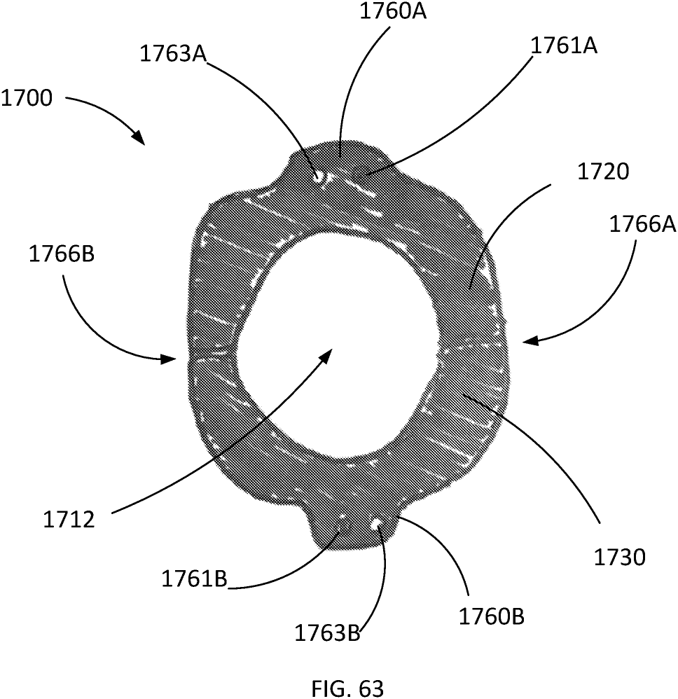

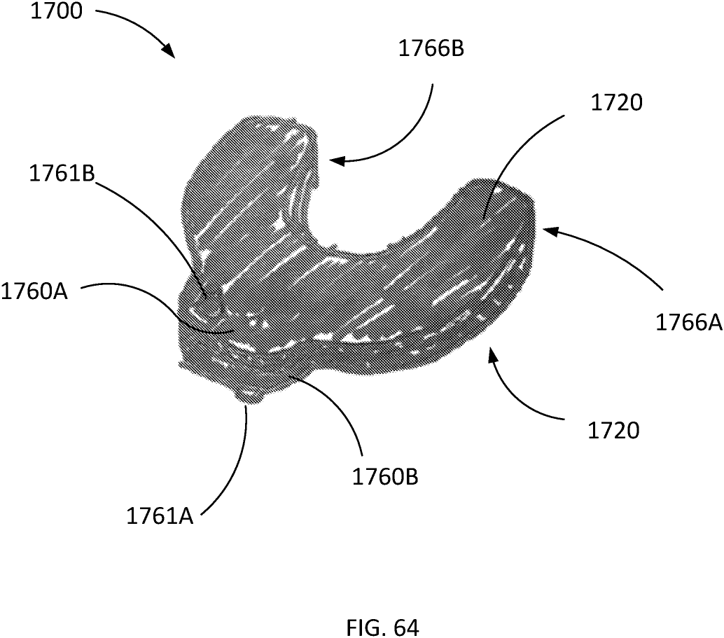

FIGS. 63 and 64 are a front open view and a front perspective closed view, respectively, of an oral device 1700. The oral device 1700 can be the same or similar in structure and/or function to any of the oral devices described herein, such as the oral device 100 and/or the oral device 300. The oral device 1700 has an open configuration, as shown in FIG. 63, and a closed configuration, as shown in FIG. 64. The oral device 1700 includes an upper bite block portion 1720 having an upper biting surface, a lower bite block portion 1730 having a lower biting surface, and an opening 1712. In some embodiments, the oral device 1700 includes an upper handle 1760A (also referred to as an upper tab) a lower handle 1760B (also referred to as a lower tab). The oral device 1700 includes a first hinge 1766A and a second hinge 1766B. Each of the first hinge 1766A and the second hinge 1766B can be a living hinge. The first hinge 1766A and second hinge 1766B can be configured such that, when the oral device 1700 is disposed within the user’s mouth, the oral device 1700 can open and close with the user’s mouth (i.e., the first hinge 1766A and second hinge 1766B allows the oral device 1700 to assume a closed configuration and an open configuration). In some embodiments, the first hinge 1766A and second hinge 1766B optionally include sufficient stiffness and/or spring-like mechanisms to assist the oral device 1700 to return to an open configuration (e.g., the first hinge 1766A and the second hinge 1766B can be biased toward the open configuration). In use, the oral device 1700 can prevent accidental dislodging of the oral device 1700 due to the securement of the oral device 1700 to the teeth and mouth via the first hinge 1766A and second hinge 1766B. In use, the first hinge 1766A and second hinge 1766B allow the user to close their lips when the oral device 1700 is disposed within the user’s mouth, increasing comfort and decreasing excessive salivation. The oral device 1700 can be stored in the open configuration or the closed configuration. In some embodiments, the central opening of the oral device 1700 can be secured to a package (e.g., via a friction fit with a projection of the package) for storage. In some embodiments, the oral device 1700 can be transitioned from the open configuration shown in FIG. 63 to the closed configuration shown in FIG. 62 by the user or caregiver folding the upper bite block portion 1720 toward the lower bite block portion about the first hinge 1766A and the second hinge 1766B. At least one of the upper handle 1760A and the lower handle 1760B includes a post having a snap feature (e.g., an outwardly extending flange disposed on the end of the post) configured to be pushed through an opening of and secured to the other of the upper handle 1760A or the lower handle 1760B. For example, the upper handle 1760A includes a first post 1761A and defines a first opening 1763A. The lower handle 1760B includes a second post 1761B and defines a second opening 1763B. The first post 1761A can be engaged with and secured to the lower handle 1760B via the second opening 1763B and the second post 1761B can be engaged with and secured to the upper handle 1760A via the first opening 1763A. In use, the oral device 1700 can allow the user to swallow (e.g., swallow saliva) when the oral device 1700 is disposed within the user’s mouth.

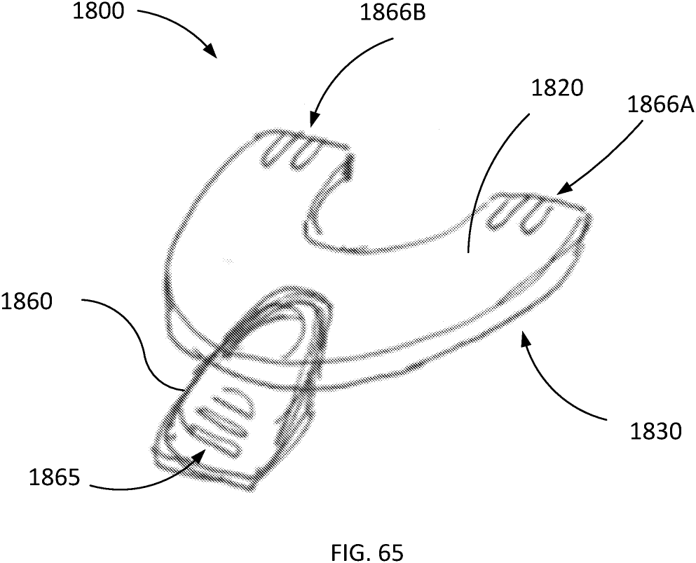

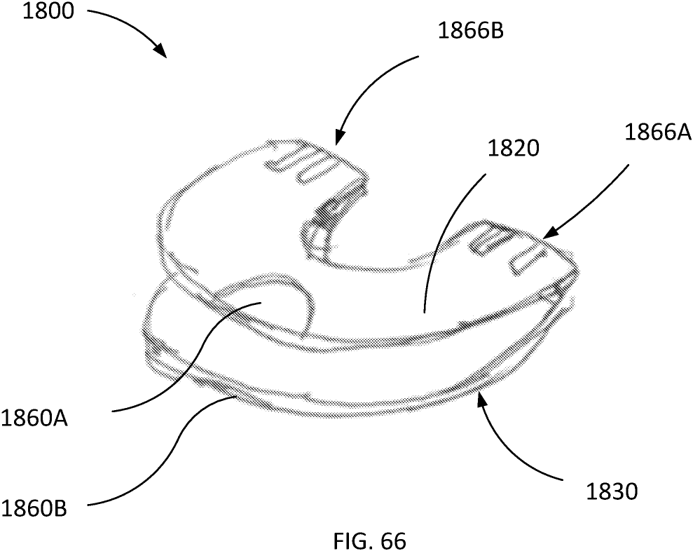

FIGS. 65 and 66 are front perspective views of an oral device 1800. The oral device 1800 can be the same or similar in structure and/or function to any of the oral devices described herein, such as the oral device 100 and/or the oral device 1700. The oral device 1800 can assume a closed configuration (as in FIG. 65) or an open configuration (as in FIG. 66). The oral device 1800 includes an upper bite block portion 1820 having an upper biting surface and a lower bite block portion 1830 having a lower biting surface. In some embodiments, the oral device 1800 includes one or more handles 1860, which can include handle grip portions 1865 (e.g., ridges or textured areas). The handle 1860 can be configured to be detached from the oral device 1800 (e.g., via separating a thinned or perforated region between the handle 1860 and the upper bite block portion 1820 and/or the lower bite block portion 1830). The oral device 1800 includes a first hinge 1866A and a second hinge 1866B. Each of the first hinge 1866A and the second hinge 1866B can be a living hinge. The first hinge 1866A and second hinge 1866B can be configured such that, when the oral device 1800 is disposed within the user’s mouth, the oral device 1800 can open and close with the user’s mouth (i.e., the first hinge 1866A and second hinge 1866B allow the oral device 1800 to assume a closed configuration and an open configuration). In some embodiments, the first hinge 1866A and second hinge 1866B optionally include sufficient stiffness and/or spring-like mechanisms to assist the oral device 1800 to return to an open configuration (e.g., the first hinge 1866A and the second hinge 1866B can be biased toward an open configuration).

In some embodiments, the handle 1860 maintains the oral device 1800 in the closed configuration by maintaining the upper bite block portion 1820 against, parallel to, or near the lower bite block portion 1830 such that the oral device 1800 cannot transition between the closed configuration and the open configuration until the handle 1860 is detached from the upper bite block portion 1820 and the lower bite block portion 1830. In some embodiments, the handle 1860 can be detachable and re-attachable from a first handle receptor 1860A and a second handle receptor 1860B shown in FIG. 66. When the first hinge 1866A and the second hinge 1866B are open, as depicted in FIG. 66, the tongue of the user can be disposed in the semi-circular opening defined by the upper bite block portion 1820 and the lower bite block portion 1830 of the oral device 1800. In some embodiments, the oral device 1800 can be configured such that the tongue of the user is not constrained by the oral device 1800 when the oral device 1800 is disposed within the user’s mouth and in an open or closed configuration (e.g., the user can move the tongue within a space defined by the semi-circular opening and/or between the upper bite block portion 1820 and the lower bite block portion 1830). In some embodiments, a tongue retention portion can be disposed between the upper bite block portion 1820 and the lower bite block portion 1830 defining the tongue area to prevent the tongue from traveling between the upper bite block portion 1820 and the lower bite block portion 1830. The tongue retention portion can be formed of, for example, expanded polytetrafluoroethylene (ePTFE). In use, the oral device 1800 can prevent accidental dislodging of the oral device 1800 due to the securement of the oral device 1800 to the teeth and mouth via the first hinge 1866A and second hinge 1866B.

In use, the first hinge 1866A and second hinge 1866B allow the user to close their lips when the oral device 1800 is disposed within the user’s mouth, increasing comfort and decreasing excessive salivation. In use, the oral device 1800 can allow ejection of matter through the oral cavity (e.g., vomit) and the ingestion of matter through the oral cavity (e.g., food and drink) via the semi-circular opening defined by the upper bite block portion 1820 and the lower bite block portion 1830. In use, the oral device 1800 can allow the user to swallow (e.g., swallow saliva) when the oral device 1800 is disposed within the user’s mouth. In some embodiments, the handle(s) 1860 can assist the user or caregiver to insert the oral device 1800 into a user’s mouth and then subsequently detach from the handle receptors 1860A and 1860B to provide a minimal protrusion from the mouth, which can be preferable in situations such as sleeping. In some embodiments, the handle 1860 can be detachable and re-attachable from a first handle receptor 1860A and a second handle receptor 1860B shown in FIG. 66. To remove the oral device 1800 from the user’s mouth, the user or caregiver can first re-attach the handle(s) 1860 to the oral device 1800 via the handle receptors 1860A and 1860B. In use, the handle(s) 1860 allow the oral device 1800 to be administered to the user without inserting hands and/or fingers into the mouth of the user, which can prevent injury during situations such as during a seizure. In use, the oral device 1800 can be administered to the mouth of the user by the user or caregiver before onset of a seizure (e.g., before the user’s jaws clamp together, thus increasing the difficulty with which to insert the oral device 1800 into the mouth of the user). In use, the oral device 1800 can be administered to the mouth of the user by the user or caregiver during an aura signaling an upcoming onset of a seizure.

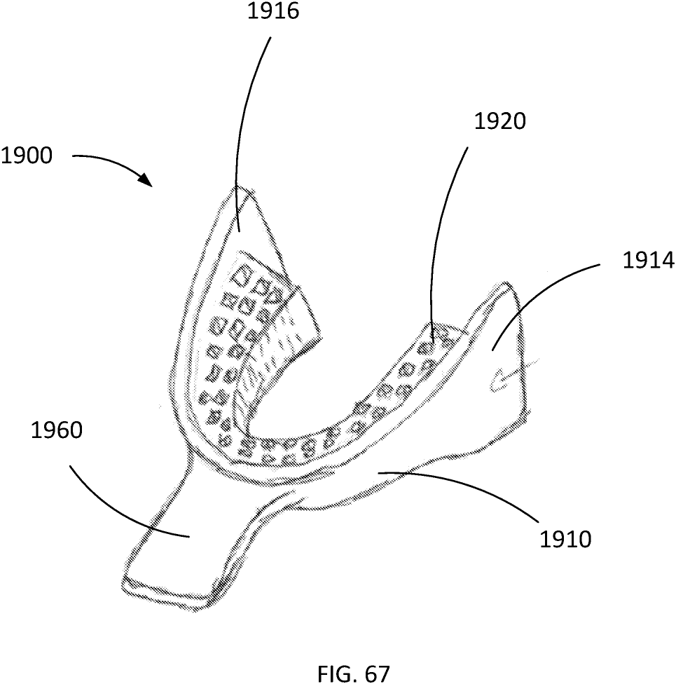

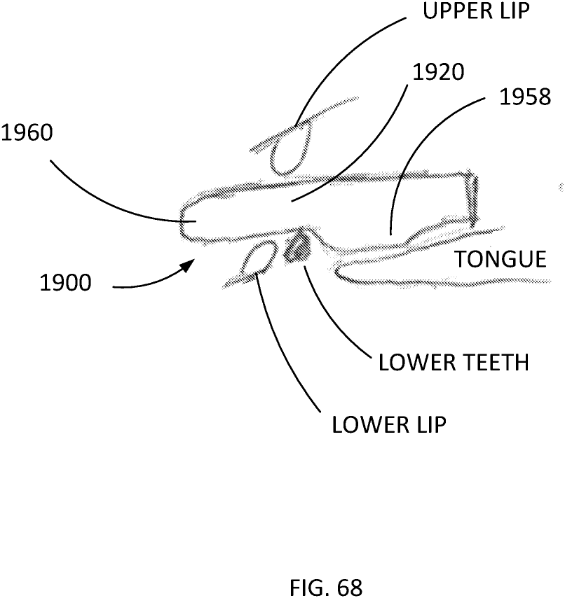

FIGS. 67 and 68 are front perspective and side views of an oral device 1900. The oral device 1900 can be the same or similar in structure and/or function to any of the oral devices described herein, such as the oral device 100 and/or the oral device 700. The oral device 1900 includes a curved frame 1910, a curved bite block 1920, a first cheek protector 1914, and a second cheek protector 1916. As shown in FIG. 67, in some embodiments, the curved frame 1910 can include or be formed monolithically with the first cheek protector 1914 and the second cheek protector 1916. The curved bite block 1920 can include an upper biting surface and a lower biting surface. In some embodiments, the oral device 1900 includes a low profile handle 1960. In some embodiments, a concave area defined by the curved bite block 1920 is empty to allow for tongue movement within the concave area when then oral device 1900 is disposed within the mouth of a user. In some embodiments (as shown in FIG. 68), the bite block 1920 includes a tongue depressor 1958 which can apply pressure on the superior surface of the user’s tongue (i.e., towards the lower jaw) when the oral device 1900 is disposed within the mouth of a user. In some embodiments, a portion of the tongue depressor 1958 can be tapered to guide the user’s tongue under the tongue depressor 1958 when the oral device 1900 is being inserted into the mouth of the user. In some embodiments, the tongue depressor 1958 occupies only a portion of the concave area within the curved bite block 1920, such that when the oral device 1900 is inserted into the mouth of the user, the tongue depressor 1958 does not inadvertently apply pressure on the user’s tongue in a direction other than towards the user’s lower jaw (e.g., a proximal pressure to urge the user’s tongue towards the user’s throat). In some embodiments, the tongue depressor 1958 includes a fabric woven of any suitable material (e.g., nylon).

In use, the oral device 1900 can prevent accidental dislodging of the oral device 1900 due to the securement of the oral device 1900 to the teeth and mouth via the curved bite block 1920, and/or the tongue depressor 1958 within the concave area of the curved bite block 1920. In some embodiments, the handle 1960 can be a flexible tab that can protrude out of the user’s mouth when the oral device 1900 is disposed within the user’s mouth. In use, the handle 1960 allows the oral device 1900 to be administered to the user without inserting hands and/or fingers into the mouth of the user, which can prevent injury during situations such as seizure. In some embodiments, the handle 1960 can protrude from the user’s mouth no more than a distance sufficient to grasp the handle 1960 with fingertips, which may prevent the user or caregiver from inserting the oral device 1900 too far into the user’s mouth, and can also reduce the risk of inadvertent, external forces being applied to the handle 1960 (e.g., during seizure) that might force the oral device 1900 too far into the user’s mouth. In some embodiments, the handle 1960 can be configured to detach from the oral device 1900, which can reduce the risk of inadvertent, external forces being applied to the oral device 1900 that may disrupt function and/or position of the oral device 1900 when the oral device 1900 is disposed within the mouth of the user. For example, a user or caregiver can use the handle 1960 to insert the oral device 1900 into the user’s mouth and then detach the handle 1960 from the oral device 1900 before the user inadvertently moves their mouth to make contact with an external object (e.g., a pillow during sleep). In use, the handle 1960 and/or curved frame 1910 of the oral device 1900 can keep the user’s lips from contacting each other when the oral device 1900 is disposed within the user’s mouth (as shown in FIG. 68), which may aid in natural drainage of saliva and/or inserting matter into the user’s mouth (e.g., a catheter and/or medication). In some embodiments, the oral device 1900 can include an array of openings or apertures throughout the curved bite block 1920. In some embodiments, the array of openings or apertures throughout the curved bite block 1920 extend vertically through the curved bite block 1920 (i.e., from the lower jaw surface of the curved bite block 1920 to the upper jaw surface of the curved bite block 1920). The curved bite block 1920 can be a sufficient stiffness such that when the oral device 1900 is disposed within the mouth of the user, the user can bite down on the curved bite block 1920 and the curved bite block 1920 will buckle against the bite force without tearing and then expand back to the original volume of the bite block 1920 when bite force is reduced or removed. In use, the array of openings or apertures throughout the curved bite block 1920 allow for fluids to pass through the oral device 1900 (e.g., for drainage). In use, the oral device 1900 can allow the user to swallow (e.g., swallow saliva) when the oral device 1900 is disposed within the user’s mouth.

In some embodiments, the first cheek protector 1914 and second cheek protector 1016 can be disposed on the curved frame 1910 such that when the oral device 1900 is disposed within the mouth of the user, the first cheek protector 1914 and second cheek protector 1916 can separate the teeth engaged with the curved bite block 1920 from the user’s cheeks. The first cheek protector 1914 and second cheek protector 1916 can be made of any material with sufficient stiffness such that the first cheek protector 1914 and second cheek protector 1916 can be temporarily deformed during insertion of the oral device 1900 into the mouth of the user. In some embodiments, the first cheek protector 1914 and second cheek protector 1916 extend from the curved frame 1910 a sufficient distance such that when the oral device 1900 is disposed in the mouth of the user, the first cheek protector 1914 and second cheek protector 1916 prevent the oral device 1900 from translating along the upper or lower jaws of the user. The curved bite block 1920 can be configured to engage some or all of the teeth in the user’s upper jaw and/or lower jaw. In some embodiments, the curved bite block 1920 is made of one material. In some embodiments, the curved bite block 1920 is made of an inner material and an outer material (e.g., coating). The inner material can have an increased stiffness relative to the outer material.

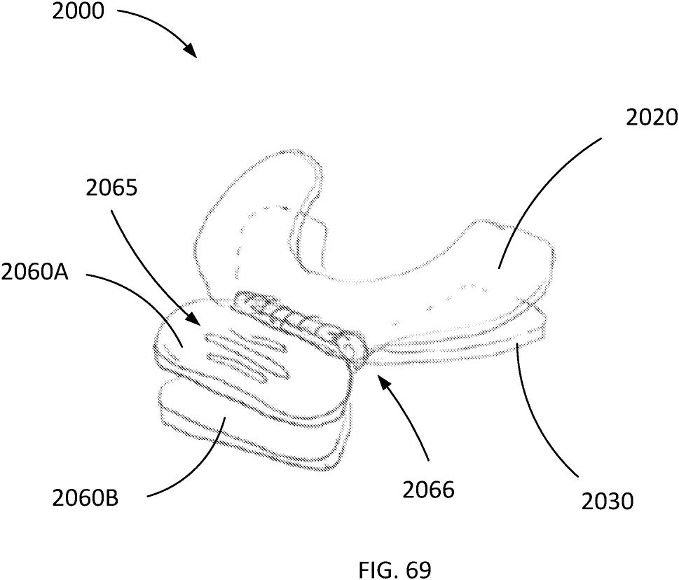

FIG. 69 is a front perspective view of an oral device 2000. The oral device 2000 can be the same or similar in structure and/or function to any of the oral devices described herein, such as the oral device 100 and/or the oral device 1700. The oral device 2000 can assume an open configuration or a closed configuration. The oral device 2000 includes a curved upper biting block 2020 having an upper biting surface and a curved lower biting block 2030 having a lower biting surface. In some embodiments, the upper biting block 2020 is formed to engage the surface of all or a portion of the teeth in the user’s upper jaw. In some embodiments, the lower biting block 2030 is formed to engage the surface of all or a portion of the teeth in the user’s lower jaw. In some embodiments, the upper biting block 2020 and/or lower biting block 2030 can define a concave area that allows for tongue movement within the concave area when the oral device 200 is disposed within the mouth of the user. In some embodiments, the upper biting block 2020 and/or lower biting block 2030 can define a concave area that is non-empty and includes a tongue depressor which can apply pressure on the superior surface of the user’s tongue (i.e., towards the lower jaw) when the oral device 2000 is disposed within the user’s mouth. In some embodiments, the upper biting block 2020 and/or lower biting block 2030 can form a concave area that includes a lingual frame which can engage any or all surfaces of the tongue to constrict motion of the user’s tongue when the oral device 2000 is disposed within the user’s mouth (e.g., to prevent the tongue from being disposed between the upper biting block 2020 and the lower biting block 2030. In some embodiments, the upper biting surface 2020 and/or lower biting surface 2030 is angled and/or tapered to accommodate the jaw angle of the user when the oral device 2000 is disposed within the user’s mouth.

In some embodiments, the oral device 2000 includes an upper handle 2060A and a lower handle 2060B, which can include suitable grip portions 2065 (e.g., ridges). The oral device 2000 includes a hinge 2066. The hinge 2066 can be configured such that, when the oral device 2000 is disposed within the user’s mouth, the oral device 2000 can be transitioned between a closed configuration and an open configuration. In some embodiments, the hinge 2066 includes sufficient stiffness and/or spring-like mechanisms to assist the oral device 2000 to return to an open configuration (e.g., the hinge 266 is biased toward an open configuration).

In use, the oral device 2000 can prevent accidental dislodging of the oral device 2000 due to the securement of the oral device 2000 to the teeth and mouth via the hinge 2066. In use, the hinge 2066 allows the user to close their lips when the oral device 2000 is disposed within the user’s mouth, increasing comfort and decreasing excessive salivation. In some embodiments, the hinge is biased toward the closed configuration but can be maintained in an open configuration by a ratcheting mechanism coupled to or disposed within the hinge that can be selectively locked in one or more expanded configurations. In some embodiments, the hinge 2066 is disposed between the handle (e.g., upper handle 2060A and lower handle 2060B) and biting blocks (e.g., upper biting block 2020 and lower biting block 2030) such that motion of the upper handle 2060A towards the lower handle 2060 (e.g., squeezed together) corresponds to motion of the upper biting surface 2020 away from the lower biting surface 2030. In use, the user and/or caregiver can insert the oral device 2000 in a closed configuration into the user’s mouth via the handle. The user and/or caregiver can then squeeze the upper handle 2060A and lower handle 2060B together to create an open configuration of the oral device 2000 such that the distance between the upper biting surface 2020 and lower biting surface 2030 is at a preferred distance of the user and/or caregiver (e.g., to custom fit the open configuration to the size of the user’s oral cavity). The user and/or caregiver can then release the upper handle 2060A and lower handle 2060B and the oral device 2000 will maintain the prescribed open configuration. In some embodiments, the oral device 2000 is vertically symmetrical such that either side can be placed in contact with the upper or lower teeth. In use, the oral device 2000 can allow the user to swallow (e.g., swallow saliva) when the oral device 2000 is disposed within the user’s mouth.

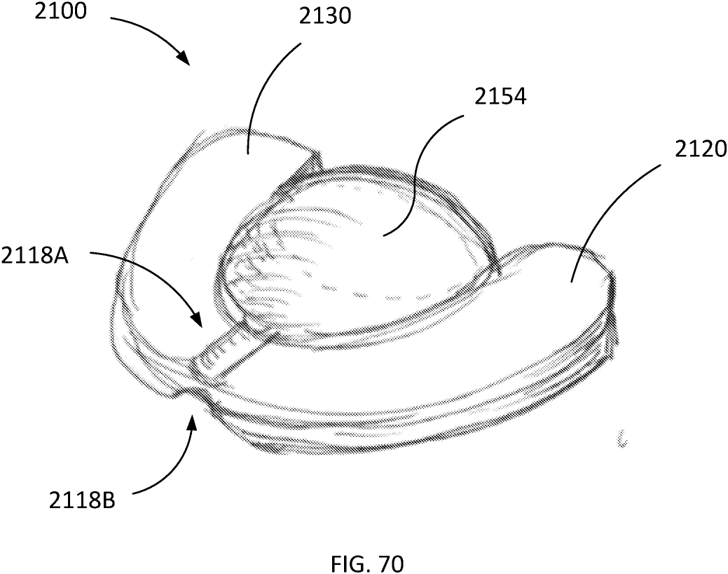

FIG. 70 is a front perspective view of an oral device 2100. The oral device 2100 can be the same or similar in structure and/or function to any of the oral devices described herein, such as the oral device 100 and/or the oral device 300. The oral device 2100 includes a first bite block 2120 and a second bite block 2130. In some embodiments, the oral device 2100 includes an upper notch 2118A and a lower notch 2118B, which can create a flexible axis dividing the first bite block 2120 and second bite block 2130. Although not shown, in some embodiments, the oral device 2100 includes a handle. The oral device 2100 includes a lingual frame 2154. In some embodiments, the lingual frame 2154 can include an upper portion and a lower portion defining a cavity to receive the tongue. In some embodiments, the lingual frame 2154 can define a boundary of a cavity to receive the tongue collectively with the floor of the user’s mouth. The lingual frame 2154 can be configured such that the oral device 2100 will not be ejected from the oral cavity when the oral device 2100 is disposed within the mouth of a user and the user engages the surface of the lingual frame 2154 with their tongue (e.g., during tongue convulsions and thrusts). For example, the lingual frame 2154 can be made of any suitable material and/or formed to any suitable shape such that the lingual frame 2154 can absorb contact forces from the tongue during tongue convulsions. In some embodiments, the lingual frame can apply pressure to the superior side of the user’s tongue (i.e., towards the lower jaw) when the oral device 2100 is disposed within the user’s mouth. In use, the oral device 2100 increases comfort in the user’s mouth when the oral device 2100 is disposed within the user’s mouth via the axis of flexibility between notches 2118A and 2118B, which decouples the stiffness between the first bite block 2120 and second bite block 2130 (e.g., forming a hinge portion between the first bite block 2120 and the second bite block 2130). In some embodiments, the upper notch 2118A can be used as a channel for the flow of air, saliva, and/or medication between the interior of the lingual frame 2154 and outside of the mouth. In some embodiments, the oral device 2100 is vertically symmetrical such that either side can be placed in contact with the upper or lower teeth. In some embodiments, portions of the first bite block 2120, and/or second bite block 2130 form an arc wider than the shape of the ridge of teeth forming the upper jaw and/or lower jaw such that portions of the first bite block 2120, and/or second bite block 2130 apply pressure to the buccal tissues (e.g., inner cheeks) to urge the buccal tissues away from the teeth biting surfaces between the upper and lower jaws. In use, the oral device 2100 can allow the user to swallow (e.g., swallow saliva) when the oral device 2100 is disposed within the user’s mouth.

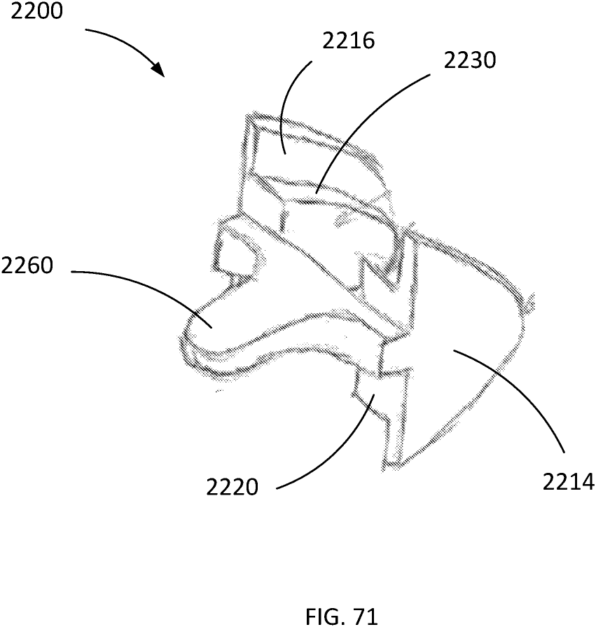

FIG. 71 is a front perspective view of an oral device 2200. The oral device 2200 can be the same or similar in structure and/or function to any of the oral devices described herein, such as the oral device 100 and/or the oral device 300. The oral device 2200 includes a first bite block 2220, a first cheek protector 2214, a second bite block 2230, and a second cheek protector 2216. In some embodiments, the oral device 2200 includes a handle 2260. In some embodiments, the first bite block 2220 and second bite block 2230 can be configured to keep the user’s mouth open when the oral device 2200 is disposed in the mouth of the user. In use, the oral device 2200 can prevent excessive movement of the jaw that may occur during an episode of movement disorder (e.g., during seizure). In use, the oral device 2200 can force the user’s upper and lower jaw apart to reduce the user’s potential biting force (e.g., during an episode of uncontrolled biting such as during a seizure). In use, the oral device 2200 can allow the user to swallow (e.g., swallow saliva) when the oral device 2200 is disposed within the user’s mouth.

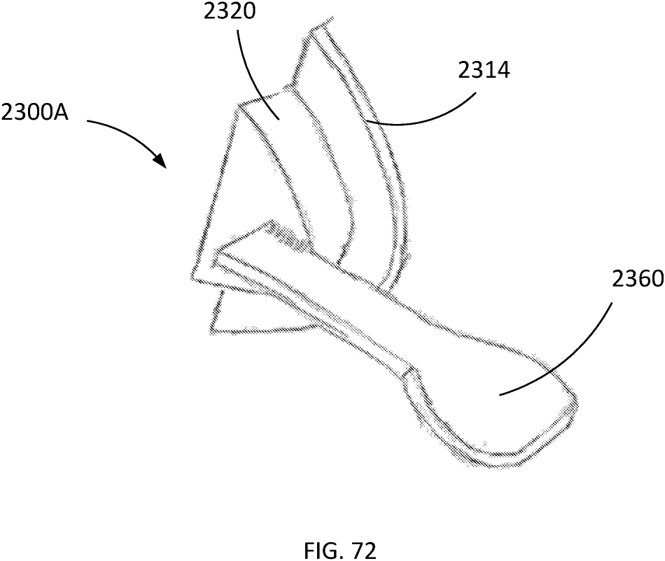

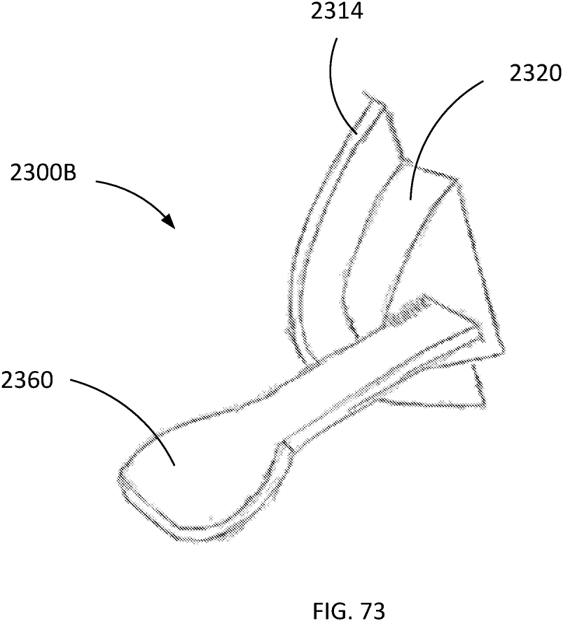

FIGS. 72 and 73 are front perspective views of a first oral device 2300A and a second oral device 2300B. The oral device 2300A and the oral device 2300B can be the same or similar in structure and/or function to any of the oral devices described herein, such as the oral device 100 and/or the oral device 2200. The oral device 2300A and the oral device 2300B each include a single bite block 2320 and a single cheek protector 2314. In some embodiments, the oral device 2300A and/or the oral device 2300B can include a handle 2360. Although shown as extending away from the proximal portion of the first oral device 2300A and the proximal portion of the second oral device 2300B, the handle 2360 can extend away from the distal portion of the first oral device 2300A and the distal portion of the second oral device 2300B, respectively. In some embodiments, the bite block 2320 and/or cheek protector 2314 can be angled and/or tapered to accommodate the jaw angle of the user when the oral device 2300A or oral device 2300B is disposed in the user’s mouth. In some implementations, the oral device 2300A can be the same embodiment of oral device 2300B. In some embodiments, the bite block 2320 can be configured to keep the user’s mouth open when the oral device 2300 is disposed in the mouth of the user. In use, the oral device 2300 can prevent excessive movement along one side of the mouth that may occur during an episode of movement disorder (e.g., during seizure). The oral device 2300A or oral device 2300B can be independently disposed within the user’s mouth to prevent the user from inadvertently biting their tongue and/or prevent the user from moving their jaw (i.e., the user’s jaw can be secured by using either oral device 2300A or oral device 2300B). When the first oral device 2300A and the second oral device 2300B are simultaneously disposed within the user’s mouth, the oral devices 2300A, 2300B can be disposed on opposite sides of the user’s mouth. Although not shown, in some embodiments the cheek protector 2314 can extend away from the bite block 2320 (e.g., a distance as wide or wider than the bite block 2320) to push the cheek away from the teeth. In use, the oral device 2300A and the oral device 2300B can allow the user to swallow (e.g., swallow saliva) when the oral device 2300A or the oral device 2300B is disposed within the user’s mouth.

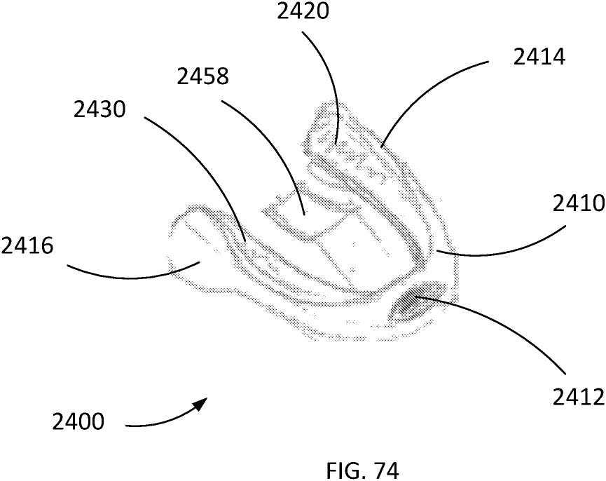

FIG. 74 is a front perspective view of an oral device 2400. The oral device 2400 can be the same or similar in structure and/or function to any of the oral devices described herein, such as the oral device 100 and/or the oral device 300. The oral device 2400 includes a curved frame 2410, a distal opening 2412, a first bite block 2420, a first cheek protector 2414, a second bite block 2430, and a second cheek protector 2416. In use, the distal opening 2412 can allow air and/or fluid to pass through the oral device 2400 when the oral device 2400 is disposed within the mouth of a user and the user is biting down on the first bite block 2420 and second bite block 2430. For example, the distal opening 2412 can allow for respiration (e.g., unobstructed breathing) and/or draining of oral fluids. As another example, the distal opening 2412 can allow oral fluids to drain out of the patient’s mouth (e.g., to reduce the risk of choking on oral fluids). In some embodiments, the oral device 2200 includes a tongue depressor 2458. The tongue depressor 2458 can be coupled to and extend proximally from the curved frame 2410. For example, the tongue depressor 2458 can be coupled to a portion of the curved frame 2410 below the distal opening 2412. The tongue depressor can include a cantilever biased downward (e.g., away from an upper biting surface and/or a lower biting surface of the oral device 2400). The tongue depressor 2458 can be, for example, semi-rigid or elastomeric. The tongue depressor 2458 can be configured such that, when the oral device 2400 is disposed within the user’s mouth, the tongue depressor 2458 can apply pressure on the superior surface of the user’s tongue to urge the user’s tongue towards the user’s lower jaw and away from the user’s bite plane. In use, the oral device 2400 can prevent accidental dislodging of the oral device 2400 due to the securement of the oral device 2400 to the mouth via the tongue depressor 2458. In some embodiments, the first bite block 2420 and the second bite block 2430 can be formed of one material or a first inner material coated with a second outer material that is softer than the inner material. The outer material can be depressable or otherwise deformable by the user’s teeth as a result of a bite force, while the inner material can be sufficiently rigid to not deform under the bite force. Although not shown, in some embodiments, the device 2400 can include a handle similar to or the same as any of the handles described herein (e.g., disposed below and/or above the distal opening 2412). In use, the oral device 2400 can allow the user to swallow (e.g., swallow saliva) when the oral device 2400 is disposed within the user’s mouth.

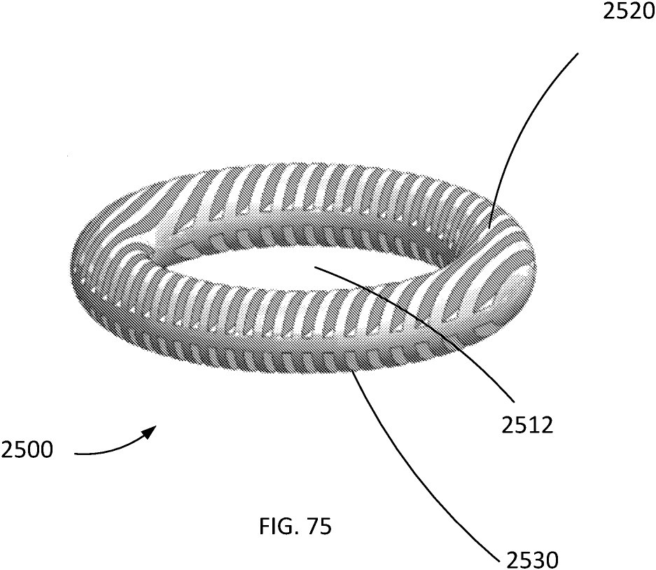

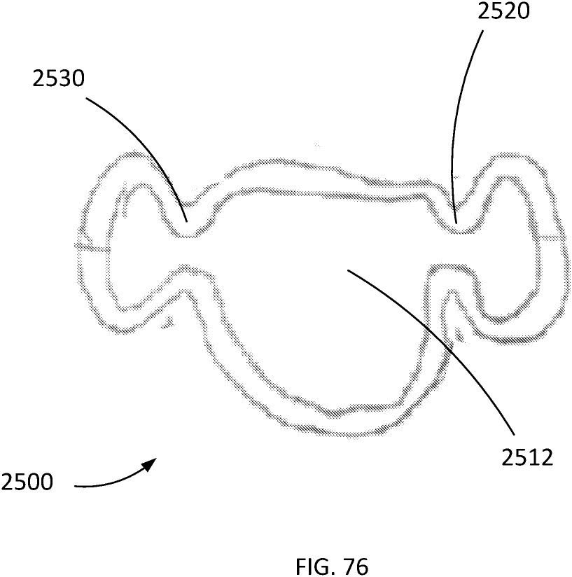

FIGS. 75 and 76 are front perspective views of an oral device 2500 in separate configurations. The oral device 2500 can be the same or similar in structure and/or function to any of the oral devices described herein, such as the oral device 100. The oral device 2500 includes an opening 2512, a first biting surface 2520, and a second biting surface 2530. In some embodiments, as shown in FIG. 75, the oral device 2500 is formed as a ring. In some embodiments, rather than being formed as a ring, the oral device 2500 can be formed as a concave cup, hollow cylinder, or any other suitable shape that defines an interior space or an opening 2512. The oral device 2500 is made of a compressive material that allows for the oral device 2500 to be compressed and deformed to a plurality of configurations in which the user’s teeth can contact a portion of the first biting surface 2520 and a portion of the second biting surface 2530. In some embodiments, as shown in FIG. 75, the oral device 2500 includes a plurality of channels throughout the oral device 2500 which can increase the capacity of the oral device 2500 to be deformed into the plurality of configurations in which the user’s teeth can compress a portion of the first bite block 2520 toward a portion of the second bite block 2530. Additionally, oral fluids can flow into the plurality of channels and be removed from the user’s mouth with the oral device 2500. In use, the oral device 2500 can be inserted into the mouth of a user in any orientation in which the user’s teeth can contact a portion of the first biting surface 2520 and a portion of the second biting surface 2530. For example, in a horizontal configuration (as shown in FIG. 75) or in a vertical configuration (as shown in FIG. 76). In use, the oral device 2500 can be easily inserted into the mouth of a user by a user or caregiver without consideration as to the orientation of the oral device 2500, which can be helpful when the oral device 2500 needs to be placed in the mouth quickly and when the mouth of the user assumes an unexpected configuration, such as during an episode of uncontrolled movement (e.g., seizures). In some configurations, portions of the oral device 2500 (e.g., portions disposed outside of the user’s teeth when the oral device 2500 is secured under compression between the user’s upper and lower teeth in use) can be disposed between the user’s teeth and buccal tissues (e.g., inner cheeks) such that the buccal tissues are urged away from the biting surfaces of the user’s teeth. In some configurations, portions of the oral device 2500 can be disposed within the user’s oral cavity to engage the superior surface of the user’s tongue, which can prevent the tongue from contacting the biting surfaces of the user’s teeth. In some embodiments, the user’s tongue can be disposed within the opening 2512 to prevent the tongue from contacting the biting surfaces of the user’s teeth. For example, if the oral device 2500 is shaped as a concave cup and disposed within the user’s mouth, the user’s tongue can be disposed within the cavity of the cup. In some embodiments, the user’s tongue can be depressed below the oral device 2500 toward the floor of the user’s mouth by the bottom surface of the oral device 2500 (e.g., when the oral device 2500 is shaped as a ring inserted horizontally as shown in FIG. 73). The oral device 2500 can be sufficiently large (e.g., have a sufficiently large diameter) such that a user is unable to swallow the oral device 2500. In some embodiments, the oral device 2500 is biased towards an uncompressed and undeformed configuration (e.g., biased towards the initial shape of the ring, concave cup, or hollow cylinder before insertion into the user’s mouth), which can urge the user’s mouth to remain open when the oral device 2500 is disposed in the user’s mouth. In use, the oral device 2500 can allow the user to swallow (e.g., swallow saliva) when the oral device 2500 is disposed within the user’s mouth.

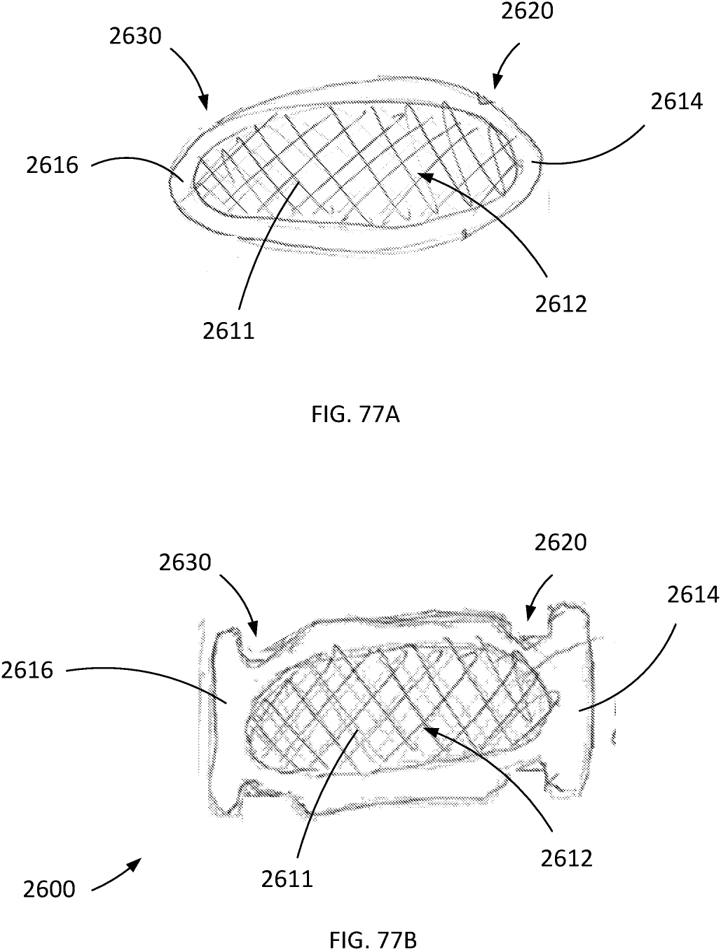

FIGS. 77A and 77B are a front views of an oral device 2600 in a first configuration and a second configuration, respectively. The oral device 2600 can be the same or similar in structure and/or function to any of the oral devices described herein, such as the oral device 100 and/or the oral device 2500. The oral device 2600 includes an opening 2612, a first bite block 2620, a first cheek protector 2614, a second bite block 2630, and a second cheek protector 2616. In some embodiments, the enclosure that defines the opening 2612 can be formed as a ring, concave cup, hollow cylinder, or any other suitable shape. In some embodiments, the enclosure that defines the opening 2612 can include the first bite block 2620, the second bite block 2630, first cheek protector 2614, and/or second cheek protector 2616. The oral device 2600 is made of a material that is able to be compressed and deformed to a plurality of configurations in which the user’s teeth can contact a portion of the first bite block 2620 and a portion of the second bite block 2630. For example, the oral device 2600 can be transitioned from the undeformed first configuration, as shown in FIG. 77A, to the deformed second configuration, as shown in FIG. 77B, when placed in the user’s mouth and compressed by the user’s teeth. The first configuration, for example, can have an ovular or circular perimeter when viewed from the front as shown in FIG. 77A. In some embodiments, the opening 2612 within the oral device 2600 contains a filter 2611 that allows air and/or fluid to pass through unobstructed but prevents foreign objects from entering the user’s throat. The filter 2611 can be disposed within all or a portion of the opening 2612. In some embodiments, the filter 2611 within the opening 2612 is an elastomeric mesh or grid. In use, the opening 2612 of the oral device 2600 can promote breathing and the expelling of excessive saliva and/or vomit (e.g., during a seizure or uncontrolled movement of the mouth) by allowing the passing of fluid and the filter 2611 can protect the user from foreign objects that could enter the user’s throat. In some embodiments, the oral device 2600 is biased towards an uncompressed and undeformed configuration (e.g., biased towards the initial shape of the oral device 2600 before insertion into the user’s mouth), as shown in FIG. 77A, which can urge the user’s mouth to remain open when the oral device 2600 is disposed in the user’s mouth and the user is not exerting a sufficient bite force to fully deform the oral device 2600 into the second configuration. In some configurations, portions of the oral device 2600 can be disposed within the user’s oral cavity to engage the superior surface of the user’s tongue, which can prevent the tongue from contacting the biting surfaces of the user’s teeth. In use, the oral device 2600 can allow the user to swallow (e.g., swallow saliva) when the oral device 2600 is disposed within the user’s mouth.

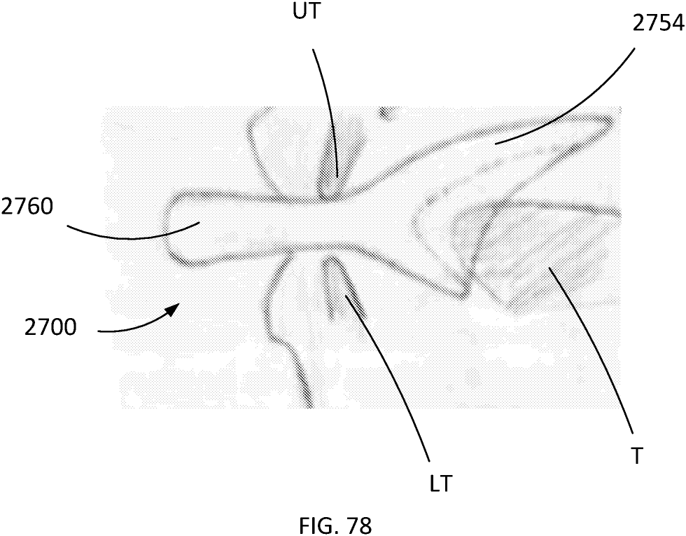

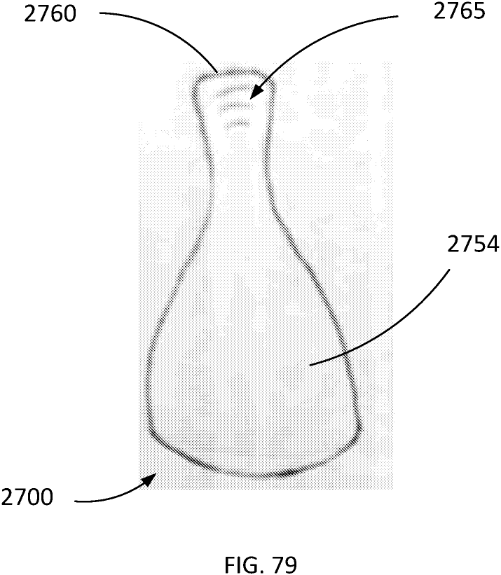

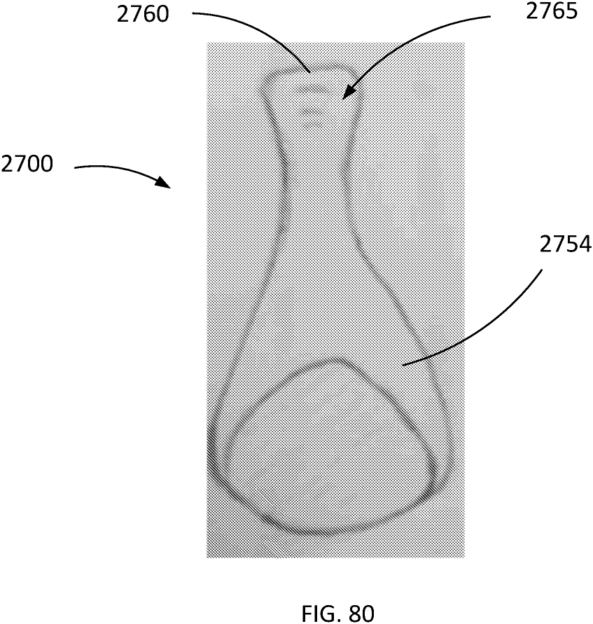

FIGS. 78-80 are the side, top, and bottom views, respectively, of an oral device 2700. FIG. 78 shows the oral device 2600 disposed relative to an upper tooth UT, a lower tooth LT, and a tongue T of a user. The oral device 2600 can be the same or similar in structure and/or function to any of the oral devices described herein, such as the oral device 100 and/or oral device 400. The oral device 2700 includes a lingual frame 2754 that can be the same or similar in structure and/or function to the concave cup described above with respect to oral device 2600. The oral device 2700 includes a handle 2760 to facilitate insertion, positioning, and removal of the oral device 2700, which can include a plurality of grip portions 2765 (e.g., raised portions or ridges) disposed along the handle 2760. In use, the lingual frame 2754 can encapsulate a distal portion of the tongue to restrict movement of the tongue (i.e., prevent the tongue from moving into the bite plane between the upper teeth and lower teeth) during seizures or episodes of uncontrolled jaw movement. As shown in FIG. 78, the lingual frame 2754 can have a cup-like shape defining a concave receiving area for the tongue. A lower portion of the lingual frame 2754 can extend a shorter distance proximally from the handle 2760 than an opposing upper portion of the lingual frame 2754 such that the user’s tongue is not uncomfortably pushed proximally (e.g., in the event that the distal end of the tongue does not seat within the concave receiving area during insertion of the oral device 2600 into the user’s mouth). The handle 2760 can be configured to be contacted by the upper and lower teeth of the user and to maintain the upper teeth spaced from the lower teeth. The lingual frame 2754 can be configured such that the oral device 2700 will not be ejected from the oral cavity when the oral device 2700 is disposed within the mouth of a user and the user engages the surface of the lingual frame 2754 with their tongue (e.g., during tongue convulsions and thrusts). For example, the lingual frame 2754 can be made of a deformable or compressible material such that the oral device 2700 is not dislodged by the user’s tongue during, for example, tongue convulsions. In use, the oral device 2700 can allow the user to swallow (e.g., swallow saliva) when the oral device 2700 is disposed within the user’s mouth.

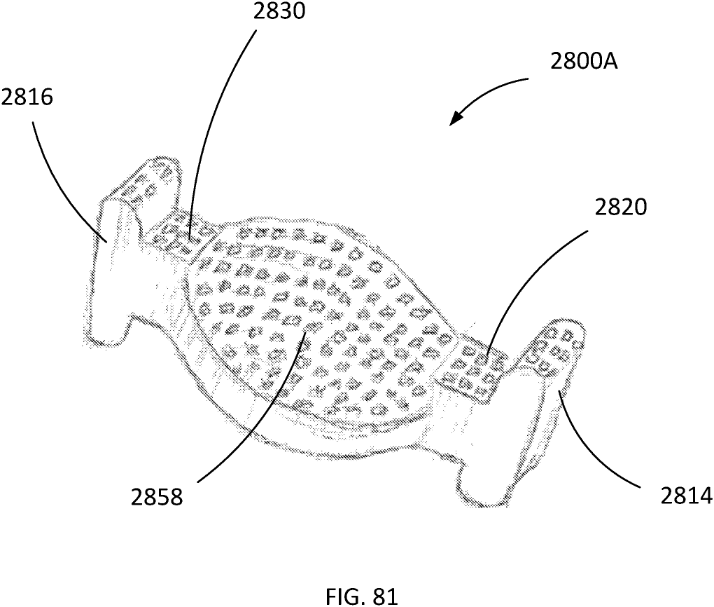

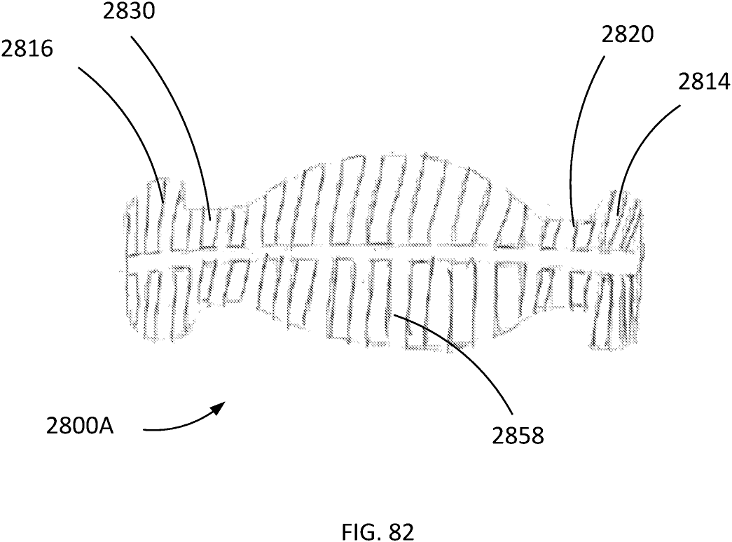

FIGS. 81-82 are a front perspective and a front cross-sectional view, respectively, of an oral device 2800A. The oral device 2800A can be the same or similar in structure and/or function to any of the oral devices described herein, such as the oral device 100. The oral device 2800A includes a first cheek protector 2814 coupled to a first bite block 2820 and a second cheek protector 2816 coupled to a second bite block 2830. The first bite block 2820 and second bite block 2830 are coupled to a tongue depressor 2858, which can be a sufficient thickness as to apply pressure on the superior surface of the user’s tongue when the oral device 2800A is disposed in the mouth of a user. The tongue depressor 2858 can be configured to apply pressure to the superior surface of the user’s tongue such that the user’s tongue is disposed at least partially within the user’s lower jaw and below the biting surfaces of the user’s teeth. In some embodiments, the tongue depressor 2858 can have a rounded or bulbous shape such that at least one of the upper surface and the lower surface are convex. In some embodiments, the oral device 2800A can include an array of openings or apertures throughout the oral device 2800A, such that fluid can pass between two sides of the oral device 2800A. In use, the array of openings or apertures through the oral device 2800A allows fluid (e.g., from excessive salivation) to easily travel within the mouth, which can help the user expel excessive fluids from the mouth. In some embodiments, the first cheek protector 2814 can extend away from the first bite block 2820 to push the user’s buccal tissues (e.g., inner cheeks) away from the teeth. In some embodiments, the second cheek protector 2816 can extend away from the second bite block 2830 to push the user’s buccal tissues (e.g., inner cheeks) away from the teeth. In some embodiments, the oral device 2800A can be configured to engage only a user’s tongue and proximal teeth (i.e., molars and/or premolars) such that when the oral device 2800A is disposed within the mouth of the user, the oral device does not obstruct the front of the user’s mouth. In some embodiments, the oral device 2800A can be formed of a material that can elastically deform under compressive forces (e.g., a user’s bite force) such that the material will not plastically deform and/or fracture and the material will resume its undeformed shape when the compressive forces are removed. In use, the material can allow the oral device 2800A to maintain separation between a user’s upper teeth and lower teeth when the oral device 2800A is disposed in the mouth of the user and the user is exerting a biting force upon the oral device 2800A.

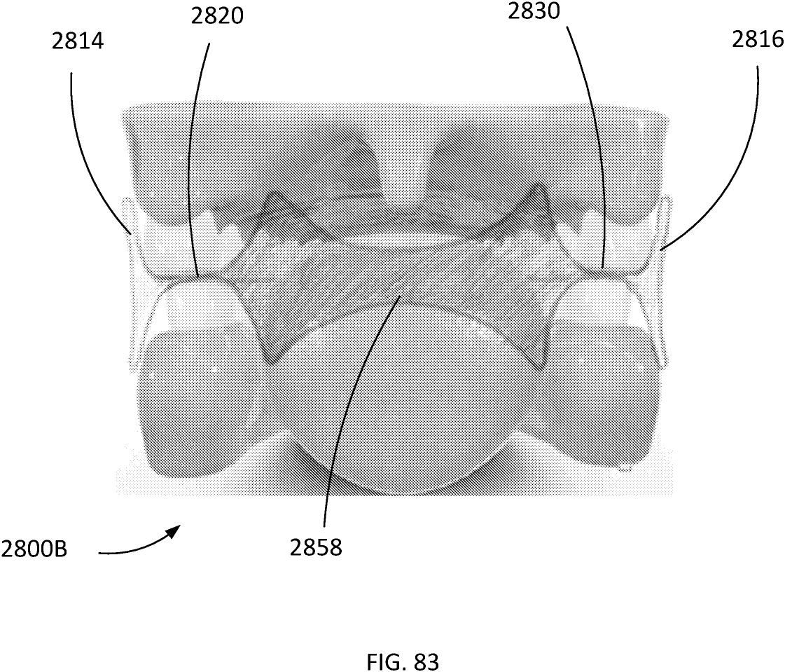

In some embodiments, rather than an oral device including a tongue depressor having an upper and lower convex surface, the upper and/or lower surface of the tongue depressor can be concave. For example, FIG. 83 is a posterior schematic view of an oral device 2800B. The oral device 2800B is an alternative embodiment of the oral device 2800A and thus can be the same or similar in structure and/or function to the oral device 2800A and/or any of the oral device described herein. For example, the oral device 2800B includes a first cheek protector 2814 coupled to a first bite block 2820 and a second cheek protector 2816 coupled to a second bite block 2830. The first bite block 2820 and second bite block 2830 are coupled to a tongue depressor 2858, which can be a sufficient thickness as to apply pressure on the superior surface of the user’s tongue when the oral device 2800B is disposed in the mouth of a user. The tongue depressor 2858 can be configured to apply pressure to the superior surface of the user’s tongue such that the user’s tongue is disposed in the user’s lower jaw and below the biting surfaces of the user’s teeth. As shown in FIG. 83, the tongue depressor 2858 includes an upper concave surface and a lower concave surface. Thus, in some embodiments, the upper and/or lower surfaces of the tongue depressor 2858 can define a tongue surface receiving area. In use, the oral device 2800A and the oral device 2800B can allow the user to swallow (e.g., swallow saliva) when the oral device 2800A or the oral device 2800B is disposed within the user’s mouth.

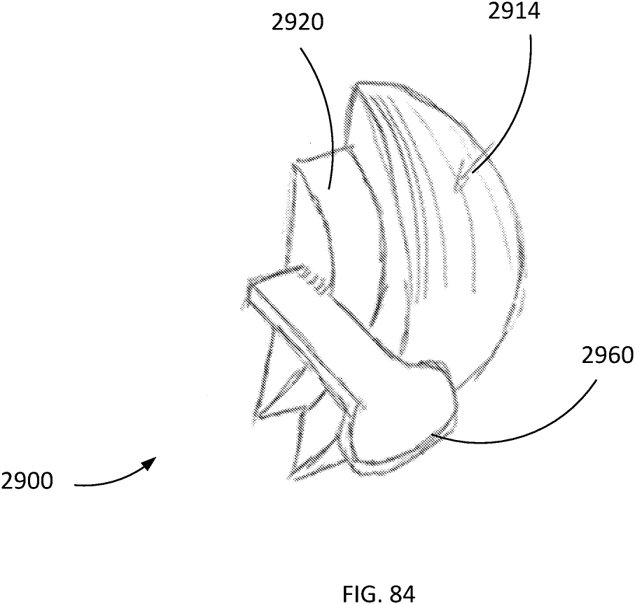

FIG. 84 is a front perspective view of an oral device 2900. The oral device 2900 can be the same or similar in structure and/or function to any of the oral devices described herein, such as the oral device 100 and/or the oral device 2300. The oral device 2900 includes a single bite block 2920 and a single cheek protector 2914 to engage one side of a user’s mouth. In some embodiments, the oral device 2900 can include a handle 2960. In some embodiments, the bite block 2920 and/or cheek protector 2914 can be angled and/or tapered to accommodate the jaw angle of the user when the oral device 2900 is disposed in the user’s mouth. In some embodiments, the bite block 2920 can be configured to keep the user’s mouth open when the oral device 2900 is disposed in the mouth of the user. In use, the oral device 2900 can prevent excessive movement in the user’s mouth that may occur during an episode of movement disorder (e.g., during seizure). For example, the oral device 2900 can be disposed within the user’s mouth to prevent the user from inadvertently biting their tongue and/or prevent the user from moving their jaw (i.e., the user’s jaw can be secured by using oral device 2900). In some embodiments, as shown in FIG. 84, the cheek protector 2914 can extend laterally away from the bite block 2920 (e.g., a distance as wide or wider than the bite block 2920) to push the user’s buccal tissues (e.g., inner cheeks) away from the teeth. In some embodiments, the cheek protector 2914 defines a cavity within which absorbent material (e.g., cotton balls) can be disposed. Although shown as extending away from the proximal portion of the oral device 2900, the handle 2960 can extend away from the distal portion of the oral device 2900. In use, the oral device 2900 can allow the user to swallow (e.g., swallow saliva) when the oral device 2900 is disposed within the user’s mouth.

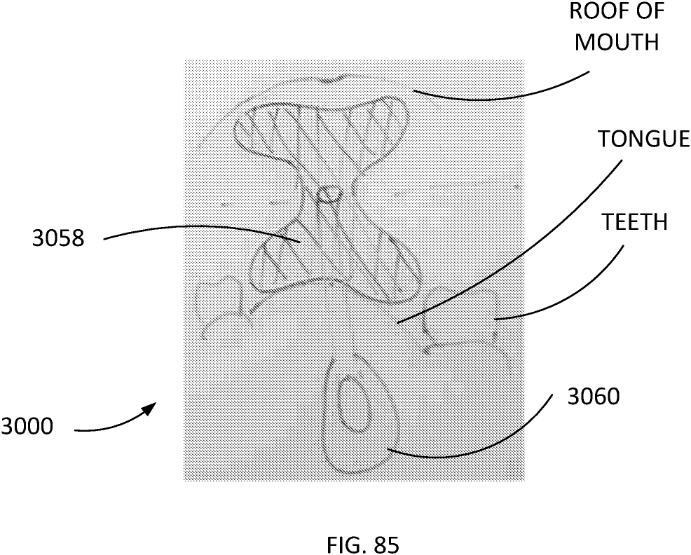

FIG. 85 is a front view of an oral device 3000. The oral device 3000 can be the same or similar in structure and/or function to any of the oral devices described herein, such as the oral device 100 and/or the oral device 2800. The oral device 3000 includes a tongue depressor 3058. The oral device 3000 can include a handle 3060. The tongue depressor 3058 can be configured such that, when the oral device 2400 is disposed within the user’s mouth, the tongue depressor 3058 can apply pressure on the superior surface of the user’s tongue to urge the user’s tongue towards the user’s lower jaw and away from the user’s bite plane. The tongue depressor 3058 can be configured to be disposed in the user’s mouth such that an upper surface of the tongue depressor 3058 engages the roof of the user’s mouth and a lower surface of the tongue depressor 3058 engages the user’s tongue. In use, the tongue depressor 3058 applies pressure to the lower jaw (via pressure on the tongue) and applies pressure to the upper jaw (via pressure on the roof of the mouth) such that the upper teeth of the user are unable to contact the lower teeth of the user. The force of the roof of the user’s mouth and the user’s tongue against the upper and lower surface of the tongue depressor 3058 causes the oral device 3000 to be retained within the user’s mouth prevents the oral device 3000 from being inadvertently dislodged from the user’s mouth. The upper surface and the lower surface of the tongue depressor 3058 can be formed as any suitable shape to engage the tongue and roof of the user’s mouth. In some embodiments, the upper surface and the lower surface of the tongue depressor 3058 are formed to be the same shape such that a user or caregiver can optionally insert the oral device 3000 into the user’s mouth with the upper surface or the lower surface of the tongue depressor 3058 contacting the user’s tongue and the other of the upper surface or the lower surface of the tongue depressor 3058 contacting the roof of the user’s mouth. For example, the upper surface and the lower surface of the tongue depressor 3058 can each be concave such that either surface can receive the surface of the user’s tongue. In some embodiments, when the user applies force against the tongue depressor 3058, the tongue depressor 3058 may compress from an expanded configuration to a compressed configuration, but may not expand laterally into the space between the user’s upper and lower teeth. In some embodiments, the tongue depressor 3058 may be biased toward the expanded configuration.

The oral device 3000 can be sufficiently large (e.g., have a sufficient extent or maximum overall distance between the upper surface and the lower surface of the tongue depressor 3058) such that a user is unable to swallow the oral device 3000. In some embodiments, the tongue depressor 3058 can be angled and/or tapered from a distal end to a proximal end to accommodate the jaw angle of the user when the oral device 3000 is disposed in the user’s mouth. In some embodiments, the handle 3060 is sufficiently rigid such that the handle 3060 can be used to insert the oral device 3000 into the user’s mouth (i.e., by pushing) and to remove the oral device 3000 from the user’s mouth (i.e., by pulling). In use, the oral device 3000 can allow the user to swallow (e.g., swallow saliva) when the oral device 3000 is disposed within the user’s mouth.

In some embodiments, an oral device, such as any of the oral devices described herein, can be used to deliver medicament (e.g., medication) to a user. For example, an oral device can contain and/or be coated with medicament that can be dispensed to a user after the oral device is disposed within the user’s mouth. An oral device can be used to deliver medicament in the form of a gel capsule, an aqueous solution, a powdered drug, a drug cake, a drug paste, a tablet or capsule pill, a liquid formulation, and/or any other suitable form of medicament. When an oral device is disposed in the mouth of a user, the medicament can be configured to be delivered to the user automatically (e.g., via the dissolving of a barrier containing medicament), as a result of an action by the user (e.g., the user biting down on the oral device to release the medicament), and/or as a result of an action by a clinician or caregiver (e.g., pushing on a portion of the oral device to release the medicament). In some embodiments, an oral device capable of delivering medicament is intended to be a single-use oral device, such that the oral device contains a finite quantity of medicament and is not intended to be refilled and/or recoated. In some embodiments, the oral device capable of delivering medicament is intended to be reusable, such that after the oral device has delivered medicament to the user, an additional quantity of medicament can be received by the oral device for a subsequent use of the oral device to deliver medicament to the user.

The oral devices described herein can be used to administer medicament to a user experiencing a variety of medical problems or symptoms. For example, the oral devices described herein can be used to administer medicament to a user experiencing dysphagia, which is experienced as a primary issue and/or a secondary issue or side effect associated with a primary issue. A user may experience dysphagia due to, for example, age, a stroke, a transient ischemic attack (TIA), hemiparesis of the tongue, epilepsy and seizure disorders, traumatic brain injury, cancer, tardive dyskinesia, dysmotility, muscular dystrophy, myotonic dystrophy, cerebral palsy, Parkinson’s Disease, and/or other degenerative neurological disorders such as amyotrophic lateral sclerosis (ALS), multiple sclerosis (MS), progressive supranuclear palsy, Huntington’s disease, and/or myasthenia gravis. Furthermore, a user may accidentally aspirate food, liquid, or medicament, which can cause the user to cough, choke, and/or build up excess saliva in the user’s mouth. Additionally, some users may experience difficulty taking medication due to xerostomia (i.e., dry mouth) and/or a psychological aversion leading to the patient believing that they cannot swallow the medication. The oral devices described herein can be used to administer medicament to user’s experiencing difficulty swallowing medication (e.g., to help the user adhere to a prescribed medication regimen). In some embodiments, the oral devices described herein can be used to administer a medicament quickly (e.g., for seizure or stroke treatment) such that medicament is swallowed via one swallow or absorbed by the user’s body in a short period of time (e.g., a number of seconds or 1-2 minutes). In some embodiments, the medicament can be formed as a pill intended for swallowing. In some embodiments, the pill can include, for example, clonazepam, lorazepam, alprazolam, diazepam, and/or other emergency anti-epileptic drugs used to treat (e.g., stop) a seizure. In some embodiments, a distal end of a catheter can be inserted through a distal opening of an oral device described herein to deliver water to the user to assist in swallowing a medicament (e.g., in the form of a tablet or a capsule pill). In some embodiments, the oral devices described herein can be used to administer a medicament over a prolonged predetermined administration period such that the medicament is received by the patient passively.

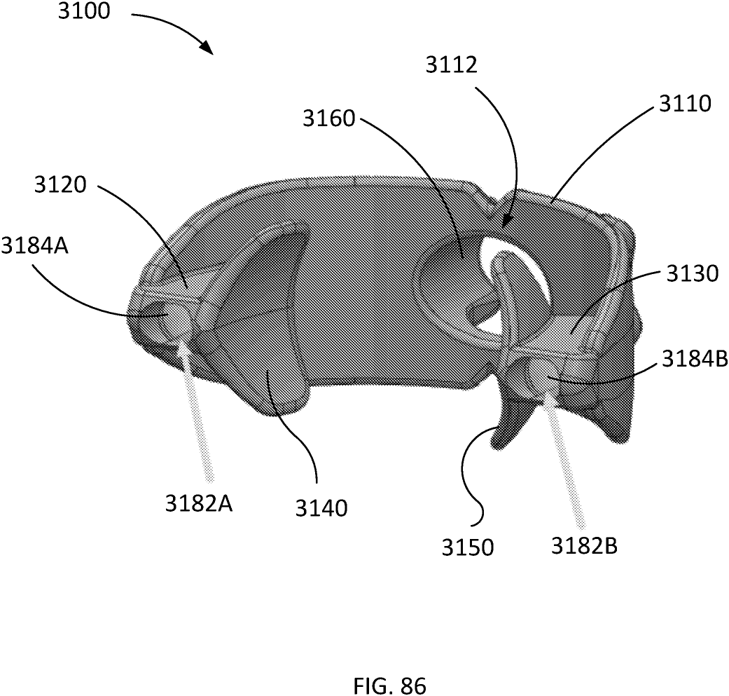

FIG. 86 is a perspective view of an oral device 3100. The oral device 3100 can be the same or similar in structure and/or function to any of the oral devices described herein, such as, for example, the oral device 300 and/or the oral device 900. For example, the oral device 3100 includes a curved frame 3110, a first bite block 3120, a second bite block 3130, a first wing 3140, and a second wing 3150. The oral device 3100 also includes a handle 3160. The curved frame 3110 defines an opening 3112. The curved frame 3110, the first bite block 3120, the second bite block 3130, the first wing 3140, and the second wing 3150 can be the same or similar in structure and/or function to any of the curved frames, the first bite blocks, the second bite blocks, the first wings, and the second wings, respectively, described herein.

The first bite block 3120 and the second bite block 3130 define a first pocket 3182A and a second pocket 3182B, respectively. As shown in FIG. 86, the first pocket 3182A and the second pocket 3182B can be open toward a proximal end of the first bite block 3120 and the second bite block 3130, respectively. Alternatively or additionally, the first pocket 3182A and the second pocket 2382B can be open toward a distal end of the first bite block 3120 and the second bite block 3130. In some embodiments, the first pocket 3182A and the second pocket 3182B can be sized to receive medicament.

In some embodiments, the first bite block 3120 and the second bite block 3130 can include a first frangible cover 3184A and a second frangible cover 3184B, respectively. The first frangible cover 3184A can form a boundary of the first pocket 3182A and the second frangible cover 3184B can form a boundary of the second pocket 3182B. The first frangible cover 3184A can be configured to open (e.g., rupture) such that the contents of the first pocket 3182A are released into the user’s mouth due to a user biting on the first bite block 3120. The second frangible cover 3184B can be configured to open (e.g., rupture) such that the contents of the second pocket 3182B are released into the user’s mouth due to the user biting on the second bite block 3130.

In some embodiments, the medicament disposed within the first pocket 3182A and the second pocket 3182B can be in the form of a gel cap, an aqueous solution, and/or a powdered drug. In some embodiments, such as when the medicament is in the form of a gel cap, the first bite block 3120 and the second bite block 3120 can not include the first frangible cover 3184A and the second frangible cover 3184B, respectively, and the first pocket 3182A and the second pocket 3182B can instead be open to an exterior of the oral device 3100.

Although not shown, in some embodiments, rather than both of the first bite block 3120 and the second bite block 3130 defining a pocket to receive and retain medicament, only one of the first bite block 3120 and the second bite block 3130 can define a pocket to receive and retain medicament.

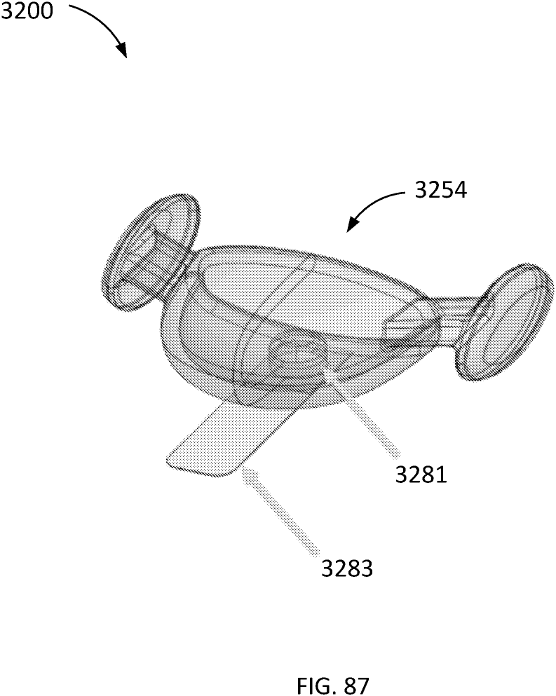

FIG. 87 is a front perspective view of an oral device 3200. The oral device 3200 can be the same or similar in structure and/or function to any of the oral devices described herein, such as the oral device 400, oral device 700, and/or the oral device 800. For example, the oral device 3200 can include a tongue protector 3254 (e.g., a tongue protector similar in structure and/or function to the tongue protector 454). The tongue protector 3254 defines a cavity 3281 in a bottom surface of the tongue protector 3254. The cavity 3281 is shaped and sized to receive medicament (e.g., a pill). The oral device 3200 also includes a removable strip 3283 configured to be disposed over the cavity 3281 to enclose the medicament within the cavity 3281. In some embodiments, the strip 3283 is formed monolithically with the tongue protector 3254. For example, a first end of the strip 3283 can be coupled to the tongue protector 3254 (e.g., via a living hinge) and a second end of the strip 3283 can be free. The second end can be displaced such that the strip 3283 is shifted away from the cavity 3281 (e.g., via rotation about the first end of the strip 3283 or peeling of the strip 3283 from the first end to the second end) and the medicament can fall from the cavity 3281. In some embodiments, the strip 3283 is coupled to the tongue protector 3254 via an adhesive and fully separable from the tongue protector 3254 such that medicament can fall from the cavity 3281. In use, the oral device 3200 can be inserted into a user’s mouth. The strip 3283 can then be pulled and/or peeled to displace the strip 3283 relative to the cavity 3281 such that the medicament falls into the sublingual space of the user’s mouth.

In some embodiments, the strip 3283 is formed of a dissolvable material that dissolves upon interaction with saliva such that the strip 3283 no longer obstructs the opening to the cavity 3281 and the medicament is released. In some embodiments, the strip 3283 is formed of a dissolvable material and is also coupled to the tongue protector 3254 via the adhesive such that the medicament can fall from the cavity 3281 via the strip 3283 dissolving with saliva and/or the strip 3283 being pulled and/or peeled to displace the strip 3283 relative to the cavity 3281. In some embodiments, the strip 3283 is formed of the dissolvable material and is also coupled to the tongue protector 3254 via the first end of the strip 3283 such that the medicament can fall from the cavity 3281 via the strip 3283 dissolving with saliva and/or the second end of the strip 3283 being displaced such that the strip 3283 is shifted away from the cavity 3281. In some embodiments, the strip 3283 is coupled to the tongue protector 3254 via the adhesive and the strip 3283 is also coupled to the tongue protector 3254 via the first end of the strip 3283 such that the medicament can fall from the cavity 3281 via the strip 3283 being pulled and/or peeled to displace the strip 3283 relative to the cavity 3281 and/or the second end of the strip 3283 being displaced such that the strip 3283 is shifted away from the cavity 3281. In some embodiments, the strip 3283 is formed of a dissolvable material, the strip 3283 is coupled to the tongue protector 3254 via the adhesive, and the strip 3283 is also coupled to the tongue protector 3254 via the first end of the strip 3283 such that the medicament can fall from the cavity 3281 via the strip 3283 dissolving with saliva, the strip 3283 being pulled and/or peeled to displace the strip 3283 relative to the cavity 3281, and/or the second end of the strip 3283 being displaced such that the strip 3283 is shifted away from the cavity 3281.

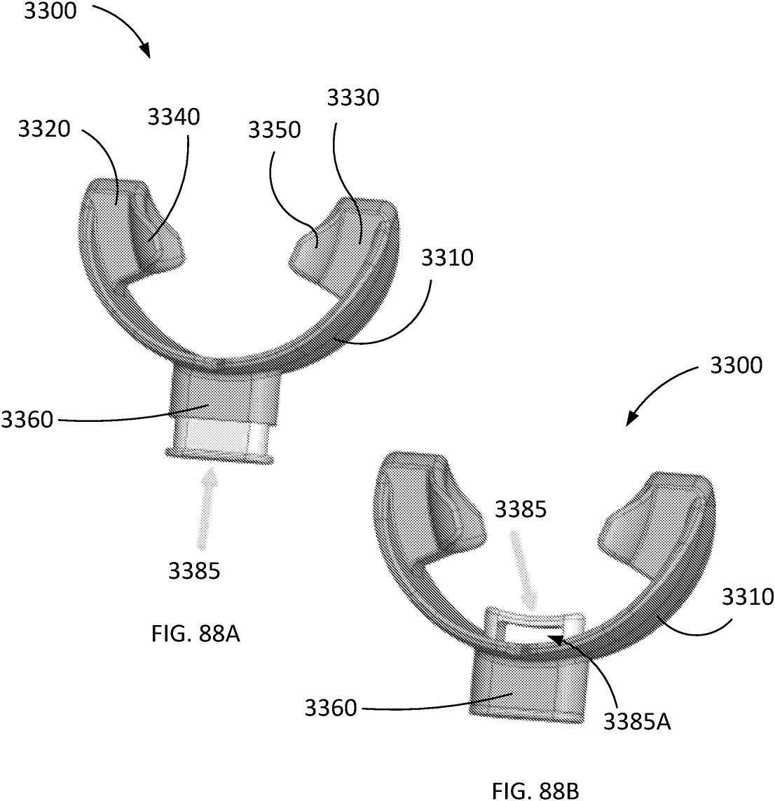

FIGS. 88A and 88B are perspective views of an oral device 3300 in a first configuration and a second configuration, respectively. The oral device 3300 can be the same or similar in structure and/or function to any of the oral devices described herein, such as the oral device 300 and/or the oral device 900. For example, the oral device 3300 includes a curved frame 3310, a first bite block 3320, a second bite block 3330, a first wing 3340, and a second wing 3350. The curved frame 3310, the first bite block 3320, the second bite block 3330, the first wing 3340, and the second wing 3350 can be the same or similar in structure and/or function to any of the curved frames, the first bite blocks, the second bite blocks, the first wings, and the second wings, respectively, described herein.

As shown in FIGS. 88A and 88B, the oral device 3300 also includes a handle 3360 and a drawer receptacle 3385. The handle 3360 defines a through-hole configured to receive the drawer receptacle 3385. The drawer receptacle 3385 defines a compartment configured to receive and retain medicament. In some embodiments, the medicament disposed within the drawer receptacle 3385 can be in the form of a gel cap, an aqueous solution, and/or a powdered drug. The drawer receptacle 3385 includes a distal closed first end and a proximal closed second end opposite the first end. The drawer receptacle 3385 also defines at least one opening 3385A in a sidewall of the drawer receptacle 3385 (e.g., in one or both of an upper and lower surface of the drawer receptacle 3385). The oral device 3300 can be transitioned (e.g., pushed) from a first configuration, as shown in FIG. 88A, in which the drawer receptacle 3385 is in a closed first position relative to the handle 3360, to a second configuration, as shown in FIG. 88B, in which the drawer receptacle 3385 is in an open second position relative to the handle 3360. The drawer receptacle 3385 can be sufficiently long from the first end to the second end and the opening 3385A sufficiently long such that, when the drawer receptacle 3385 is disposed in the second position, medicament can pass through the opening 3385A. Thus, when the drawer receptacle 3385 is in the first position, a medicament can be retained within the compartment of the drawer receptacle 3385, and when the drawer receptacle 3385 is in the second position, the medicament can pass through one of the openings 3385A to an exterior of the oral device 3300. In some embodiments, the height of the drawer receptacle 3385 can be the same or less than the height of the first bite block 3320 and the second bite block 3330 such that the drawer receptacle 3385 can translate into a gap defined between upper front teeth and lower front teeth of the user when the oral device 3300 is disposed in the user’s mouth.

In some embodiments, the drawer receptacle 3385 can be separated from the handle 3360 (e.g., after the drawer receptacle 3385 is disposed in the second position and the medicament falls through the opening 3385A). For example, the drawer receptacle 3385 can be pulled distally and separated from the handle 3360 leaving the through-hole defined by the handle 3360 unobstructed such that the through-hole can be used for fluid aspiration and/or delivery. For example, a suction catheter can be translated through the through-hole from a first side of the curved frame 3310 to a second side of the curved frame 3310.

In some embodiments, rather than the drawer receptacle 3385 having an outer perimeter corresponding to an inner perimeter of the handle 3360 as shown in FIG. 88A, the drawer receptacle 3385 can have a smaller outer perimeter such that fluid and/or a catheter (e.g., a suction catheter) can be passed between the outer surface of the drawer receptacle 3385 and the inner surface of the handle 3360. In some embodiments, rather than the handle 3360 and the drawer receptacle 3385 being centered relative to the curved frame 3310, the drawer receptacle 3385 can be disposed off-center and the curved frame 3310 can define another opening configured to receive a catheter and/or allow for fluid to flow through the curved frame 3310.

In use, the oral device 3300 can be inserted into a user’s mouth. For example, the user or a caregiver can insert the oral device 3300 into the user’s mouth by gripping the handle 3360 and inserting the oral device 3300 between the user’s upper and lower teeth such that the first bite block 3320 and the second bite block 3330 can be engaged by the user’s molars and/or premolars. The drawer receptacle 3385 can be transitioned from the first position to the second position such that the medicament drops into the user’s mouth (e.g., the sublingual space). In some embodiments, the drawer receptacle 3385 can optionally be pulled out of engagement with the handle 3360 and set aside. In some embodiments, holes or apertures are define on the curved frame 3310 such that oral fluids and air can pass through the holes or apertures and a suction catheter can be administered through one of the holes or apertures on the curved frame 3310.

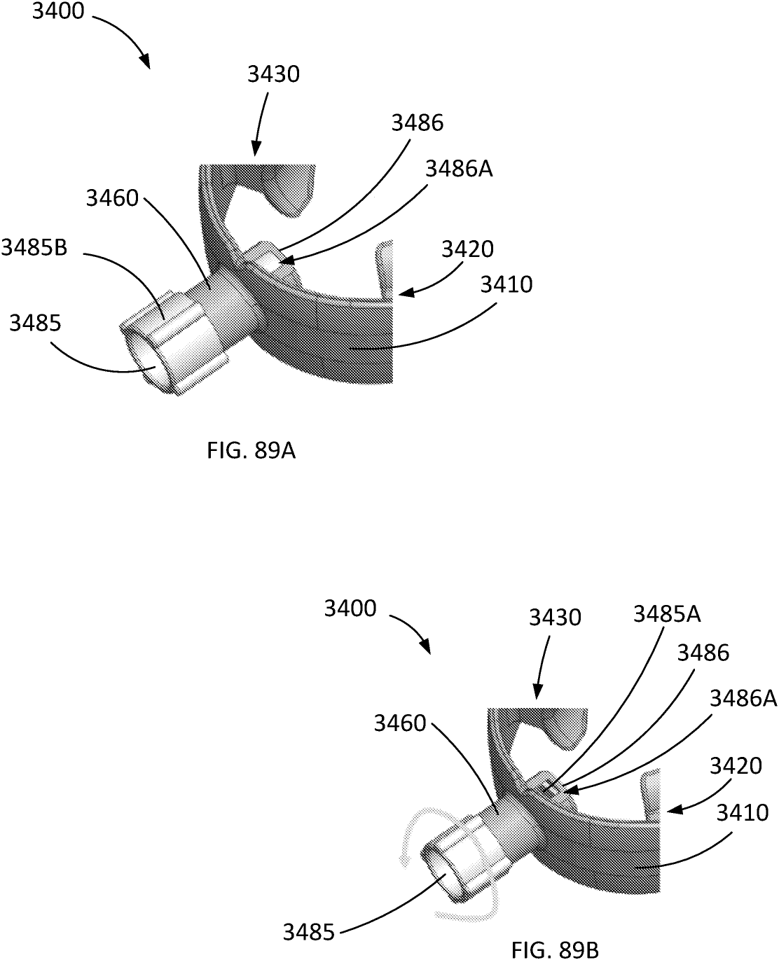

FIGS. 89A and 89B are perspective views of a portion of an oral device 3400. The oral device 3400 can be the same or similar in structure and/or function to any of the oral devices described herein, such as the oral device 300, oral device 900 and/or oral device 3300. For example, the oral device 3400 includes a curved frame 3410, a handle 3460 (also referred to as a tubular portion), a first bite block 3420, and a second bite block 3430. The oral device 3400 also includes a dispenser portion 3486 and a cylindrical receptacle 3485 that defines a compartment configured to receive and retain a medicament. In some embodiments, the medicament disposed within the cylindrical receptacle 3485 can be in the form of a gel cap, a pill, an aqueous solution, a powdered drug, and/or any other suitable formulation. The handle 3460 and the dispenser portion 3486 collectively define an interior space configured to receive at least a portion of the cylindrical receptacle 3485.

The dispenser portion 3486 is coupled to and extends proximally from the curved frame 3410 opposite the handle 3460. The dispenser portion 3486 includes a first end coupled to the curved frame 3410 and a second end opposite the first end and disposed proximal of the first end. The interior space collectively defined by the handle 3460 and the dispenser portion 3486 is cylindrically shaped such that the cylindrical receptacle 3485 can rotate within the interior space and the outer surface of the cylindrical receptacle 3485 within the interior space is in contact with the inner surface of the handle 3460 and the dispenser portion 3486. In some embodiments, the interior cavity can be open to the second end of the dispenser portion 3486 such that the handle 3460 and the dispenser portion 3486 define a through-hole from an area on a first side of the curved frame 3410 to an area on the second side of the curved frame 3410 when the interior cavity is unobstructed by the cylindrical receptacle 3485. In some embodiments, the dispenser portion 3486 has a height such that when the oral device 3400 is disposed in the mouth of a user, the user can engage the dispenser portion 3486 with their front teeth (e.g., by biting on the dispenser portion 3486). In some embodiments, the dispenser portion 3486 has a height such that when the oral device 3400 is disposed in the mouth of a user, the user cannot engage the dispenser portion 3486 with their front teeth. For example, the first bite block 3420 and the second bite block 3430 can be sufficiently thick (e.g., thicker than the height of the dispenser portion 3486) to maintain the front teeth away from the dispenser portion 3486. The dispenser portion 3486 defines at least one opening 3486A in a portion of the sidewall of the dispenser portion 3486 between the curved frame 3410 and the second end of the dispenser portion 3486 sized such that medicament can pass from the interior cavity of the dispenser portion 3486 and the handle 3460 to an exterior of the dispenser portion 3486 (e.g., into a user’s mouth) via at least one opening 3486A.

The cylindrical receptacle 3485 includes a distal first end and a proximal closed second end opposite the first end. In some embodiments, the distal first end of the cylindrical receptacle 3485 can be a closed end defining a distal boundary of the compartment of the cylindrical receptacle 3485. In some embodiments, an internal wall disposed between the first end and the second end defines a distal boundary of the compartment of the cylindrical receptacle 3485. In some embodiments, the cylindrical receptacle 3485 or a portion of the cylindrical receptacle 3485 extending distally from the curved frame 3410 is rotationally symmetrical about an axis extending from the distal closed first end and the proximal closed second end. The cylindrical receptacle 3485 also defines at least one opening 3485A in a sidewall of the cylindrical receptacle 3485 (e.g., a portion of the sidewall of the cylindrical receptacle 3485 between the curved frame 3410 and the second end of the cylindrical receptacle 3485). When the cylindrical receptacle 3485 is disposed within the interior cavity and the opening 3485A of the cylindrical receptacle 3485 and the opening 3486A are aligned, medicament can pass unobstructed from the compartment of the cylindrical receptacle 3485 through both the opening 3485A and opening 3486A. When the opening 3485A and opening 3486A are not aligned, medicament is obstructed from traveling from the compartment of the cylindrical receptacle 3485 through the opening 3485A by the sidewall of the dispenser portion 3486.

In some embodiments, as shown in FIGS. 89A and 89B, the cylindrical receptacle 3485 includes a handle portion 3485B that can be gripped by a user or caregiver (e.g., via gripping ribbed portions on the handle portion 3485B) to rotate the cylindrical receptacle 3485 within the interior cavity described by the handle 3460 and the dispenser portion 3486. The handle portion 3485B can be cylindrical and can be coupled to the first end of the handle 3460. The cylindrical receptacle 3485 can be transitioned (e.g., via rotating and/or twisting the handle portion 3485B) relative to the handle 3460 and the dispenser portion 3486 between a closed first configuration, as shown in FIG. 89A, in which the opening 3485A of the cylindrical receptacle 3485 is not aligned with the opening 3486A of the dispenser 3486, and an open second configuration, as shown in FIG. 89B, in which the opening 3485A of the cylindrical receptacle 3485 is aligned with the opening 3486A of the dispenser 3486. The opening 3485A of the cylindrical receptacle 3485 and the opening 3486A of the dispenser 3486 can be sufficiently wide such that, when the oral device 3400 is in the open configuration, medicament can pass through the opening 3485A and the opening 3486A. Thus, when the cylindrical receptacle 3485 is in the closed configuration, a medicament can be retained within the compartment of the cylindrical receptacle 3485, and when the cylindrical receptacle 3485 is in the open configuration, the medicament can pass through the opening 3485A and the opening 3486A to an exterior of the oral device 3400. In some embodiments, the dispenser portion 3486 can define a first opening 3486A and a second opening 3486A in opposing positions in a top and bottom side of the dispenser portion 3486 such that the oral device 3400 is symmetrical about a plane extending through the handle 3460, the curved frame 3410, the first bite block 3420, and the second bite block 3430 and can be inserted into a user’s mouth with the first bite block 3420 engaged with the left side of a user’s teeth and the second bite block 3430 engaged with the right side of the user’s teeth, or vice versa, and the medicament will fall into the user’s mouth when the cylindrical receptacle 3485 is in the open second configuration. In some embodiments, the cylindrical receptacle 3485 can include a first opening 3485A and a second opening 3485A in opposing sides of the cylindrical receptacle 3485 such that either of the openings 3485A can be rotated into engagement with an opening 3486A such that the medicament falls out of the compartment and into the user’s mouth.

In use, the oral device 3400 can be inserted into a user’s mouth. For example, the user or a caregiver can insert the oral device 3400 into the user’s mouth by gripping the handle 3460 and inserting the oral device 3400 between the user’s upper and lower teeth such that the first bite block 3420 and the second bite block 3430 can be engaged by the user’s molars and/or premolars. The cylindrical receptacle 3485 can be transitioned from the closed first configuration to the open second configuration such that the medicament can travel through the opening 3485A and the opening 3486A into the user’s mouth.

In some embodiments, the cylindrical receptacle 3485 can optionally be separated from (e.g., pulled distally out of engagement with) the handle 3460 and the dispenser portion 3486 and set aside leaving the through-hole defined by the handle 3460 and dispenser portion 3486 unobstructed such that the through-hole can be used for fluid aspiration and/or delivery. For example, a suction catheter can be translated through the through-hole from a first side of the curved frame 3410 to a second side of the curved frame 3410 (e.g., into the user’s mouth). In some embodiments, the handle 3460 and dispenser portion 3486 can be sized such that multiple through-holes can be defined within the handle 3460 and the dispenser 3486. For example, in some implementations, a first through-hole can be used to receive a first cylindrical receptacle 3485 and a second through-hole can be used to receive a second cylindrical receptacle 3485. In some implementations, a second through-hole can be configured to receive a catheter and/or allow for fluid to flow through the curved frame 3410. In some embodiments, the second end of the dispenser portion 3486 can be closed. In some embodiments, rather than the handle 3460 and the dispenser portion 3486 being centered relative to the curved frame 3410, the dispenser 3486 can be disposed off-center. In some embodiments, a set of one or more holes or apertures can be defined by the curved frame 3410 such that oral fluids and air can pass through the holes or apertures and a suction catheter can be administered through one of the holes or apertures on the curved frame 3410.

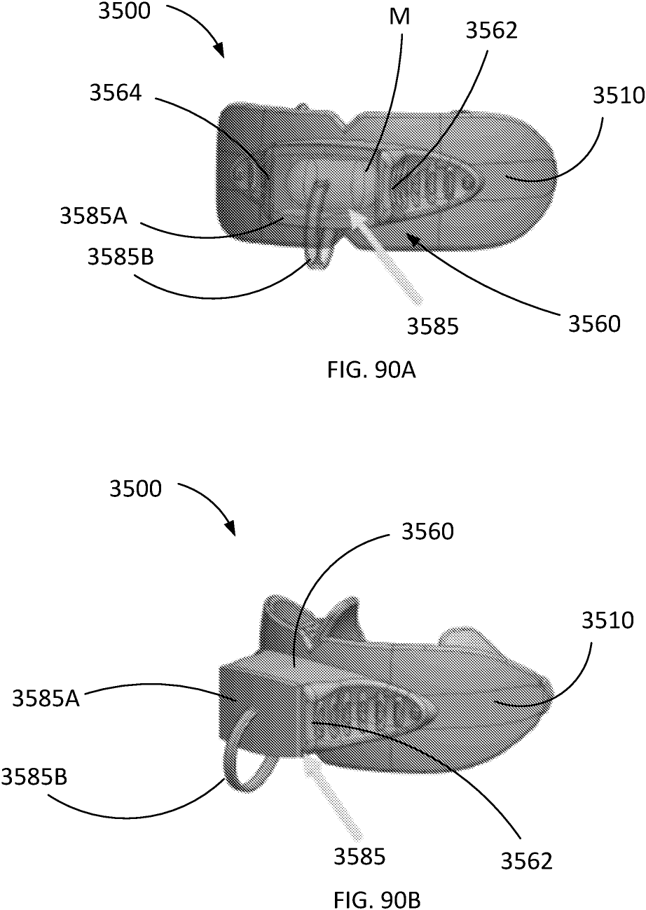

FIGS. 90A and 90B are perspective views of an oral device 3500. The oral device 3500 can be the same or similar in structure and/or function to any of the oral devices described herein, such as the oral device 300 and/or oral device 900. For example, the oral device 3500 includes a curved frame 3510 and a handle 3560. In some embodiments, the handle 3560 includes a first handle portion 3562 and a second handle portion 3564 each extending distally away from the curved frame 3510. The oral device 3500 also includes a receptacle 3585 that defines a compartment configured to receive and retain a medicament M. In some embodiments, the medicament M disposed within the receptacle 3585 can be in the form of a gel cap, a pill, an aqueous solution, a powdered drug, and/or any other suitable formulation. The receptacle 3585 includes a distal open first end and a proximal closed second end opposite the first end. In some embodiments, the medicament M can be disposed within the receptacle 3585 by inserting the medicament M through the open first end of the receptacle 3585. In some embodiments, the second end of the lidded receptacle 3585 is coupled to the curved frame 3510. In some embodiments, the lidded receptacle 3585 is disposed between the first handle portion 3562 and second handle portion 3564. In some embodiments, the receptacle 3585 is disposed between the first handle portion 3562 and second handle portion 3564 such that the receptacle 3585, the first handle portion 3562, and second handle portion 3564 collectively form the handle 3560 and/or collectively define an interior compartment of the receptacle 3585.

The oral device 3500 includes a lid 3585A that can be transitioned between a closed position in which the lid 3585A forms a boundary of the interior compartment of the receptacle 3585 such that the receptacle 3585 is closed and an open position in which the lid 3585A does not obstruct the open end of the receptacle 3585 and the medicament M is accessible through the open first end. In the open configuration of the receptacle 3585, the lid 3585 does not prevent medicament M from passing through the first end of the receptacle 3585 (e.g., for medicament M to enter the receptacle 3585 or for medicament M to be removed from the receptacle 3585). In the closed configuration of the receptacle 3585, the lid 3585 can prevent medicament M from passing through the first end of the receptacle 3585 (e.g., to retain the medicament M within the lidded receptacle 3585). In some implementations, the closed configuration of the receptacle 3585 can prevent fluid from passing through the first end of the lidded receptacle 3585. In some embodiments, the lid 3585A can be coupled to the receptacle 3585 (e.g., adjacent to or near the first end) and can transition between the open and closed positions using any suitable mechanism, such as a living hinge or a spring. For example, the lid 3585A can form a door attached via a hinge to the first end of the receptacle 3585, such that when the door is open the lidded receptacle 3585 is the open configuration and when the door is closed the receptacle 3585 is in the closed configuration. In some embodiments, the lid 3585A can be temporarily coupled to the first end of the receptacle 3585 in the closed configuration, and the lid 3585A can be decoupled from the first end of the receptacle 3585 by separating the lid 3585A from the receptacle 3585 to transition the receptacle 3585 to the open configuration. For example, in some implementations, the lid 3585A can be made to couple/decouple from the first end of the receptacle 3585 using any suitable mechanism, such as a friction fit, a lock, an adhesive, a frangible seal, or a screw thread. In some embodiments, the receptacle 3585 includes a tether 3585B coupling the lid 3585A to the receptacle 3585 such that if the lid 3585A is decoupled from the first end of the receptacle 3585, the lid 3585A remains coupled to the oral device 3500 via the tether 3585B. For example, the tether 3585B can be a cable, a string, a plastic strip, a ribbon, and/or any other suitable structure.

In use, the oral device 3500 can retain a medicament M that can be administered to a user when the oral device 3500 is disposed outside the user’s mouth. For example, prior to inserting the oral device 3500 in the user’s mouth (or after removing the oral device 3500 from the user’s mouth), the user or caregiver can remove the lid 3585A from the receptacle 3585 and can remove the medicament M from the receptacle 3585. The medicament M can then be orally administered to the user. Thus, the oral device 3500 can be a container and/or packaging mechanism for the medicament M, which can increase convenience for the user and/or caregiver (e.g., the user and/or caregiver will not need to account for a separate container of medicament M). In some embodiments, a set of one or more holes or apertures can be defined by the curved frame 3510 such that oral fluids and air can pass through the holes or apertures and a suction catheter can be administered through one of the holes or apertures on the curved frame 3510.

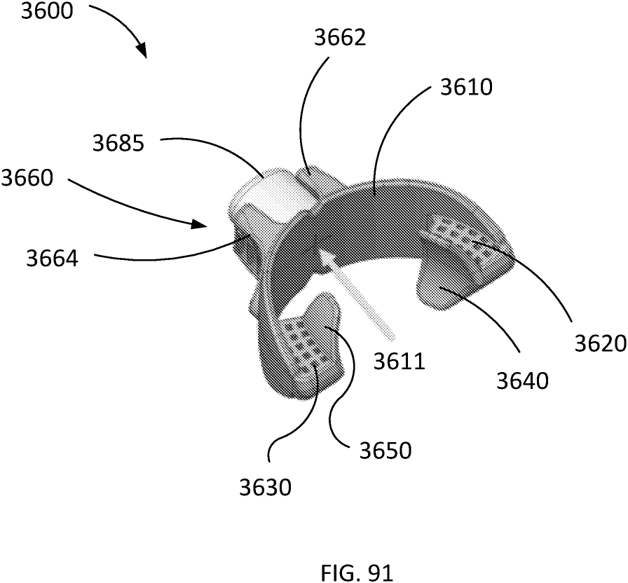

FIG. 91 is a perspective view of an oral device 3600. The oral device 3600 can be the same or similar in structure and/or function to any of the oral devices described herein, such as the oral device 300, the oral device 900, and/or oral device 3500. For example, the oral device 3600 includes a curved frame 3610, a first bite block 3620, a second bite block 3630, a first wing 3640, a second wing 3650, and a handle 3660. The handle 3660 includes a first handle portion 3662 and a second handle portion 3664 each extending distally away from the curved frame 3610. The oral device 3600 also includes a removable canister 3685 that defines a compartment configured to receive and retain a medicament. In some embodiments, the medicament disposed within the canister 3685 can be in the form of a gel cap, a pill, an aqueous solution, a powdered drug, and/or any other suitable formulation. The canister 3685 includes a distal closed first end and a proximal open second end opposite the first end. In some embodiments, the second end of the canister 3685 can couple to the curved frame 3610. For example, the canister 3685 can couple to the curved frame such that the canister 3685 is disposed between the first handle portion 3662 and second handle portion 3664. The canister 3685 can be configured to couple with the curved frame 3610 using any suitable mechanism (e.g., threaded screw, friction fit, etc.) such that the canister 3585 will not decouple from the curved frame 3610 when the oral device 3600 is disposed in a user’s mouth and the oral device 3600 is subjected to intemal forces (e.g., biting forces) and external forces (e.g., external contact forces) that may manifest during periods of uncontrolled user movements (e.g., seizures). The canister 3685, however, can be configured to decouple from the curved frame 3610 when required by a user or caregiver (e.g., to add medicament to the canister 3685) using any suitable mechanism (e.g., unscrewing the canister 3685 from the curved frame 3610 or pulling the canister 3685 distally relative to the curved frame 3610). In some alternative embodiments, the canister 3685 is permanently coupled to the curved frame 3610 but still allows for medicament or other substances to enter and/or exit the canister 3685 (e.g., for retainment of medicament or administration of medicament). For example, the canister 3685 can include a door that allows for a user to insert and retain a medicament within the canister 3685. As another example, the canister 3685 can be configured for medicament and/or other substances to enter and/or exit the canister 3685 via an opening aligned with a valve 3611 defined by the curved frame 3610. For example, the canister 3685 can be pre-filled and coupled to the curved frame 3610 such that the canister 3685 is retained by the first handle portion 3662 and the second handle portion 3664.

The curved frame 3610 can include a valve 3611 on the portion of the curved frame 3610 that is made to couple with the canister 3685 such that medicament or other substances can pass through the second end of the canister 3685 and through the curved frame 3610 via the valve 3611 (e.g., to enter the user’s oral cavity when the oral device 3600 is disposed on in the user’s mouth). The valve 3611 can be transitioned between an open configuration, in which medicament or other substances can pass through the valve 3611, and a closed configuration, in which medicament or other substances cannot pass through the valve 3611 and remain disposed within the canister 3685. For example, the valve 3611 can include a set of flexible flaps (e.g., four flaps) that can be transitioned from the closed configuration shown in FIG. 91 in which the flaps are disposed in the same plane to an open configuration in which the flaps are bent away from the canister 3685 to define an opening through which medicament can pass from the canister 3685 through the curved frame 3610. In some embodiments, the flaps can be coupled to one another via a frangible seal in the closed configuration that can be severed to transition the flaps to the open configuration. The valve 3611 can be transitioned to the open configuration under a force applied by the canister 3685 to the flaps of the valve 3611 to bend the flaps away from one another to define the opening. For example, the canister 3685 can be sufficiently pliable such that when a user or caregiver exerts an external force upon the canister 3685 (e.g., by pushing the first end of canister 3685 towards the curved frame 3610 or opposing sidewalls of the canister 3685 toward each other) to increase the pressure within the canister 3685, the contents of the canister can be urged toward the valve 3611 (e.g., via an open end, valve, or opening in the canister 3685), apply the transitioning force to the valve 3611, and be urged through the opening defined by the valve 3611. In some embodiments, the canister 3685 can include an activation button that can be pressed to initiate an increase in pressure within the canister 3685 to push medicament through the valve 3611 (e.g., the canister 3685 can be an aerosol canister).

In some embodiments, the canister 3685 can include one or more openings or apertures within the canister 3685 (e.g., in the portion of the canister adjacent the valve 3611) to allow medicament (e.g., powder) to pass through from the canister 3685 through the curved frame 3610 and into the user’s mouth. For example, when the oral device 3600 is disposed in the mouth of a user, the user can inhale through the valve 3611 and draw the medicament (e.g., an inhalant) from the canister 3685 into the user’s mouth and/or lungs..

In use, the oral device 3600 can retain a medicament that can be administered to a user when the oral device 3600 is disposed within the user’s mouth. After inserting the oral device 3600 in the user’s mouth, the user or caregiver can transition the valve 3611 from the closed configuration to the open configuration to administer the medicament from within the canister 3685 into the user’s mouth. Thus, the oral device 3600 can be a container and/or packaging mechanism for the medicament, which can increase convenience for the user and/or caregiver (e.g., the user and/or caregiver will not need to account for a separate container of medicament). In some embodiments, a set of one or more holes or apertures can be defined by the curved frame 3610 such that oral fluids and air can pass through the holes or apertures and a suction catheter can be administered through one of the holes or apertures on the curved frame 3610.

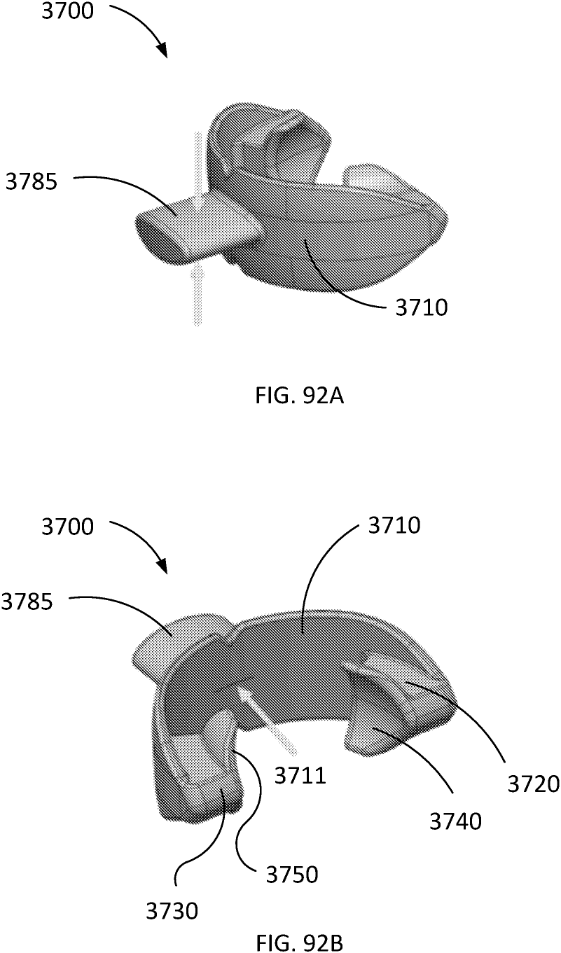

FIGS. 92A and 92B are perspective views of an oral device 3700. The oral device 3700 can be the same or similar in structure and/or function to any of the oral devices described herein, such as the oral device 300 and/or oral device 3600. For example, the oral device 3700 includes a curved frame 3710, a first bite block 3720, a second bite block 3730, a first wing 3740, and a second wing 3750. The oral device 3700 also includes a squeezable canister 3785 that includes pliable sidewalls and defines a compartment or reservoir configured to receive and retain a medicament. In some embodiments, the medicament disposed within the squeezable canister 3785 can be in the form of a gel cap, a pill, an aqueous solution, a powdered drug, and/or any other suitable formulation. The squeezable canister 3785 includes a distal closed first end and a proximal open second end opposite the first end and coupled to the distal surface of the curved frame 3710. In some embodiments, the squeezable canister 3785 can also function as a handle with similar functionality to any of the handles described herein, such as the handle 160 of the oral device 100 or the handle 460 of the oral device 400.

The curved frame 3710 can include a valve 3711 on the portion of the curved frame that couples with the squeezable canister 3785 such that medicament or other substances can pass through the second end of the squeezable canister 3785 and through the curved frame 3710 via the valve 3711 (e.g., to enter the user’s oral cavity). In some embodiments, medicament can only enter or exit the squeezable canister 3785 via the valve 3711. The valve 3711 can transition between a closed configuration, in which medicament or other substances cannot pass through the valve 3711 and remain disposed within the squeezable canister 3785 and an open configuration, in which medicament or other substances can pass through the valve 3711. In some embodiments, the medicament can be sealed within the squeezable canister 3785 such that an opening within the slit of the valve 3711 is not defined until a force is applied to the squeezable canister 3785 to increase the pressure within the squeezable canister 3785. For example, the slit can be sealed closed with a frangible seal. The frangible seal can be formed, for example, using a heat or ultrasonic seal. When the user or caregiver desires to administer the sealed medicament to the user, the user or caregiver can exert a sufficient squeezing force upon the squeezable canister 3785 to generate internal pressure sufficient to break the seal and define an opening within the slit of the valve 3711 such that medicament can pass through the slit and into the user’s mouth. In some embodiments, the valve 3711 can be biased towards the closed configuration such that the valve 3711 is normally closed (e.g., remains closed until manually opened by a user or caregiver). For example, the valve 3711 can include at least one slit that can be urged open to define an opening through the curved frame 3711, but the edges of the slit can be biased to be in apposition with each other such that the valve 3711 is normally closed. For example, the squeezable canister 3785 can be sufficiently pliable such that when a user or caregiver exerts an external force upon the canister 3685 (e.g., by squeezing the walls of the squeezable canister 3785), the pressure within the squeezable canister 3785 (i.e., pressure exerted from the enclosed fluid, medicament, or other substances within the squeezable canister 3785) can be a sufficient pressure to transition the valve 3711 from the closed configuration to the open configuration to allow medicament within the canister 3785 to flow through the valve (and thus the valve 3711 will return to the closed configuration when the pressure is not sufficient to open the valve 3711). In some embodiments, the squeezable canister 3785 can be sufficiently stiff such that the user or caregiver administering the medicament within the squeezable canister 3785 must exert a sufficient pressure upon the squeezable canister 3785 (i.e., when squeezing the squeezable canister 3785) for the valve 3711 to assume an open configuration. For example, the squeezable canister 3785 can be sufficiently stiff such that accidental contact forces (e.g., during the uncontrolled movements during a panic scenario) will not generate sufficient pressure to open the valve 3711 and dispense any medicament within the squeezable canister 3785.

In use, the oral device 3700 can retain a medicament that can be administered to a user when the oral device 3700 is disposed within the user’s mouth. After inserting the oral device 3700 in the user’s mouth, the user or caregiver can apply a squeezing force to the squeezable canister 3785 to transition the valve 3711 from the closed configuration to the open configuration to administer the medicament from within the squeezable canister 3785 into the user’s mouth. Thus, the oral device 3700 can be a container and/or packaging mechanism for the medicament, which can increase convenience for the user and/or caregiver (e.g., the user and/or caregiver will not need to account for a separate container of medicament). In some embodiments, a set of one or more holes or apertures can be defined by the curved frame 3710 such that oral fluids and air can pass through the holes or apertures and a suction catheter can be administered through one of the holes or apertures on the curved frame 3710.

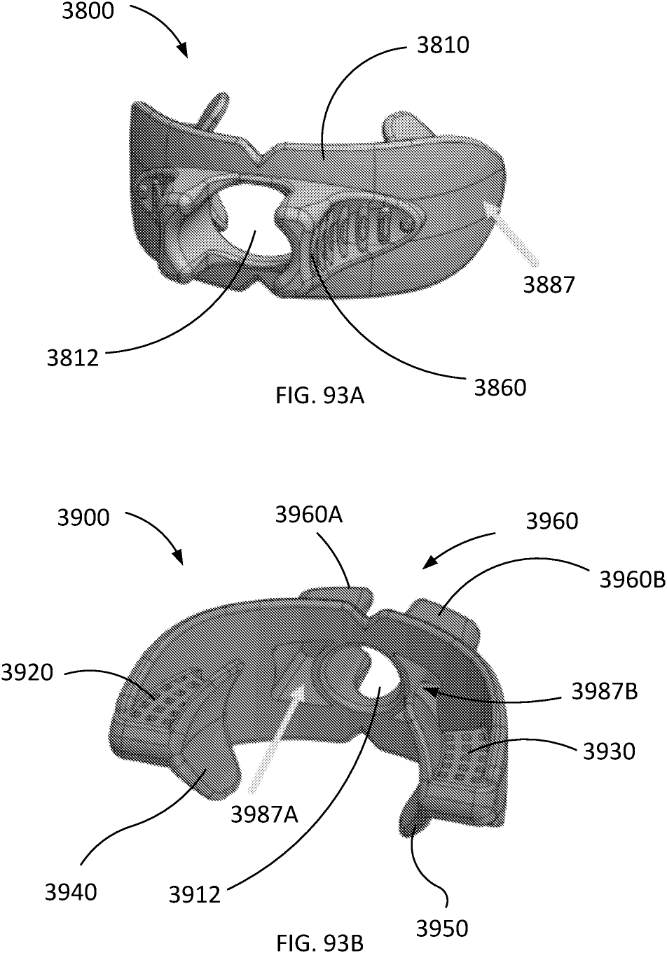

FIG. 93A is a front perspective view of an oral device 3800. The oral device 3800 can be the same or similar in structure and/or function to any of the oral devices described herein, such as the oral device 300 and/or oral device 900. For example, the oral device 3800 includes a curved frame 3810, a handle 3860, and a distal opening 3812. The oral device 3800 includes a dissolvable membrane 3887 that retains a medicament (e.g., embedded in the dissolvable membrane or within a pocket defined by the oral device 3800 and closed by the dissolvable membrane), such that when the dissolvable membrane 3887 dissolves, the medicament can be administered to a user. In some embodiments, the medicament retained by the dissolvable membrane 3887 can be in the form of a gel, a gel cap, a pill, an aqueous solution, powdered drug, and/or any other suitable formulation. The dissolvable membrane 3887 can be configured to dissolve when the dissolvable membrane 3887 engages with the user’s saliva (e.g., when the oral device 3800 becomes disposed within the user’s mouth) or any other suitable liquid. The dissolvable membrane 3887 retaining the medicament can be disposed in any suitable place within the oral device 3800 such that the user’s saliva can engage the dissolvable membrane 3887. In some embodiments, the dissolvable membrane 3887 and the retained medicament can be disposed on the proximal surface of the curved frame 3810 such that the dissolvable membrane 3887 can engage the user’s buccal space (i.e., inner cheeks) when the oral device 3800 is disposed in the user’s mouth. In use, the oral device 3800 can retain a medicament that can be administered to a user when the oral device 3800 is disposed within the user’s mouth. In some implementations, if a user cannot generate a sufficient amount of saliva to dissolve the dissolvable membrane 3887, a user or caregiver can dispense a liquid such as water to the user’s mouth to activate the dissolving of the dissolvable membrane 3887.

FIG. 93B is a rear perspective view of an oral device 3900. The oral device 3900 can be the same or similar in structure and/or function to any of the oral devices described herein, such as the oral device 300, oral device 900, and/or oral device 3800. For example, the oral device 3900 includes a curved frame 3910, a handle 3960, a distal opening 3912, a first bite block 3920, a second bite block 3930, a first wing 3940, and a second wing 3950. The oral device 3900 can define one or more pockets or cavities each sealed by a dissolvable membrane configured to retain medicament within the pocket until dissolution of the membrane (e.g., by a fluid such as saliva). In some embodiments, the handle 3960 can define one or more pockets closed by a dissolvable membrane. For example, the handle 3960 can define a cavity within the handle 3960 such that distal end of the handle 3960 is closed and the proximal end of the handle 3960 coupled to the curved frame 3910 is closed by a dissolvable membrane. In some embodiments, as shown in FIG. 93B, the handle 3960 can include a first handle portion 3960A and a second handle portion 3960B such that the first handle portion 3960A defines a first cavity 3987A and the second handle portion 3960B defines a second cavity 3987B. Each of the first cavity 3987A and the second cavity 3987B can be sealed by a dissolvable membrane.

When the dissolvable membrane(s) of the oral device 3900 dissolves, the medicament can be administered to a user. In some embodiments, the medicament retained by the dissolvable membrane can be in the form of a gel, a gel cap, a pill, an aqueous solution, powdered drug, and/or any other suitable formulation. Similar to oral device 3800, the dissolvable membrane can be configured to dissolve when the dissolvable membrane engages with the user’s saliva (e.g., when the oral device 3900 becomes disposed within the user’s mouth) or any other suitable liquid. In use, the oral device 3900 can administer medicament retained by the first dissolvable membrane and second dissolvable membrane when the oral device 3900 is disposed within the user’s mouth and the user’s saliva dissolves the first dissolvable membrane and/or second dissolvable membrane. In some implementations, if a user cannot generate a sufficient amount of saliva to dissolve the first dissolvable membrane or second dissolvable membrane, a user or caregiver can dispense a liquid such as water to the user’s mouth to activate the dissolving of the first dissolvable membrane and/or second dissolvable membrane.

In some embodiments, at least a portion of oral device 3800 or oral device 3900 can be formed of a material that contains medicament and is dissolvable in saliva. For example, the oral device 3800 or oral device 3900 can be formed from food grade material (e.g., gelatin) that includes a medicament, and which material dissolves upon contact with saliva such that the oral device 3800 or oral device 3900 can administer the medicament when the oral device 3800 or oral device 3900 is disposed in the user’s mouth and in contact with the user’s saliva. Thus, a dissolvable oral device can be intended for a single-use.

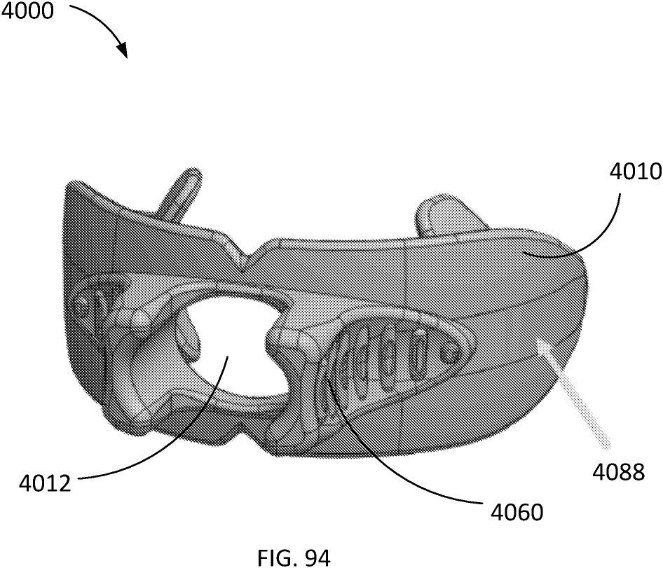

FIG. 94 is a front perspective view of an oral device 4000. The oral device 4000 can be the same or similar in structure and/or function to any of the oral devices described herein, such as the oral device 300 and/or oral device 3800. For example, the oral device 4000 includes a curved frame 4010, a handle 4060, and a distal opening 4012. The oral device 4000 includes at least one pocket (not shown, but located, for example, at location 4088). The pocket can be formed, for example, as a blind hole. The pocket can be configured to retain a dissolvable medicament such as a drug cake or a chewable tablet. The medicament can be configured to dissolve when the medicament engages a liquid, such as a user’s saliva. Thus, when the oral device 4000 is disposed in the user’s mouth and the medicament in the pocket is in contact with the user’s saliva, the medicament can dissolve and be administered to the user (e.g., over a period of time while the user is wearing the device). The pocket retaining the medicament can be defined in any suitable place within the oral device 4000 such that the user’s saliva can contact the dissolvable medicament within the pocket. For example, the pocket can be defined by the curved frame 4010. In some embodiments, the pocket can be disposed on a portion of the curved frame 4010 such that the medicament within the pocket can face and/or contact (prior to dissolution) the user’s buccal areas (i.e., inner cheeks). In some embodiments, the medicament can be friction fit into the pocket. In some implementations, if a user cannot generate a sufficient amount of saliva to dissolve the dissolvable medicament within the pocket 4088, a user or caregiver can dispense a liquid such as water into the user’s mouth to activate the dissolving of the dissolvable medicament.

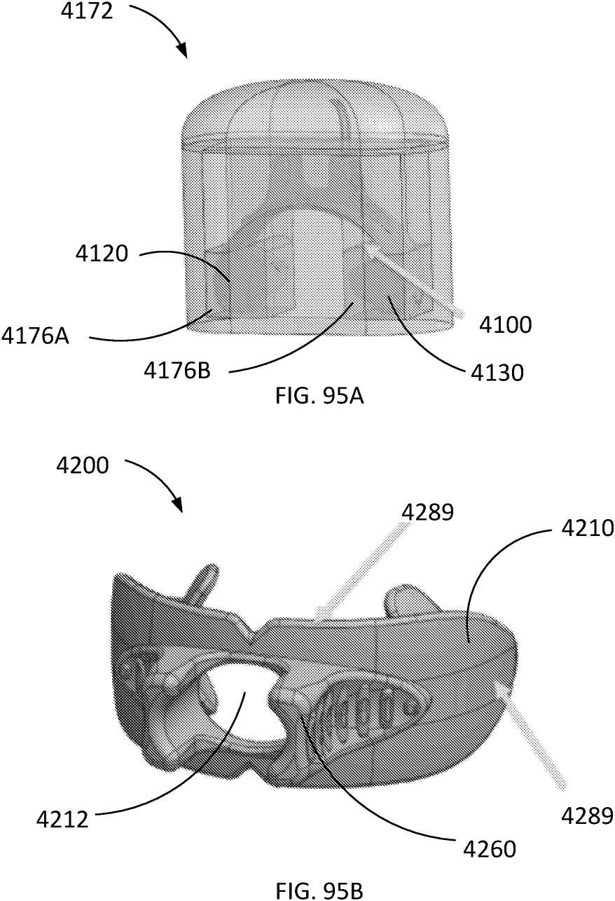

FIG. 95A illustrates an oral device 4100 disposed within a storage container 4172. The storage container 4172 can include a body and a lid that is removable from or rotatable relative to (e.g., via a hinge) the body such that the oral device 4100 can be removed from an interior of the storage container 4172. The oral device can be the same or similar in structure and/or function to any of the oral devices described herein, such as the oral device 300 and/or oral device 3500. For example, the oral device 4100 includes a first bite block 4120 and a second bite block 4130. The interior of the storage container 4172 can include a first bath 4176A (also referred to as a first reservoir) and a second bath 4176B (also referred to as a second reservoir). When the oral device 4100 is disposed within the storage container 4172, the first bath 4176A can receive the first bite block 4120 and the second bath 4176B can receive the second bite block 4130. The first bath 4176A and second bath 4176B can contain a liquid or aqueous medicament, such that when the first bite block 4120 is removed from the first bath 4176A, a portion of medicament can be retained within or upon the first bite block 4120 and when the second bite block 4130 is removed from the second bath 4176B, a portion of medicament can be retained within or upon the second bite block 4130. Thus, when a user or caregiver removes the oral device 4100 from storage container 4172, the oral device 4100 includes a medicament disposed within or upon the first bite block 4120 and/or second bite block 4130. In some embodiments, the first bite block 4120 and/or second bite block 4130 can be shaped such that the surface and/or cavities of the first bite block 4120 and the second bite block 4120 can retain medicament. For example, the first bite block 4120 and/or second bite block 4130 can include or define a set of ridge portions, and/or a set of projections or bumps to increase the surface area for retaining medicament. In some embodiments, the first bite block 4120 and/or second bite block 4130 can be formed of a sponge-like material or as a waffle-like structure, can define apertures or through-holes, can define partial through-holes (e.g., blind holes), and/or any other suitable cavities to retain medicament such that when the oral device 4100 is disposed in a user’s mouth and the user bites down upon the first bite block 4120 and/or second bite block 4130, the medicament is released from within the first bite block 4120 and/or second bite block 4130 to be administered to the user. In some embodiments, rather than including two separate baths to each receive one of the first bite block 4120 and the second bite block 4130, the storage container 4172 can define one reservoir configured to contain liquid medicament and to receive both of the first bite block 4120 and the second bite block 4130. In some embodiments, the storage container 4172 can be formed of a rigid material, such as a rigid plastic. In some embodiments, the storage container 4172 can be formed of a pouch defining a reservoir and configured to contain the oral device 4100 such that at least the first bite block 4120 and the second bite block 4130 are disposed in a liquid medicament solution contained within the reservoir.

FIG. 95B is a front perspective view of an oral device 4200. The oral device 4200 can be the same or similar in structure and/or function to any of the oral devices described herein, such as the oral device 600 and/or oral device 700. For example, the oral device 4200 includes a curved frame 4210, a handle 4260, and a distal opening 4212. The oral device 4200 includes a powdered medicament (not shown) coated on portions of the oral device 4200. For example, the oral device 4200 includes powdered medicament on the distal surfaces 4289 of the curved frame 4210 such that the powdered medicament can engage with the user’s buccal areas (i.e., inner cheeks and/or inner lips). In some embodiments, the powdered medicament can be dissolvable with a liquid, such as saliva from the user. In use, the oral device 4200 can retain a powdered medicament 4289 that can be administered to a user when the oral device 4200 is disposed within the user’s mouth.

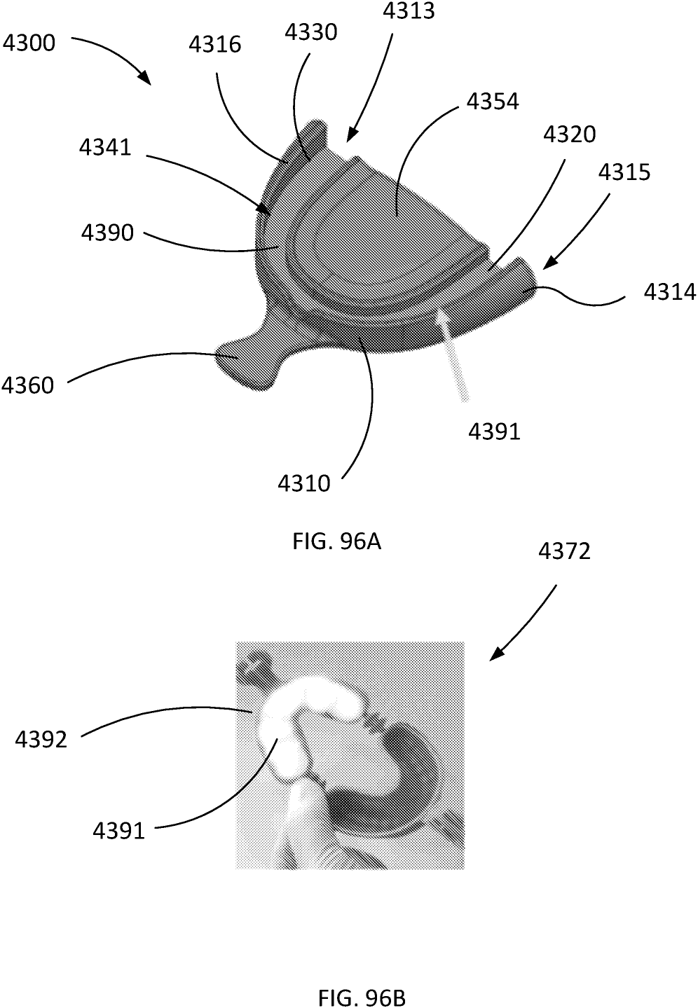

FIGS. 96A and 96B are views of an oral device 4300. The oral device 4300 can be the same or similar in structure and/or function to any of the oral devices described herein, such as the oral device 700 . For example, the oral device 4300 includes a curved frame 4310 having a first end 4313 and a second end 4315, a first cheek protector portion 4314, a second cheek protector portion 4316, a first bite block portion 4320, a second bite block portion 4330, a handle 4360 extending distally from a curved frame 4310, and a lingual frame 4354, and can define a channel 4341 from the first end 4313 to the second end 4315 of the curved frame. Similar to oral device 700, the curved frame 4310, the first bite block 4320, and the second bite block 4330, can be monolithically formed as one continuous curved structure. The channel 4341 can be configured to receive the upper teeth or the lower teeth of a user. In some embodiments, a retention material 4390, such as a wax material, can be disposed within the channel 4341. Similar to oral device 700, the retention material can be configured to adhere to the upper or lower teeth of the user to secure the oral device 4300 in place relative to the user’s upper or lower teeth, respectively. The oral device 4300 can also include a medicament 4391 (e.g., a paste medicament) disposed in the channel 4341 on the retention material 4390. The paste medicament 4391 can be administered to a user by inserting the oral device 4300 in the mouth of the user and engaging the user’s upper teeth or lower teeth with the retention material 4390. When the oral device 4300 is disposed in the user’s mouth and engaged with the user’s teeth, the paste medicament 4391 can flow into the user’s teeth and gums and/or be swallowed by the user. In some embodiments, the paste medicament 4391 has a gel or paste-like consistency that permits the paste medicament 4391 to be retained in the channel 4341 until the user engages the paste medicament 4391 with their upper teeth. In some embodiments, the oral device 4300 can be stored within a storage container 4372. In some embodiments, the storage container 4372 includes a cover 4392 to protect the retaining material 4390, and can be made of any suitable material, such as plastic. In some embodiments, the cover 4392 can include paste medicament 4391 such that when the cover 4392 is disposed on the retaining material 4390, the paste medicament 4391 can be transferred from the cover 4392 to the retaining material 4390 of the oral device 4300.

As discussed herein, an oral device (such as the oral devices 3100 through 4100) can include and/or contain a medicament to be administered to a user. In some embodiments, any of the medicaments and administration mechanisms described herein can be combined to administer one or more medicaments to a user. In some embodiments, a medicament included in or contained by an oral device such as any of the oral devices described herein can also have additional properties that can aid the administration of the medicament. For example, an oral device can include or contain a catalyst that can activate an administration mechanism of the oral device as a result of a chemical reaction between the catalyst and the user’s saliva. In some embodiments, for example, the catalyst can be activated to expand to force open a valve or other mechanical release to administer a medicament retained by the valve. In some embodiments, the catalyst can be configured to activate for or after a predetermined time period (e.g., the catalyst can activate the administration mechanism for 5 minutes after exposure to saliva or 5 minutes after exposure to saliva). In some embodiments, an oral device and/or medicament can be activated by body heat. For example, an oral device can be packaged in a dormant, unactivated state. When the oral device is disposed in a user’s mouth, body heat from the user’s mouth can activate the mechanism to administer the medicament. For example, a medicament retaining membrane can retain medicament within a cavity of the oral device and can be formed of a material that deforms in response to a temperature of the membrane being raised above a threshold temperature (e.g., a temperature at or near the temperature of the user’s body or mouth). In some embodiments, an oral device can include a pressure mechanism to administer medicament. For example, an elastomeric pump can be disposed within an oral device between a first biting surface and a second biting surface opposite the first biting surface of the oral device, such that when a user compresses the first biting surface toward the second biting surface (e.g., via biting the first biting surface and the second biting surface), the elastomeric pump can apply sufficient pressure to a valve or membrane of the oral device (e.g., via increasing the pressure of an interior chamber including medicament) to open the valve or break the membrane such that the medicament is dispensed to the user. In some embodiments, the elastomeric pump can be configured to increase the pressure within the interior chamber incrementally with each compression (e.g., bite) of the user on the elastomeric pump. In some embodiments, the elastomeric pump can draw air into the pump between compressions and push air from the pump during each compression.

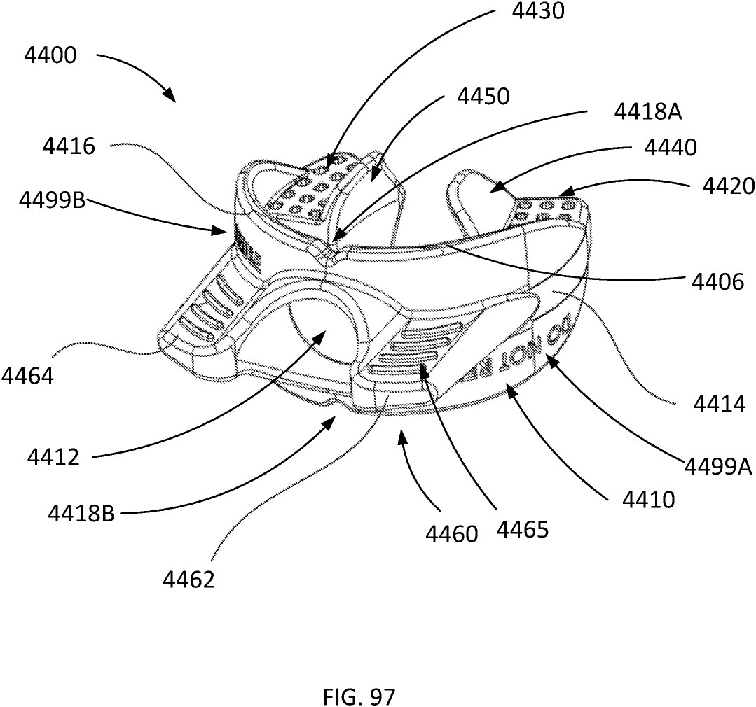

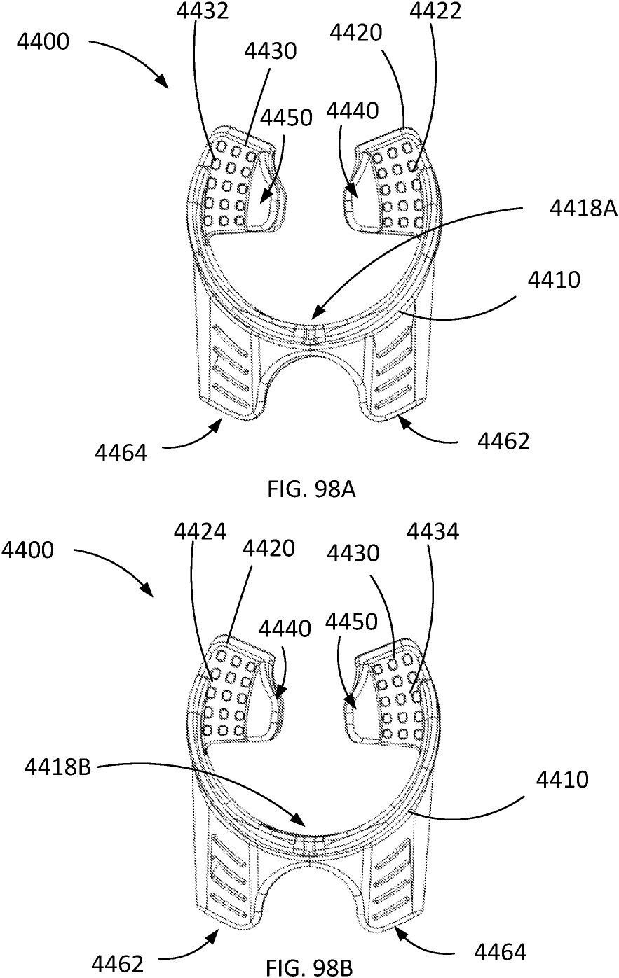

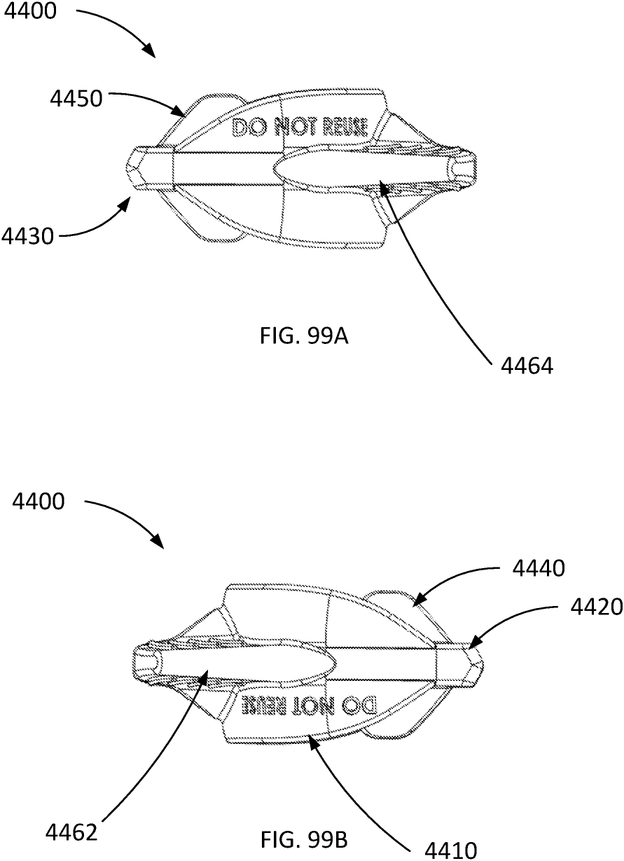

FIGS. 97-100B are front perspective, top, bottom, first side, second side, front, and rear views, respectively, of an oral device 4400. The oral device 4400 can be the same or similar in structure and/or function to any of the oral devices described herein, such as the oral device 300 and/or the oral device 1000. For example, the oral device 4400 includes a curved frame 4410, a handle 4460 having a first handle portion 4462 and a second handle portion 4464, a first notch 4418A, a second notch 4418B, and grip portions 4465, which may be the same or similar in structure and/or function to similarly named components of the oral device 300 and/or the oral device 1000. Additionally, the frame 4412 defines a distal opening 4412. As shown in FIG. 97, the oral device 4400 also includes a first bite block 4420, a second bite block 4430, a first wing 4440, and a second wing 4450 that may be the same or similar in structure and/or function to similarly named components of the oral device 300 and/or the oral device 1000. As shown in FIG. 97, the first handle portion 4462 and the second handle portion 4464 extend separately from the front surface of the curved frame 4410 from either side of a concave portion surrounding the opening 4412. Additionally, as shown in FIGS. 99A and 99B, the first bite block 4420 and the second bite block 4440 can extend farther proximally than cheek protector portions of the curved frame 4410, which taper toward the width of the first bite block 4420 and the second bite block 4430.

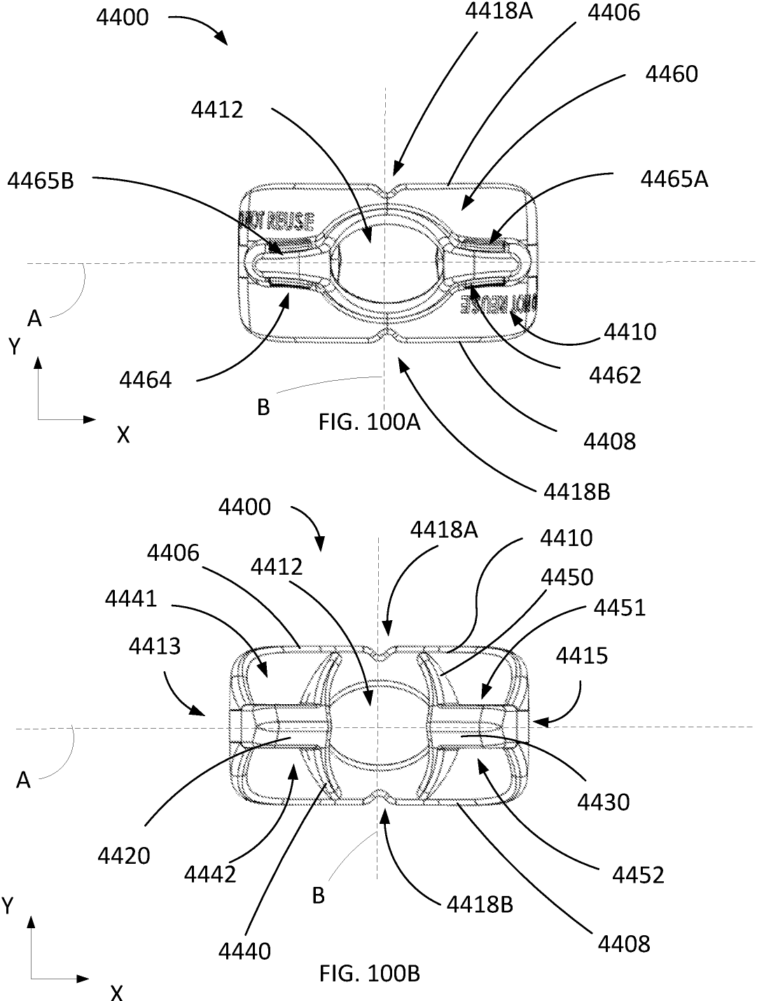

The curved frame 4410 can include or define indications 4499A and 4499B. The indications 4499A and 4499B can include, for example, a product label, instructions, and/or a warning for the user and/or caregiver. For example, as shown in FIGS. 100A and 100B, the indications 4499A and 4499B can include a “DO NOT REUSE” message to indicate to the user and/or a caregiver that each oral device 4400 is intended for one time use only. As shown in FIG. 100A, the indications 4499A and 4499B can each be oriented to be read upright relative to a plane A (described in more detail below).

As shown in FIGS. 100A and 100B, with the exception of indications 4499A and 4499B, the oral device 4400 is symmetrical about a plane A including a portion of the frame 4410, a portion of the first bite block 4420, a portion of the second bite block 4430, a portion of the first wing 4440, a portion of the second wing 4450, and a portion of the handle 4460. The plane A can be an X-Z plane (the Z axis extending into and out of the page), as shown in FIGS. 100A and 100B. The oral device 4400 is also symmetrical about a plane B (with the exception of indications 4499A and 4499B), which can be a Y-Z plane as shown in FIGS. 100A and 100B. Thus, the oral device 4400 can be inserted into the mouth of the user in one of two orientations. For example, the oral device 4400 can be inserted such that the first bite block 4420 is configured to be engaged by the left teeth of the user and the second bite block 4430 is configured to be engaged by the right teeth of the user, and the oral device can be inserted such that the first bite block 4420 is configured to be engaged by the right teeth of the user and the second bite block 4430 is configured to be engaged by the left teeth of the user, eliminating the need for a user or a caregiver to determine the proper orientation of the oral device 4400 before insertion into the user’s mouth.

The first handle portion 4462 and the second handle portion 4464 can each include an upper or first planar surface and a lower or second planar surface opposite the upper or first planar surface. Each of the first handle portion 4462 and the second handle portion 4464 extend away from the opening 4412 (e.g., along plane A). Each of the first handle portion 4462 and the second handle portion 4464 can extend away from the distal surface of the curved frame 4410 a sufficient distance such that distal portions of the first handle portion 4462 and the second handle portion 4464 can be grasped by fingers of the user or caregiver (e.g., an index finger and a thumb) when the oral device 4400 is disposed in the user’s mouth and the lips of the user are contacting or near proximal portions of the first handle portion 4462 and the second handle portion 4464.

In some embodiments, the first handle portion 4462 and the second handle portion 4464 are configured to be urged toward each other to cause the frame 4410 to expand from a first configuration in which the first end 4413 and the second end 4415 of the frame 4410 are a first distance apart to a second configuration in which the first end 4413 and the second end 4415 of the frame 4410 are a second distance apart greater than the first distance. Thus, the oral device 4400 can be transitioned from the first configuration to the second configuration for insertion into the user’s mouth, and then allowed to transition from the second configuration to the first configuration when within the user’s mouth for engagement (e.g., lateral engagement) with the user’s teeth. In some embodiments, the oral device 4400 can be biased toward the first configuration. As shown in FIG. 97, the first handle portion 4462 and the second handle portion 4464 can each include a plurality of grip portions (e.g., raised portions or ridges) disposed along the first handle portion 4462 and the second handle portion 4464 to assist the user in gripping the handle 4460. The first notch 4418A and the second notch 4418B can assist the frame 4410 in transitioning from the first configuration to the second configuration due to the frame 4410 expanding (e.g., hinging) about an axis extending through the first notch 4418A and the second notch 4418B.

In some embodiments, rather than the first handle portion 4462 being contractable toward the second handle portion 4464 to cause the oral device 4400 to expand from the first configuration to the second configuration, the oral device 4400 can be biased toward the first configuration and can be transitioned to the second configuration as the oral device 4400 is pushed into a user’s mouth by the user’s teeth. Specifically, the ends of the curved frame 4410 can contact the sides of the user’s teeth and be urged outward by the user’s teeth as the oral device 4400 is pushed proximally. In some embodiments, the first bite block 4420 and the second bite block 4430 can be closer together in the first configuration than when the oral device 4400 is disposed in the user’s mouth and engaged with the user’s teeth (e.g., in the second configuration). In some embodiments, the first handle portion 4462 and the second handle portion 4464 are rigid such that the handle portions cannot be urged toward one another. In some embodiments, the first handle portion 4462 and the second handle portion 4464 can be monolithically formed with the curved frame 4410 and wings 4440, 4450, but cannot be moved (e.g., urged) relative to the portions of the curved frame 4410 to which the first handle portion 4462 and the second handle portion 4464 are attached.

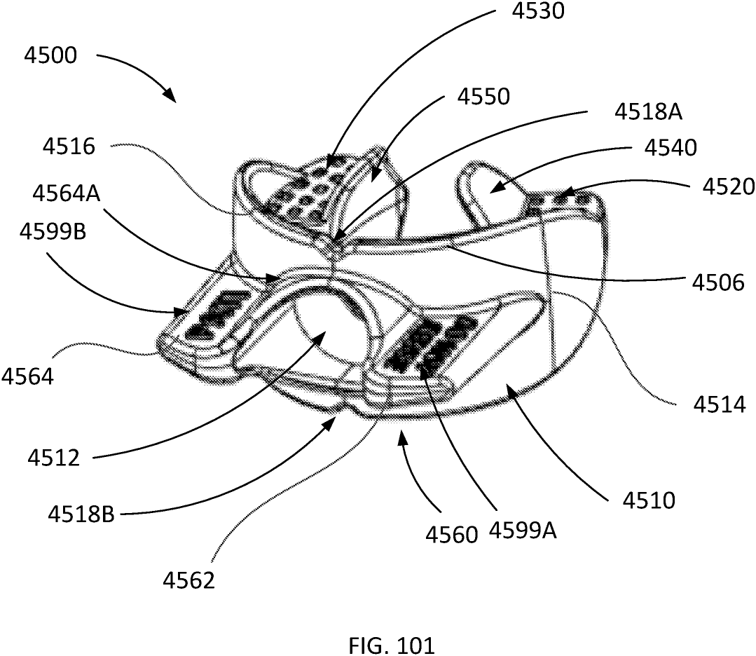

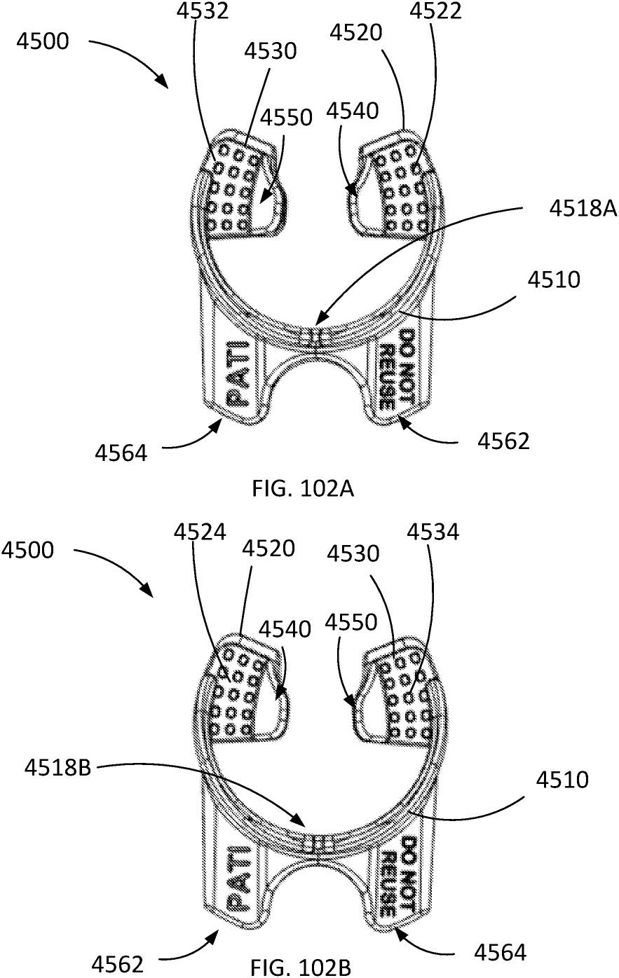

FIGS. 101-102B are front perspective, top, and bottom views, respectively, of an oral device 4500. In some embodiments, a back view of the oral device 4500 may look the same or similar to the back view of the oral device 4400 shown in FIG. 100B. The oral device 4500 can be the same or similar in structure and/or function to any of the oral devices described herein, such as the oral device 100, the oral device 300, the oral device 1000 and/or the oral device 4400. For example, the oral device 4500 includes a curved frame 4510, a handle 4560 having a first handle portion 4562 and a second handle portion 4564, an optional first notch 4518A, and an optional second notch 4518B, which may be the same or similar in structure and/or function to similarly named components of the oral device 100, the oral device 300, the oral device 1000 and/or the oral device 4400. Additionally, the frame 4512 defines a distal opening 4512. Similar to the oral device 4400, the first handle portion 4562 and the second handle portion 4564 can each be tab-shaped, and can be coupled to each other via a tubular central handle portion 4564A that defines an opening aligned with the distal opening 4512. The tubular central handle portion 4564A can define a recess (e.g., a curved or U-shaped recess) in a top and bottom surface of the tubular central handle portion 4564A (e.g., vertically above and below the first handle portion 4562 and the second handle portion 4564) for easier physical and visual access to the distal opening 4512, and such that the range of movement of a catheter within the opening (e.g., a vertical range of motion) is increased compared to a tubular opening without a recess. The vertical range of motion can be larger than the horizontal or lateral range of motion. As shown in FIG. 101, the oral device 4500 also includes a first bite block 4520, a second bite block 4530, a first wing 4540, and a second wing 4550 that may be the same or similar in structure and/or function to similarly named components of the oral device 100, the oral device 300, the oral device 1000 and/or the oral device 4400. As shown in FIG. 101, the first handle portion 4562 and the second handle portion 4564 extend separately from the front surface of the curved frame 4510 from either side of a concave portion surrounding the opening 4512.

Each of the first handle portion 4562 and the second handle portion 4564 can include or define indications 4599A and 4599B. The indications 4599A and 4599B can include, for example, a product label, instructions, and/or a warning for the user and/or caregiver. For example, as shown in FIG. 101, the indications 4599A and/or 4599B can include a “DO NOT REUSE” message to indicate to the user and/or a caregiver that each oral device 4500 is intended for one time use only and/or a message indicating the name of the device (e.g., “PATI”) such that it is easily located and/or identified by the user, a caregiver, or a stranger (e.g., in an emergency situation in which a bracelet, necklace, or wallet card of the user indicates the user needs to be administered the named device). The indications 4599A and 4599B can be raised relative to a surface of the first handle portion 4562 or the second handle portion, respectively, such that the indications 4599A and 4599B can be used to improve the grip of a user and/or caregiver grasping the handle 4560. As shown in FIGS. 102A and 102B, the first handle portion 4562 and the second handle portion 4564 can each include an upper or first planar surface and a lower or second planar surface opposite the upper or first planar surface, with an indication on each surface.

In some embodiments, the first handle portion 4562 and the second handle portion 4564 are rigid such that the handle portions cannot be urged toward one another. In some embodiments, the first handle portion 4562 and the second handle portion 4564 can be monolithically formed with the curved frame 4510 and wings 4540, 4550, but cannot be moved (e.g., urged) relative to the portions of the curved frame 4510 to which the first handle portion 4562 and the second handle portion 4564 are attached. In some embodiments, the oral device 4500 is packaged in a contracted configuration in which a first end of the curved frame 4510 and a second end of the curved frame 4510 are a first distance apart (and the first bite block 4520 and the second bite block 4530 are a first distance apart). The oral device 4500 can be biased toward the contracted configuration. The oral device 4500 can transition to an expanded configuration as the oral device 4500 is pushed into a user’s mouth by the user’s teeth. Specifically, the ends of the curved frame 4510 can contact the sides of the user’s teeth and be urged outward by the user’s teeth as the oral device 4500 is pushed proximally. The ends of the curved frame (and the first bite block 4520 and the second bite block 4530) can be farther apart in the expanded configuration than in the contracted configuration.

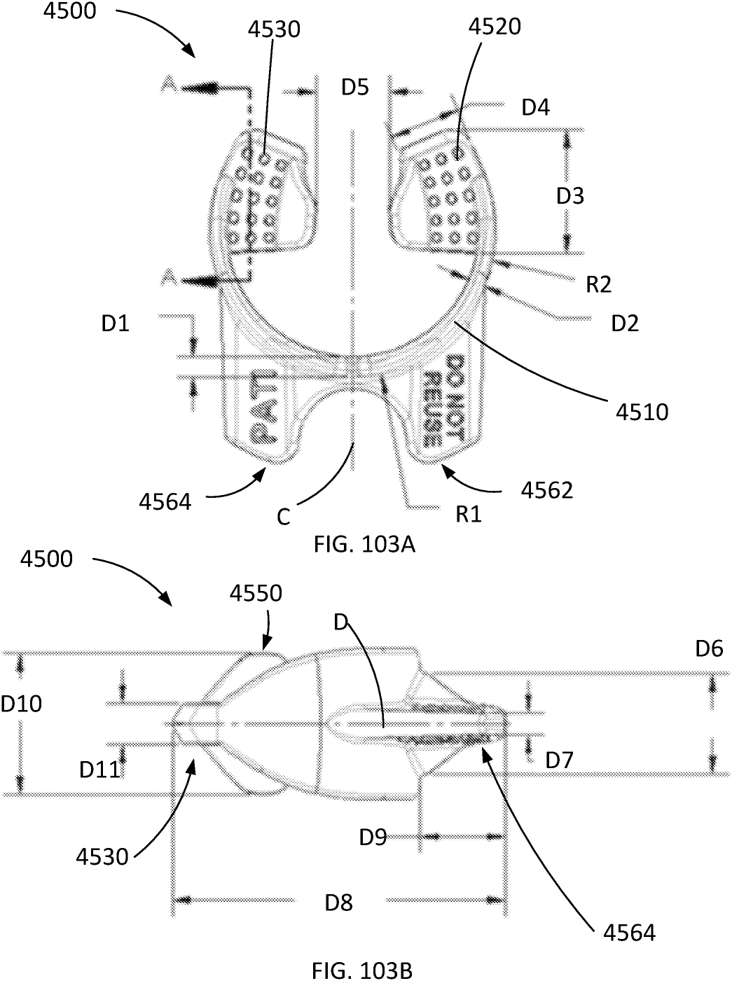

The oral device 4500 can include any suitable dimensions to engage with a user’s mouth. For example, as shown in FIG. 103A, which can be a top or bottom view of the oral device 4500 (e.g., in an unflexed initial configuration prior to insertion into a user’s mouth), the curved frame 4510 can have a thickness D1 along a central axis C of the curved frame 4510, and a thickness D2 towards the first end of the curved frame 4510. The thickness D1 can be, for example, about 3.9 mm and the thickness D2 can be, for example, about 3.1 mm. The curved frame 4510 can have a radius of curvature R2 in a first contracted configuration toward which the oral device 4500 is biased. The radius of curvature R2 can be, for example, about 26.4 mm. The first bite block 4520 can have a length D3 from a distalmost portion of the first bite block 4520 to a proximal-most portion of the first bite block 4520. The length D3 can be, for example, about 22.8 mm. The first bite block 4520 can have a width D4 from an innermost edge to an opposing outer edge along a proximal surface of the first bite block 4520. The width D4 can be, for example, about 13.1 mm. The first bite block 4520 and the second bite block 4530 can have the same dimensions. The first wing 4540 can be separated from the second wing 4550 by a distance D5 in the first contracted configuration. The distance D5 can be, for example, about 13.5 mm.

As shown in FIG. 103B, which can be a first (e.g., left) side view or a second (e.g., right) side view of the oral device 4500, the handle 4560 has a height D6 from a bottommost portion of the handle 4560 to an uppermost portion of the handle 4560 (e.g., the height of the concave portion defining the opening 4512). The height D6 can be, for example, about 14.1 mm. Each of the first handle portion 4562 and the second handle portion 4564 can have a height D7. The height D7 can be, for example, about 3.9 mm. The oral device 4400 can have a length D8 from a distal end to a proximal end of the oral device 4400 (e.g., taken along center line D). The length D8 can be, for example, about 61.6 mm. The handle 4560 extends a distance D9 beyond the front surface of the curved frame 4510 along center line D. The distance D9 can be, for example, about 15.7 mm. In some embodiments, the distance D9 can be, for example, between about 1 cm and about 2 cm, between about 17 mm and about 20 mm, between about about 20 mm and about 25 mm, between about 25 mm and about 30 mm, between about 30 mm and about 35 mm, greater than 35 mm, or any other suitable distance. The oral device 4500 and/or the first wing 4540 and the second wing 4550 can have a height D10. The height D10 can be, for example, about 26 mm. The first bite block 4520 and the second bite block 4530 can each have a height D11. The height D11 can be, for example, about 7.5 mm.

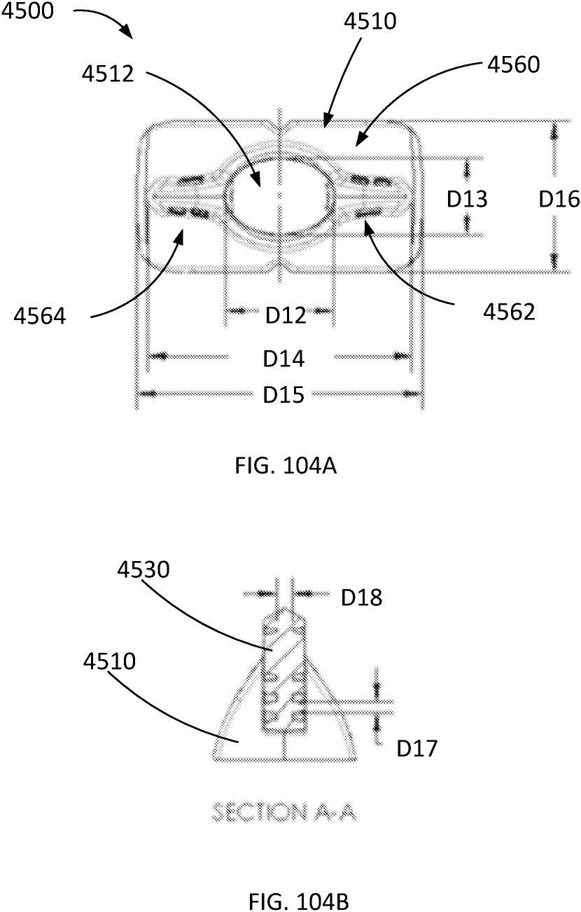

As shown in FIG. 104A, which is a front view of the oral device 4500, the distal opening 4512 can have a width D12 and a height D13. The width D12 can be, for example, about 20 mm, and the height D13 can be, for example, 14.1 mm. The handle 4560 can have an overall width D14. The width D14 can be, for example, 49 mm. The oral device 4500 and the curved frame 4510 can have an overall width D15 and height D16. The overall width D15 can be, for example, about 52.8 mm. The overall height D16 can be, for example, about 28 mm. In some embodiments, the height D16 and the height D10 can be about the same (e.g., within one, two, three, or four millimeters). As shown in FIG. 104B, which is a cross-section of the oral device 4500 taken along line A-A in FIG. 103A, the second bite block 4530 can define cavities spaced by a distance D17 along each surface of the second bite block 4530. The distance D17 can be, for example, about 2 mm. Each cavity on a first side of the second bite block 4530 can be spaced a distance D18 from an opposing cavity (e.g., a coaxial cavity) on a second side of the second bite block 4530. The distance D18 can be, for example, about 3 mm. It should be understood that the aforementioned dimensions are an example of an oral device 4500 that conforms to the mouth size of most adults. The dimensions may vary slightly based on the patient age demographic, i.e. pediatric, pre-adolescent and adolescent that conform to the mouth size of most people within a certain age demographic. In some embodiments, some or all of relative dimensions may maintain the same ratio when the oral device 4500 is sized for a larger or smaller mouth. For example, the ratio of distance D10 to distance D16, the ratio of distance D10 to distance D11, the ratio of distance D3 to distance D4, the ratio of distance D4 to distance D11, the ratio of distance D12 to distance D13, the ratio of distance D16 to distance D13, the ratio of distance D15 to D16, the ratio of distance D14 to D13, and/or any other ratios between any other distances described with respect to the oral device 4500 can be maintained. In some embodiments, one or more of the distances described with respect to the oral device 4500 can be increased or decreased. For example, the distance D9 can be increased relative to the other distances for better gripping of the handle 4560, but not increased so much that the handle 4560 may be likely to be bumped into something and accidentally dislodged during a seizure event.

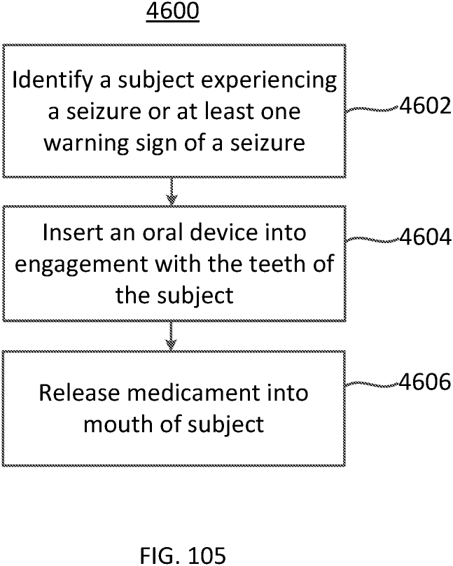

FIG. 105 is a flow chart illustrating a method 4600. The method 4600 can be the same or similar to any of the method described herein, such as the method 200 described above. As shown in FIG. 105, the method 4600 includes identifying 4602 a subject experiencing a seizure or at least one warning sign of a seizure. In some embodiments, the identifying can be performed by the subject. In some embodiments, the identifying can be performed by a person other than the subject (e.g., a caregiver, medical practitioner, or a stranger). An oral device can be inserted 4604 into engagement with the teeth of the subject. In some embodiments, the oral device can be the same or similar in structure and/or function to any of the oral devices described herein, such as any of the oral devices configured to dispense medicament disclosed herein. A medicament can be released 4606 into the mouth of the subject (e.g., via any of the administration methods described with respect to the oral devices described herein). In some embodiments, a distal end of a catheter (e.g., a suction catheter), also referred to as a tubular member, can be inserted through an opening (e.g., a central distal opening) in the oral device into a portion of the user’s mouth proximal of the user’s front teeth. In some embodiments, the catheter can be inserted into the user’s mouth during a tonic phase of a tonic-clonic seizure. The catheter can be operated to draw fluid out of the user’s mouth or administer medication into a user’s mouth.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components and/or features of the different embodiments described.

1. An oral device, comprising:
a curved frame having a first end and a second end;
a first bite block coupled to the first end of the curved frame;
a second bite block coupled to the second end of the curved frame;
a first wing coupled to the first bite block;
a second wing coupled to the second bite block, the first wing and the second wing configured to corral a tongue of a user such that the tongue of the user is restrained from contacting a tooth engaging surface of the left bite block or the right bite block when the oral device is disposed in a mouth of the user, the oral device being symmetrical about a plane including a portion of the curved frame, a portion of the first bite block, and a portion of the second bite block; and
a handle coupled to and extending distally from a distal surface of the curved frame a sufficient distance such that, when the oral device is disposed in the user’s mouth, a distal portion of the handle extends from the user’s mouth and can be grasped by the user.

2. The oral device of claim 1, wherein the handle includes a first handle portion and a second handle portion, each of the first handle portion and the second portion coupled to and extending from a distal surface of the curved frame, the handle defining a space between the first handle portion and the second handle portion aligned with an opening in the curved frame.

3. The oral device of claim 1, wherein the oral device does not include any portion contacting a non-lip surface of a face of the user when the oral device is disposed in the mouth of the user.

4. The oral device of claim 1, wherein:
the first bite block includes a first tooth engaging surface configured to contact a plurality of upper teeth of the user and a second tooth engaging surface configured to contact a plurality of bottom teeth of the user, the first tooth engaging surface of the first bite block being parallel to the second tooth engaging surface of the first bite block, and
the second bite block includes a first tooth engaging surface configured to contact a plurality of upper teeth of the user and a second tooth engaging surface configured to contact a plurality of bottom teeth of the user, the first tooth engaging surface of the second bite block being parallel to the second tooth engaging surface of the second bite block.

5. The oral device of claim 4, wherein the first bite block is sufficiently thick between the first tooth engaging surface of the first bite block and the second tooth engaging surface of the first bite block and the second bite block is sufficiently thick between the first tooth engaging surface of the second bite block and the second tooth engaging surface of the second bite block such that, when the oral device is disposed in the mouth of the user, the user is prevented from simultaneously applying a biting force to the tongue of the user with an upper front tooth and a lower front tooth to damage the tongue.

6. The oral device of claim 1, wherein the first bite block is coupled to the second bite block only via the curved frame.

7. The oral device of claim 1, wherein, when the oral device is disposed in the mouth of the user and the first wing and the second wing are corralling a tongue of a user, the tongue of the user is able to contact a roof of the mouth and a floor of the mouth.

8. The oral device of claim 1, wherein the first wing extends from a distal edge to a proximal edge of the first bite block and the second wing extends from a distal edge to a proximal edge of the second bite block.

9. The oral device of claim 1, wherein the first handle portion and the second handle portion are configured to be urged toward each other to transition the curved frame from a first configuration in which the first end and the second end of the curved frame are a first distance apart to a second configuration in which the first end and the second end of the curved frame are a second distance apart greater than the first distance.

10. The oral device of claim 1, wherein the first wing is curved away from the first bite block and the second wing is curved away from the second bite block.

11. The oral device of claim 1, wherein the first wing and the second wing each have an overall height that is about the same as the maximum height of the curved frame.

12. The oral device of claim 1, wherein the first wing and the second wing each have an overall height that is at least about twice the height of the first bite block and the second bite block, respectively.

13. The oral device of claim 1, wherein the first wing and the second wing each have a sufficient overall height such that, when the oral device is disposed in the mouth of the user, the first wing and the second wing each contact at least one of the roof of the mouth or the floor of the mouth.

14. The oral device of claim 1, wherein the first bite block defines a pocket within which medicament can be disposed, the pocket configured to release the medicament in response to the user applying a biting force to the first bite block.

15. The oral device of claim 1, further comprising a receptacle configured to contain medicament, the handle defining a through-hole within which the receptacle can be transitioned between a closed configuration and an open configuration, the receptacle defining an opening via which the medicament can exit the receptacle when the receptacle is in the open configuration relative to the handle.

16. The oral device of claim 1, further comprising a canister coupled to the curved frame and configured to dispense medicament through an opening in the curved frame.

17. The oral device of claim 1, wherein the handle defines a reservoir within which medicament can be disposed, the handle configured to urge the medicament from the reservoir through an opening in the curved frame in response to the handle being squeezed.

18. An oral device, comprising:
a curved frame having a first end and a second end;
a first bite block coupled to the first end of the curved frame;
a second bite block coupled to the second end of the curved frame; and
a handle coupled to and extending distally from the curved frame, the handle having a first handle portion and a second handle portion, the first handle portion configured to be urged toward the second handle portion to transition the curved frame from a first configuration in which the first end and the second end of the curved frame are a first distance apart to a second configuration in which the first end and the second end of the curved frame are a second distance apart greater than the first distance.

19. The oral device of claim 18, wherein a proximal portion of the first handle portion is configured to be urged toward the second handle portion to transition the curved frame to a third configuration in which the first end and the second end of the curved frame are a third distance apart less than the first distance.

20. The oral device of claim 18, wherein the curved frame defines a distal opening configured to provide access to a mouth of a user through the opening when the oral device is disposed in the mouth of the user, the first handle portion disposed on a first side of the opening and the second handle portion disposed on a second side of the opening.

21. The oral device of claim 20, wherein the handle defines a first recess disposed above the opening and a second recess defined below the opening, the first recess and the second recess defined between the first handle portion and the second handle portion.

22. The oral device of claim 20, wherein the opening is configured to allow for airflow and to receive a portion of a catheter such that fluid can be transferred relative to the mouth of the user via the catheter.

23. (canceled)

24. The oral device of claim 18, wherein the frame defines a first notch and a second notch, the first notch and the second notch disposed on opposite edges of the frame in a center portion of the frame.

25. The oral device of claim 18, wherein the curved frame is biased toward the first configuration.

26. (canceled)

27. A method, comprising:
identifying a subject experiencing a seizure or at least one warning sign of a seizure; and
inserting an oral device into engagement with a tongue and teeth of the subject, the oral device including:
a curved frame having a first end and a second end,
a first bite block coupled to the first end of the curved frame,
a second bite block coupled to the second end of the curved frame,
a first wing coupled to the first bite block,
a second wing coupled to the second bite block, the first wing and the second wing configured to corral a tongue of the subject such that the tongue of the subject is restrained from contacting a tooth engaging surface of the left bite block or the right bite block when the oral device is disposed in a mouth of the subject, and
a handle extending distally from the curved frame a sufficient distance such that a distal portion of the handle can be grasped between an index finger and a thumb when the oral device is disposed in the subject’s mouth.

28. The method of claim 27, wherein the inserting is performed by the subject experiencing the seizure or the at least one warning sign of the seizure.

29. The method of claim 27, wherein the inserting is performed by a caregiver of the subject experiencing the seizure or the at least one warning sign of the seizure.

30. The method of claim 27, further comprising inserting a catheter through an opening in the curved frame.

31-35. (canceled)

36. The method of claim 27, wherein the inserting the oral device includes grasping the handle between the index finger and the thumb and disposing the oral device in engagement with the tongue and the teeth of the subject without inserting the index finger or the thumb into the subject’s mouth.

37. The method of claim 27, wherein the inserting the oral device includes urging a first handle portion toward a second handle portion to transition the curved frame from a first configuration in which the first end and the second end of the curved frame are a first distance apart to a second configuration in which the first end and the second end of the curved frame are a second distance apart and inserting the oral device into the subject’s mouth.

38-41. (canceled)
